(19) United States
(12) Patent Application Publication
(10) Pub. No.: US 2022/0009878 A1
(43) Pub. Date: Jan. 13, 2022

(54) IONIZABLE AMINE LIPIDS

(71) Applicant: Intellia Therapeutics, Inc., Cambridge, MA (US)

(72) Inventors: Rubina Giare Parmar, Cambridge, MA (US); Stephen S. Scully, Cambridge, MA (US); Micah Maetani, Cambridge, MA (US); Derek LaPlaca, Cambridge, MA (US)

(21) Appl. No.: 17/282,157

(22) PCT Filed: Oct. 2, 2019
(86) PCT No.: PCT/US2019/054240

§ 371 Date: Apr. 1, 2021

Related U.S. Application Data
(60) Provisional application No. 62/740,274, filed on Oct. 2, 2018.

(51) Int. Cl. C07C 229/12; C07C 229/30; C07C 229/26; C07C 271/12; C07C 233/36; C07C 271/20; C07C 275/14; C07C 311/05; C12N 9/22; C12N 15/11; A61K 9/51

(52) U.S. Cl. CPC C07C 229/12; C07C 229/30; C07C 229/26; C07C 271/12; C07C 233/36; C12N 2310/20; C07C 275/14; C07C 311/05; C12N 9/22; C12N 15/111; A61K 9/5123; C07C 271/20

(57) ABSTRACT

The disclosure provides ionizable amine lipids and salts thereof (e.g., pharmaceutically acceptable salts thereof) useful for the delivery of biologically active agents, for example delivering biologically active agents to cells to prepare engineered cells. The ionizable amine lipids disclosed herein are useful as ionizable lipids in the formulation of lipid nanoparticle-based compositions.

### CROSS-REFERENCE TO RELATED APPLICATIONS

This application claims priority to U.S. Provisional Patent Application No. 62/740,274, filed Oct. 2, 2018, the entire contents of which are incorporated herein by reference.

### BACKGROUND

Lipid nanoparticles formulated with ionizable amine-containing lipids can serve as cargo vehicles for delivery of biologically active agents, in particular polynucleotides, such as RNAs, mRNAs, and guide RNAs into cells. The LNP compositions containing ionizable lipids can facilitate delivery of oligonucleotide agents across cell membranes, and can be used to introduce components and compositions for gene editing into living cells. Biologically active agents that are particularly difficult to deliver to cells include proteins, nucleic acid-based drugs, and derivatives thereof, particularly drugs that include relatively large oligonucleotides, such as mRNA. Compositions for delivery of promising gene editing technologies into cells, such as for delivery of CRISPR/Cas9 system components, are of particular interest (e.g., mRNA encoding a nuclease and associated guide RNA (gRNA)).

Compositions for delivery of the protein and nucleic acid components of CRISPR/Cas to a cell, such as a cell in a patient, are needed. In particular, compositions for delivering mRNA encoding the CRISPR protein component, and for delivering CRISPR guide RNAs are of particular interest. Compositions with useful properties for in vitro and in vivo delivery that can stabilize and deliver RNA components, are also of particular interest.

### BRIEF SUMMARY

The present disclosure provides amine-containing lipids useful for the formulation of lipid nanoparticle (LNP) compositions. Such LNP compositions may have properties advantageous for delivery of nucleic acid cargo, such as CRISPR/Cas gene editing components, to cells.

In certain embodiments, the invention relates to a compound of Formula I

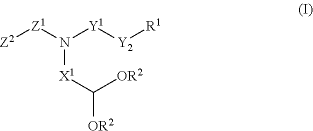

wherein, independently for each occurrence,
X¹ is C₅₋₁₁ alkylene,
Y¹ is C₃₋₁₁ alkylene,

### Y² is

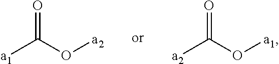

wherein a₁ is a bond to Y¹, and a₂ is a bond to R¹,
Z¹ is C₂₋₄ alkylene,
Z² is selected from —OH, —NH₂, —OC(═O)R³, —OC(═O)NHR³, —NHC(═O)NHR³, and —NHS(═O)₂R³,
R¹ is C₄₋₁₂ alkyl or C₃₋₁₂ alkenyl,
each R² is independently C₄₋₁₂ alkyl, and
R³ is C₁₋₃ alkyl,
or a salt thereof.

In certain embodiments, the invention relates to any compound described herein, wherein the salt is a pharmaceutically acceptable salt.

In certain embodiments, the invention relates to any compound described herein, wherein X¹ is linear C₅₋₁₁ alkylene, for example, linear C₆₋₁₀ alkylene, preferably linear C₇ alkylene or linear C₉ alkylene. In certain embodiments, X¹ is linear C₈ alkylene. In certain embodiments, X¹ is linear C₆ alkylene.

In certain embodiments, the invention relates to any compound described herein, wherein Y¹ is linear C₄₋₉ alkylene, for example, Y¹ is linear C₅₋₉ alkylene or linear C₆₋₈ alkylene, preferably Y¹ is linear C₇ alkylene.

In certain embodiments, the invention relates to any compound described herein, wherein Y² is

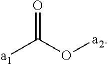

In certain embodiments, the invention relates to any compound described herein, wherein R¹ is C₄₋₁₂ alkenyl, such as C₉ alkenyl.

In certain embodiments, the invention relates to any compound described herein, wherein Y¹, Y², and R¹ are selected to form a linear chain of 16-21 atoms, preferably 16-18 atoms.

In certain embodiments, the invention relates to any compound described herein, wherein Z¹ is linear C₂₋₄ alkylene, preferably Z¹ is C₂ alkylene or C₃ alkylene.

In certain embodiment, Z² is —OH. In some embodiments, Z² is —NH₂. In certain embodiments, Z² is selected from —OC(═O)R³, —OC(═O)NHR³, —NHC(═O)NHR³, and —NHS(═O)₂R³, for example, Z² is —OC(═O)R³ or —OC(═O)NHR³. In some embodiments, Z² is —NHC(═O)NHR³ or —NHS(═O)₂R³.

In certain embodiments, R³ is methyl.

In certain embodiments, the invention relates to any compound described herein, wherein R¹ is linear C₄₋₁₂ alkyl, for example, R¹ is linear C₆₋₁₁ alkyl, such as linear C₈₋₁₀ alkyl, preferably R¹ is linear C₉ alkyl.

In certain embodiments, the invention relates to any compound described herein, wherein R¹ is branched C₆₋₁₂ alkyl, for example, R¹ is branched C₇₋₁₁ alkyl, such as branched C₈ alkyl, branched C₉ alkyl, or branched C₁₀ alkyl.

In certain embodiments, the invention relates to any compound described herein, wherein each R², independently, is C₅₋₁₂ alkyl, such as linear C₅₋₁₂ alkyl. In some embodiments the invention relates to any compound described herein, wherein each R², independently, is linear C₆₋₁₀ alkyl, for example linear C₆₋₈ alkyl.

In certain embodiments, the invention relates to any compound described herein, wherein each R², independently, is branched C₅₋₁₂ alkyl. In some embodiments the invention relates to any compound described herein, wherein each R², independently, is branched C₆₋₁₀ alkyl, for example branched C₇₋₉ alkyl, such as branched C₈ alkyl.

In certain embodiments, the invention relates to any compound described herein, wherein X¹ and one of the R² moieties are selected to form a linear chain of 16-18 atoms, including the carbon and oxygen atoms of the acetal.

In certain embodiments, the invention relates to a compound of Formula II

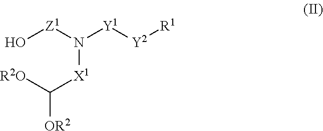

wherein, independently for each occurrence,
X¹ is C₅₋₁₁ alkylene,
Y¹ is C₃₋₁₀ alkylene,

### Y² is

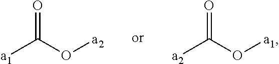

wherein a₁ is a bond to Y¹, and a₂ is a bond to R¹,
Z¹ is C₂₋₄ alkylene, R⁶¹ is C₄₋₁₂ alkyl or C₃₋₁₂ alkenyl,
each R² is independently C₄₋₁₂ alkyl,
or a salt thereof.

In certain embodiments, the invention relates to any compound described herein, wherein the salt is a pharmaceutically acceptable salt.

In certain embodiments, the invention relates to any compound described herein, wherein X¹ is linear C₅₋₁₁ alkylene, for example, linear C₆₋₈ alkylene, preferably linear C₇ alkylene.

In certain embodiments, the invention relates to any compound described herein, wherein Y¹ is linear C₅₋₉ alkylene, for example, Y¹ is C₄₋₉ alkylene or linear C₆₋₈ alkylene, preferably Y¹ is linear C₇ alkylene.

In certain embodiments, the invention relates to any compound described herein, wherein Y² is

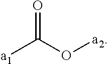

In certain embodiments, the invention relates to any compound described herein, wherein R¹ is C₄₋₁₂ alkenyl.

In certain embodiments, the invention relates to any compound described herein, wherein Y¹, Y², and R¹ are selected to form a linear chain of 16-21 atoms, preferably 16-18 atoms.

In certain embodiments, the invention relates to any compound described herein, wherein Z¹ is linear C₂₋₄ alkylene, preferably Z¹ is C₂ alkylene.

In certain embodiments, the invention relates to any compound described herein, wherein R¹ is linear C₄₋₁₂ alkyl, for example, R¹ is linear C₈₋₁₀ alkyl, preferably R¹ is linear C₉ alkyl.

In certain embodiments, the invention relates to any compound described herein, wherein each R² is C₅₋₁₂ alkyl such as linear C₅₋₁₂ alkyl. In some embodiments the invention relates to any compound described herein, wherein each R² is linear C₆₋₁₀ alkyl, for example linear C₆₋₈ alkyl.

In certain embodiments, the invention relates to any compound described herein, wherein X¹ and one of the R² moieties are selected to form a linear chain of 16-18 atoms, including the carbon and oxygen atoms of the acetal.

In certain embodiments, the invention relates to a compound selected from:

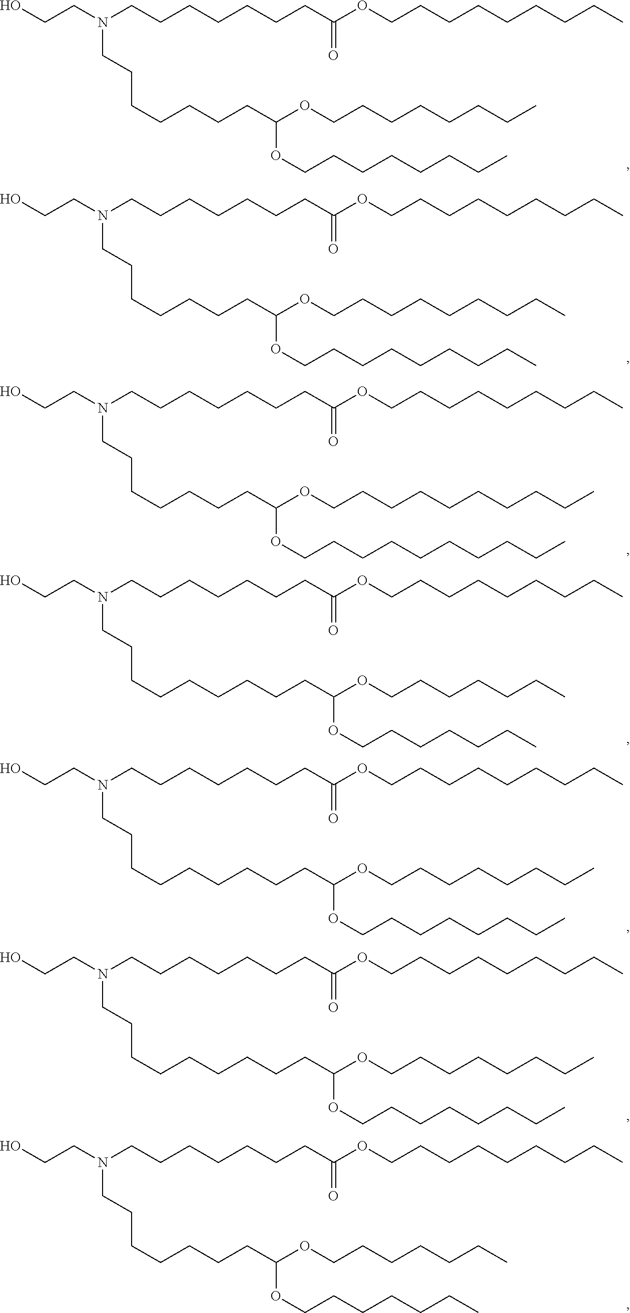

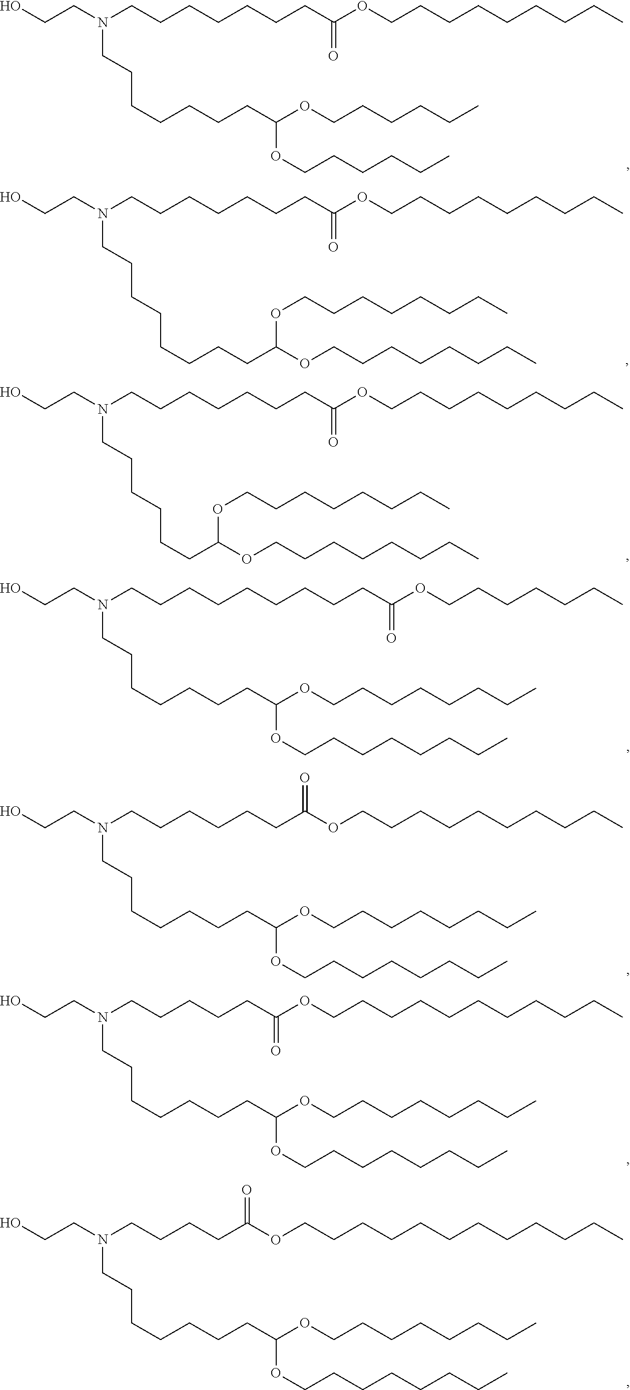

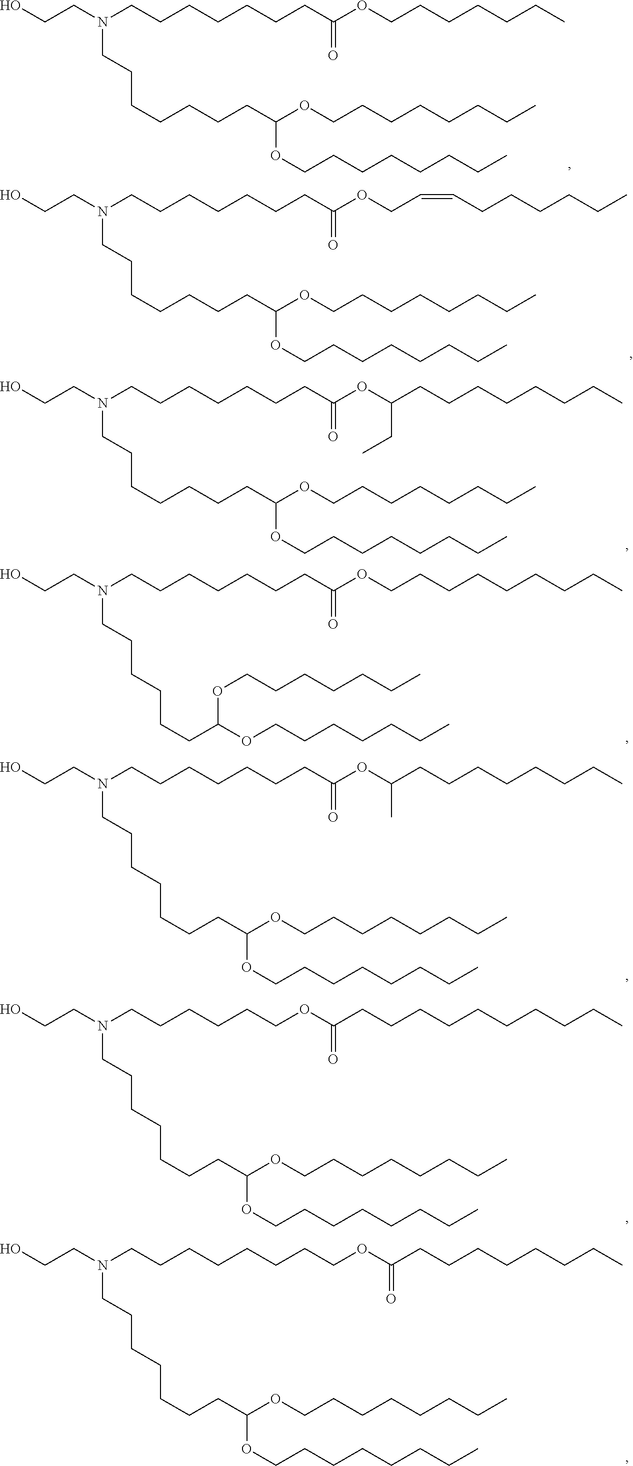

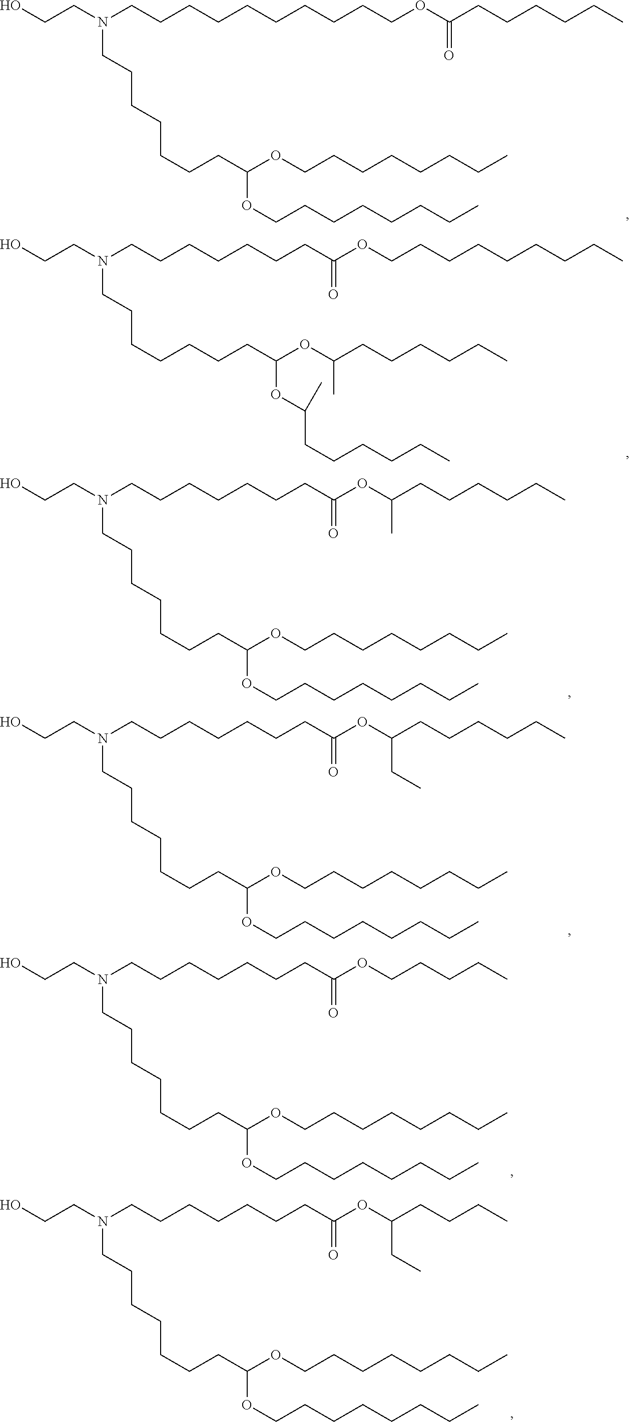

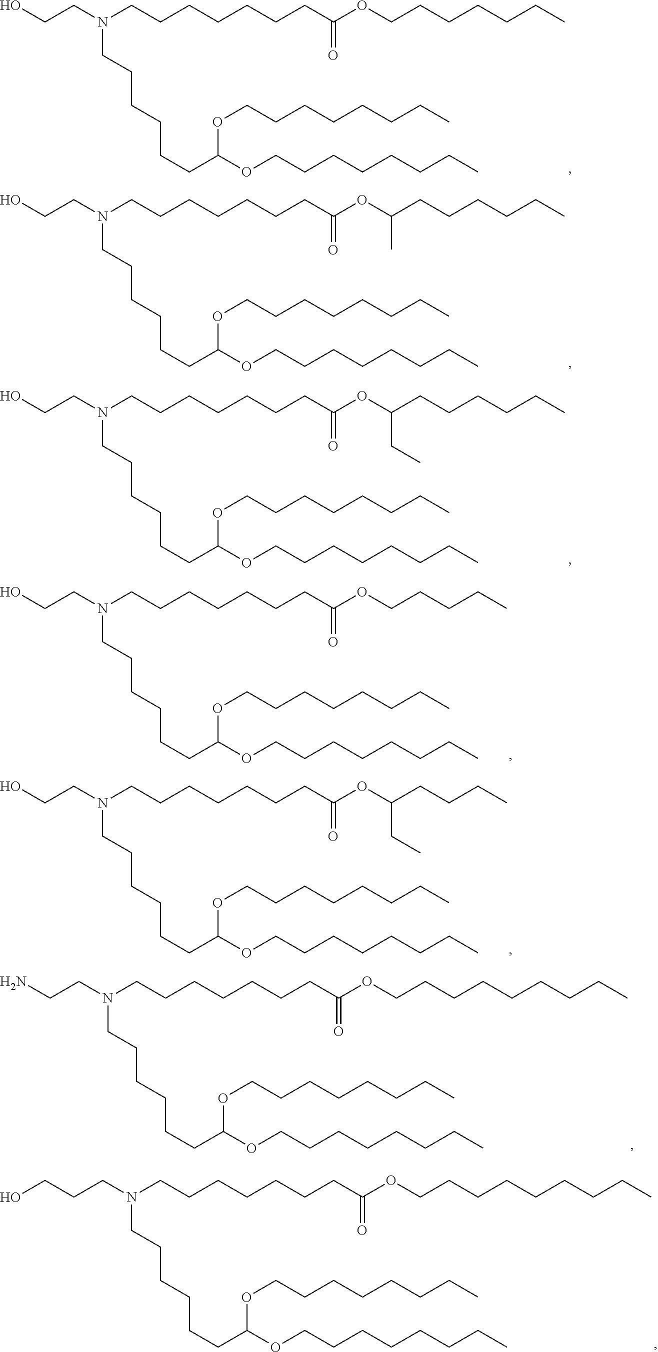

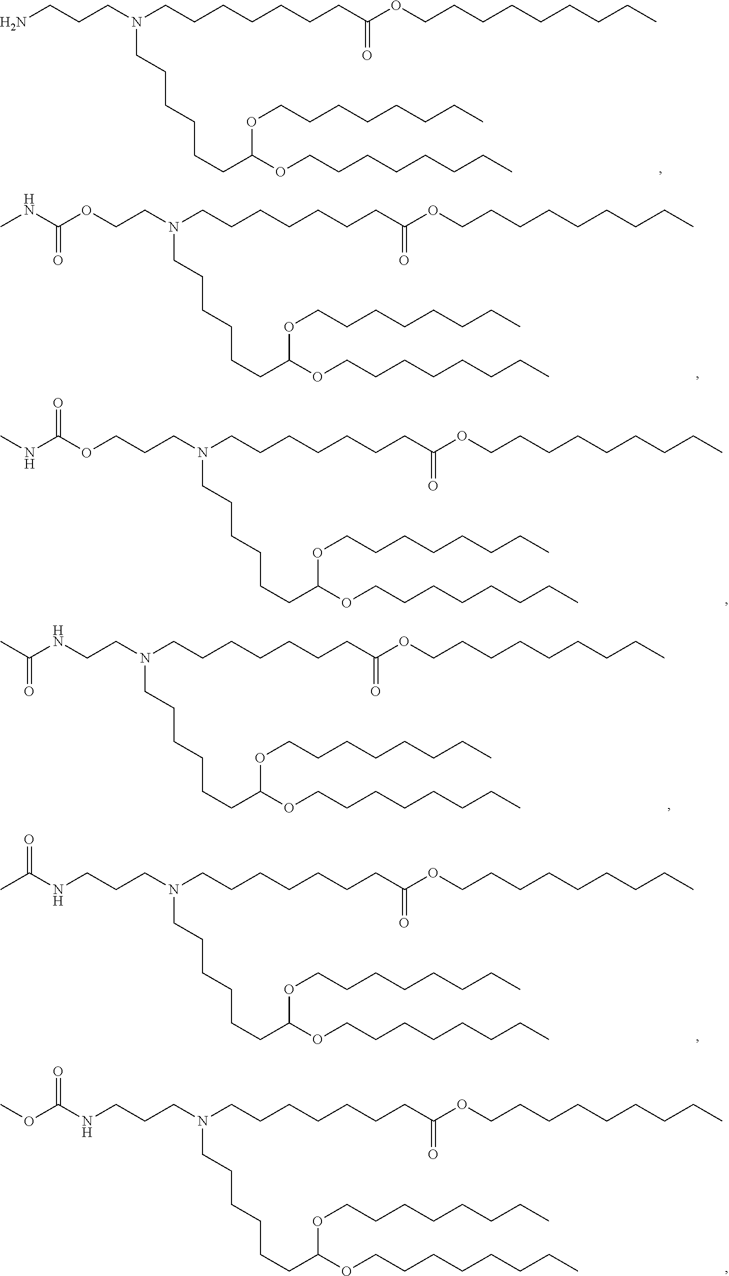

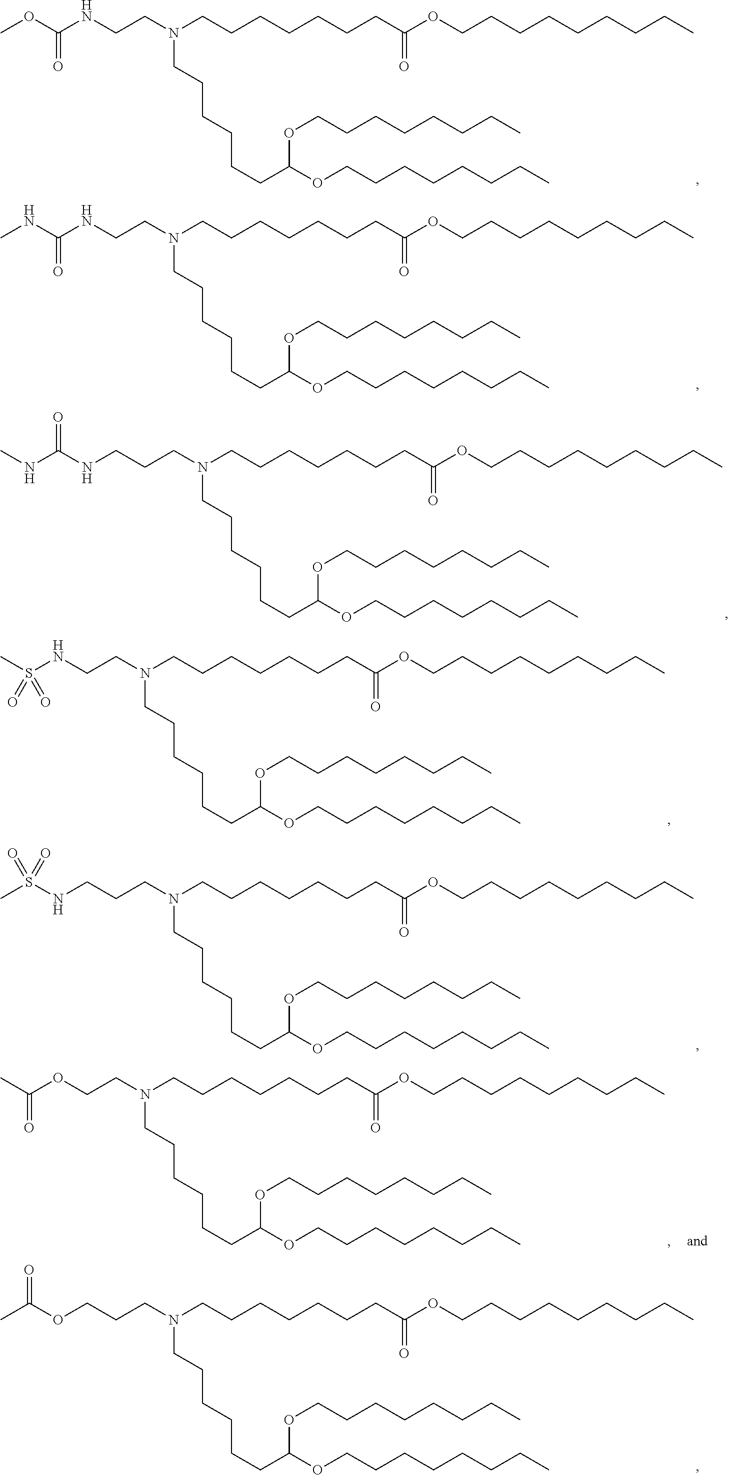

or a salt thereof, preferably a pharmaceutically acceptable salt.

In certain embodiments, the invention relates to any compound described herein, wherein the pKa of the protonated form of the compound is from about 5.1 to about 8.0, for example from about 5.7 to about 6.5, from about 5.7 to about 6.4, or from about 5.8 to about 6.2. In some embodiments, the pKa of the protonated form of the compound is from about 5.5 to about 6.0. In certain embodiments, the pKa of the protonated form of the compound is from about 6.1 to about 6.3.

In certain embodiments, the invention relates to a composition comprising any compound described herein and a lipid component, for example comprising about 50% of a compound of any one of the preceding claims and a lipid component, for example, an amine lipid, preferably a compound of Formula(I) or Formula (II).

In certain embodiments, the invention relates to any composition described herein, wherein the composition is an LNP composition. For example, the invention relates to an LNP composition comprising any compound described herein and a lipid component. In certain embodiments, the invention relates to any LNP composition described herein, wherein the lipid component comprises a helper lipid and a PEG lipid. In certain embodiments, the invention relates to any LNP composition described herein, wherein the lipid component comprises a helper lipid, a PEG lipid, and a neutral lipid. In certain embodiments, the invention relates to any LNP composition described herein, further comprising a cryoprotectant. In certain embodiments, the invention relates to any LNP composition described herein, further comprising a buffer.

In certain embodiments, the invention relates to any LNP composition described herein, further comprising a nucleic acid component. In certain embodiments, the invention relates to any LNP composition described herein, further comprising an RNA or DNA component. In certain embodiments, the invention relates to any LNP composition described herein, wherein the LNP composition has an N/P ratio of about 3-10, for example the N/P ratio is about 6±1, or the N/P ratio is about 6±0.5. In certain embodiments, the invention relates to any LNP composition described herein, wherein the LNP composition has an N/P ratio of about 6.

In certain embodiments, the invention relates to any LNP composition described herein, wherein the RNA component comprises an mRNA. In certain embodiments, the invention relates to any LNP composition described herein, wherein the RNA component comprises an RNA-guided DNA-binding agent, for example a Cas nuclease mRNA, such as a Class 2 Cas nuclease mRNA, or a Cas9 nuclease mRNA.

In certain embodiments, the invention relates to any LNP composition described herein, wherein the mRNA is a modified mRNA. In certain embodiments, the invention relates to any LNP composition described herein, wherein the RNA component comprises a gRNA nucleic acid. In certain embodiments, the invention relates to any LNP composition described herein, wherein the gRNA nucleic acid is a gRNA.

In certain embodiments, the invention relates to an LNP composition described herein, wherein the RNA component comprises a Class 2 Cas nuclease mRNA and a gRNA. In certain embodiments, the invention relates to any LNP composition described herein, wherein the gRNA nucleic acid is or encodes a dual-guide RNA (dgRNA). In certain embodiments, the invention relates to any LNP composition described herein, wherein the gRNA nucleic acid is or encodes a single-guide RNA (sgRNA).

In certain embodiments, the invention relates to any LNP composition described herein, wherein the gRNA is a modified gRNA. In certain embodiments, the invention relates to any LNP composition described herein, wherein the modified gRNA comprises a modification at one or more of the first five nucleotides at a 5′ end. In certain embodiments, the invention relates to any LNP composition described herein, wherein the modified gRNA comprises a modification at one or more of the last five nucleotides at a 3′ end.

In certain embodiments, the invention relates to any LNP composition described herein, further comprising at least one template nucleic acid.

In certain embodiments, the invention relates to a method of gene editing, comprising contacting a cell with an LNP. In certain embodiments, the invention relates to any method of gene editing described herein, comprising cleaving DNA.

In certain embodiments, the invention relates to a method of cleaving DNA, comprising contacting a cell with an LNP composition. In certain embodiments, the invention relates to any method of cleaving DNA described herein, wherein the cleaving step comprises introducing a single stranded DNA nick. In certain embodiments, the invention relates to any method of cleaving DNA described herein, wherein the cleaving step comprises introducing a double-stranded DNA break. In certain embodiments, the invention relates to any method of cleaving DNA described herein, wherein the LNP composition comprises a Class 2 Cas mRNA and a guide RNA nucleic acid. In certain embodiments, the invention relates to any method of cleaving DNA described herein, further comprising introducing at least one template nucleic acid into the cell. In certain embodiments, the invention relates to any method of cleaving DNA described herein, comprising contacting the cell with an LNP composition comprising a template nucleic acid.

In certain embodiments, the invention relates to any a method of gene editing described herein, wherein the method comprises administering the LNP composition to an animal, for example a human. In certain embodiments, the invention relates to any method of gene editing described herein, wherein the method comprises administering the LNP composition to a cell, such as a eukaryotic cell.

In certain embodiments, the invention relates to any method of gene editing described herein, wherein the method comprises administering the mRNA formulated in a first LNP composition and a second LNP composition comprising one or more of an mRNA, a gRNA, a gRNA nucleic acid, and a template nucleic acid. In certain embodiments, the invention relates to any method of gene editing described herein, wherein the first and second LNP compositions are administered simultaneously. In certain embodiments, the invention relates to any method of gene editing described herein, wherein the first and second LNP compositions are administered sequentially. In certain embodiments, the invention relates to any method of gene editing described herein, wherein the method comprises administering the mRNA and the guide RNA nucleic acid formulated in a single LNP composition.

In certain embodiments, the invention relates to any method of gene editing described herein, wherein the gene editing results in a gene knockout.

In certain embodiments, the invention relates to any method of gene editing described herein, wherein the gene editing results in a gene correction.

### BRIEF DESCRIPTION OF DRAWINGS

FIG. 1 is a graph showing percentage of editing of B2M in mouse liver cells after delivery using LNPs comprising a compound of Formula(I) or Formula (II) or a control, as described in Example 52.

FIG. 2A is a graph showing percentage of editing of TTR in mouse liver cells after delivery using LNPs comprising Compound 19, a compound of Formula(I) or Formula (II) (Compound 1), or a control, as described in Example 53. Dose response data are also shown.

FIG. 2B is a graph showing serum TTR (μg/mL), as described in Example 53. Dose response data are also shown.

FIG. 2C is a graph showing serum TTR (% TSS), as described in Example 53. Dose response data are also shown.

FIG. 3 is a graph showing dose response percentage of editing of B2M in mouse liver cells after delivery using LNPs comprising Compound 19, a compound of Formula(I) or Formula (II) (Compound 1), or a control, as described in Example 53.

FIG. 4 is a graph showing dose response percentage of editing of B2M in mouse liver cells after delivery using LNPs comprising Compound 19, a compound of Formula(I) or Formula (II) (Compound 4), or a control, as described in Example 54.

FIG. 5A is a graph showing percentage of editing of TTR in mouse liver cells after delivery using LNPs comprising Compound 19, a compound of Formula(I) or Formula (II), or a control, as described in Example 55. Dose response data are also shown.

FIG. 5B is a graph showing serum TTR (μg/mL), as described in Example 55. Dose response data are also shown.

FIG. 5C is a graph showing serum TTR (% TSS), as described in Example 55. Dose response data are also shown.

FIG. 6A is a graph showing percentage of editing of TTR in mouse liver cells after delivery using LNPs comprising Compound 19, a compound of Formula(I) or Formula (II), or a control, as described in Example 58.

FIG. 6B is a graph showing serum TTR (μg/mL), as described in Example 58.

FIG. 7A is a graph showing percentage of editing of TTR in mouse liver cells after delivery using LNPs comprising Compound 19, a compound of Formula(I) or Formula (II), or a control, as described in Example 59.

FIG. 7B is a graph showing serum TTR (μg/mL), as described in Example 59.

FIG. 8A is a graph showing percentage of editing of TTR in mouse liver cells after delivery using LNPs comprising Compound 19, a compound of Formula(I) or Formula (II), or a control, as described in Example 60.

FIG. 8B is a graph showing serum TTR (μg/mL), as described in Example 60.

FIG. 9A is a graph showing percentage of editing of TTR in mouse liver cells after delivery using LNPs comprising Compound 19, a compound of Formula(I) or Formula (II), or a control, as described in Example 61.

FIG. 9B is a graph showing serum TTR (μg/mL), as described in Example 61.

FIG. 10A is a graph showing percentage of editing of TTR in mouse liver cells after delivery using LNPs comprising Compound 19, a compound of Formula(I) or Formula (II), or a control, as described in Example 62.

FIG. 10B is a graph showing serum TTR (μg/mL), as described in Example 62.

### DETAILED DESCRIPTION

The present disclosure provides lipids, particularly ionizable lipids, useful for delivering biologically active agents, including nucleic acids, such as CRISPR/Cas component RNAs (the “cargo”), to a cell, and methods for preparing and using such compositions. The lipids and pharmaceutically acceptable salts thereof are provided, optionally as compositions comprising the lipids, including LNP compositions. In certain embodiments, the LNP composition may comprise a biologically active agent, e.g. an RNA component, and a lipid component that includes a compound of Formula(I) or Formula (II), as defined herein. In certain embodiments, the RNA component includes an RNA. In some embodiments, the RNA component comprises a nucleic acid. In some embodiments, the lipids are used to deliver a biologically active agent, e.g. a nucleic acid such as an mRNA to a cell such as a liver cell. In certain embodiments, the RNA component includes a gRNA and optionally an mRNA encoding a Class 2 Cas nuclease. Methods of gene editing and methods of making engineered cells using these compositions are also provided.

### Lipid Nanoparticle Compositions

Disclosed herein are various LNP compositions for delivering biologically active agents, such as nucleic acids, e.g., mRNAs and guide RNAs, including CRISPR/Cas cargoes. Such LNP compositions include an “ionizable amine lipid”, along with a neutral lipid, a PEG lipid, and a helper lipid. “Lipid nanoparticle” or “LNP” refers to, without limiting the meaning, a particle that comprises a plurality of (i.e., more than one) LNP components physically associated with each other by intermolecular forces.

### Lipids

The disclosure provides lipids that can be used in LNP compositions.

In certain embodiments, the invention relates to a compound of Formula I

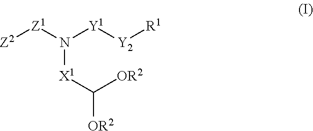

wherein, independently for each occurrence,
X¹ is C₅₋₁₁ alkylene,
Y¹ is C₃₋₁₁ alkylene,

### Y² is

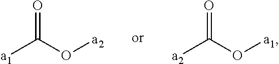

wherein a₁ is a bond to Y¹, and a₂ is a bond to R¹,
Z¹ is C₂₋₄ alkylene,
Z² is selected from —OH, —NH₂, —OC(═O)R³, —OC(═O)NHR³, —NHC(═O)NHR³, and —NHS(═O)₂R³,
R¹ is C₄₋₁₂ alkyl or C₃₋₁₂ alkenyl,
each R² is independently C₄₋₁₂ alkyl, and
R³ is C₁₋₃ alkyl,
or a salt thereof.

In certain embodiments, the invention relates to any compound described herein, wherein the salt is a pharmaceutically acceptable salt.

In certain embodiments, the invention relates to any compound described herein, wherein X¹ is linear C₅₋₁₁ alkylene, for example, linear C₆₋₁₀ alkylene, preferably linear C₇ alkylene or linear C₉ alkylene. In certain embodiments, X¹ is linear C₈ alkylene. In certain embodiments, X¹ is linear C₆ alkylene.

In certain embodiments, the invention relates to any compound described herein, wherein Y¹ is linear C₄₋₉ alkylene, for example, Y¹ is linear C₅₋₉ alkylene or linear C₆₋₈ alkylene, preferably Y¹ is linear C₇ alkylene.

In certain embodiments, the invention relates to any compound described herein, wherein Y² is

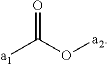

In certain embodiments, the invention relates to any compound described herein, wherein R¹ is C₄₋₁₂ alkenyl, such as C₉ alkenyl.

In certain embodiments, the invention relates to any compound described herein, wherein Y¹, Y², and R¹ are selected to form a linear chain of 16-21 atoms, preferably 16-18 atoms.

In certain embodiments, the invention relates to any compound described herein, wherein Z¹ is linear C₂₋₄ alkylene, preferably Z¹ is C₂ alkylene or C₃ alkylene.

In certain embodiment, Z² is —OH. In some embodiments, Z² is —NH₂. In certain embodiments, Z² is selected from —OC(═O)R³, —OC(═O)NHR³, —NHC(═O)NHR³, and —NHS(═O)₂R³, for example, Z² is —OC(═O)R³ or —OC(═O)NHR³. In some embodiments, Z² is —NHC(═O)NHR³ or —NHS(═O)₂R³.

In certain embodiments, R³ is methyl.

In certain embodiments, the invention relates to any compound described herein, wherein R¹ is linear C₄₋₁₂ alkyl, for example, R¹ is linear C₆₋₁₁ alkyl, such as linear C₈₋₁₀ alkyl, preferably R¹ is linear C₉ alkyl.

In certain embodiments, the invention relates to any compound described herein, wherein R¹ is branched C₆₋₁₂ alkyl, for example, R¹ is branched C₇₋₁₁ alkyl, such as branched C₈ alkyl, branched C₉ alkyl, or branched C₁₀ alkyl.

In certain embodiments, the invention relates to any compound described herein, wherein each R², independently, is C₅₋₁₂ alkyl, such as linear C₅₋₁₂ alkyl. In some embodiments the invention relates to any compound described herein, wherein each R², independently, is linear C₆₋₁₀ alkyl, for example linear C₆₋₈ alkyl.

In certain embodiments, the invention relates to any compound described herein, wherein each R², independently, is branched C₅₋₁₂ alkyl. In some embodiments the invention relates to any compound described herein, wherein each R², independently, is branched C₆₋₁₀ alkyl, for example branched C₇₋₉ alkyl, such as branched C₈ alkyl.

In certain embodiments, the invention relates to any compound described herein, wherein X¹ and one of the R² moieties are selected to form a linear chain of 16-18 atoms, including the carbon and oxygen atoms of the acetal.

In certain embodiments, the lipid is a compound having a structure of Formula (II):

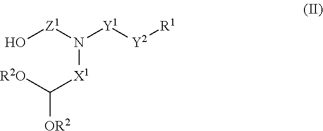

wherein, independently for each occurrence,
X¹ is C₅₋₁₁ alkylene;
Y¹ is C₃₋₁₀ alkylene;
Y² is

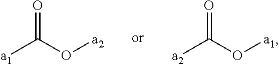

wherein a₁ is a bond to Y¹, and a₂ is a bond to R¹;
Z¹ is C₂₋₄ alkylene;
R¹ is C₄₋₁₂ alkyl or C₃₋₁₂ alkenyl; and
each R² is independently C₄₋₁₂ alkyl,
   or a salt thereof, such as a pharmaceutically acceptable salt thereof.

In some embodiments X¹ is linear C₅₋₁₁ alkylene, preferably a linear C₆₋₈ alkylene, more preferably a C₇ alkylene.

In certain embodiments, Y¹ is a linear C₅₋₉ alkylene, for example a linear C₆₋₈ alkylene or a linear C₄₋₉ alkylene, preferably a linear C₇ alkylene.

In certain embodiments Y² is

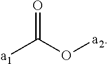

In some embodiments R¹ is C₄₋₁₂ alkyl, preferably a linear C₈₋₁₀ alkyl, more preferably a linear C₉ alkyl. In some embodiments R¹ is C₄₋₁₂ alkenyl.

In certain embodiments Z¹ is a linear C₂₋₄ alkylene, preferably a C₂ alkylene.

In certain embodiments R² is linear C₅₋₁₂ alkyl, for example a linear C₆₋₁₀ alkyl, such as a liner C₆₋₈ alkyl.

Representative compounds of Formula (I) include:

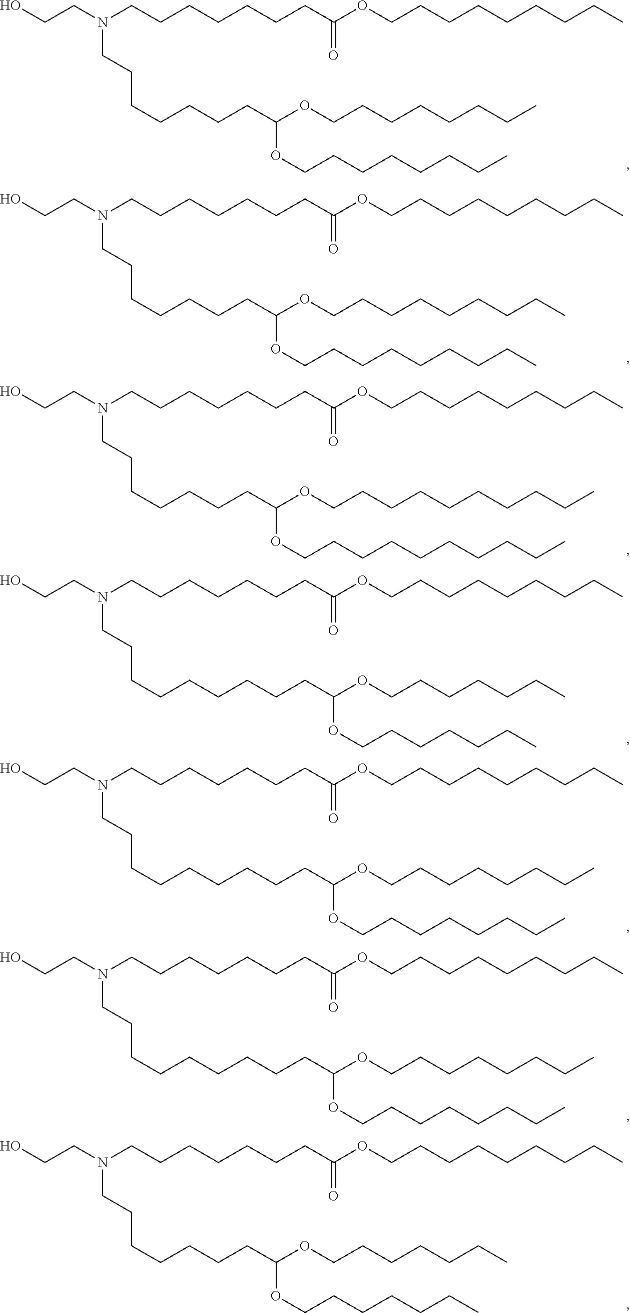

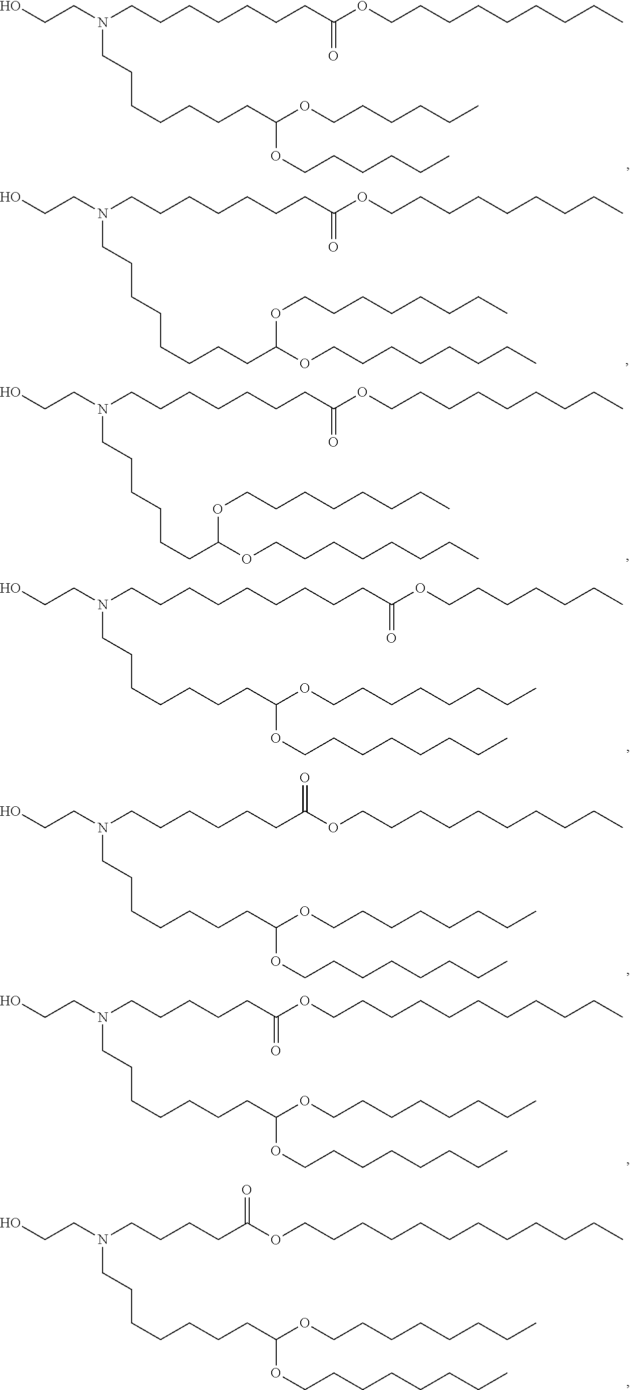

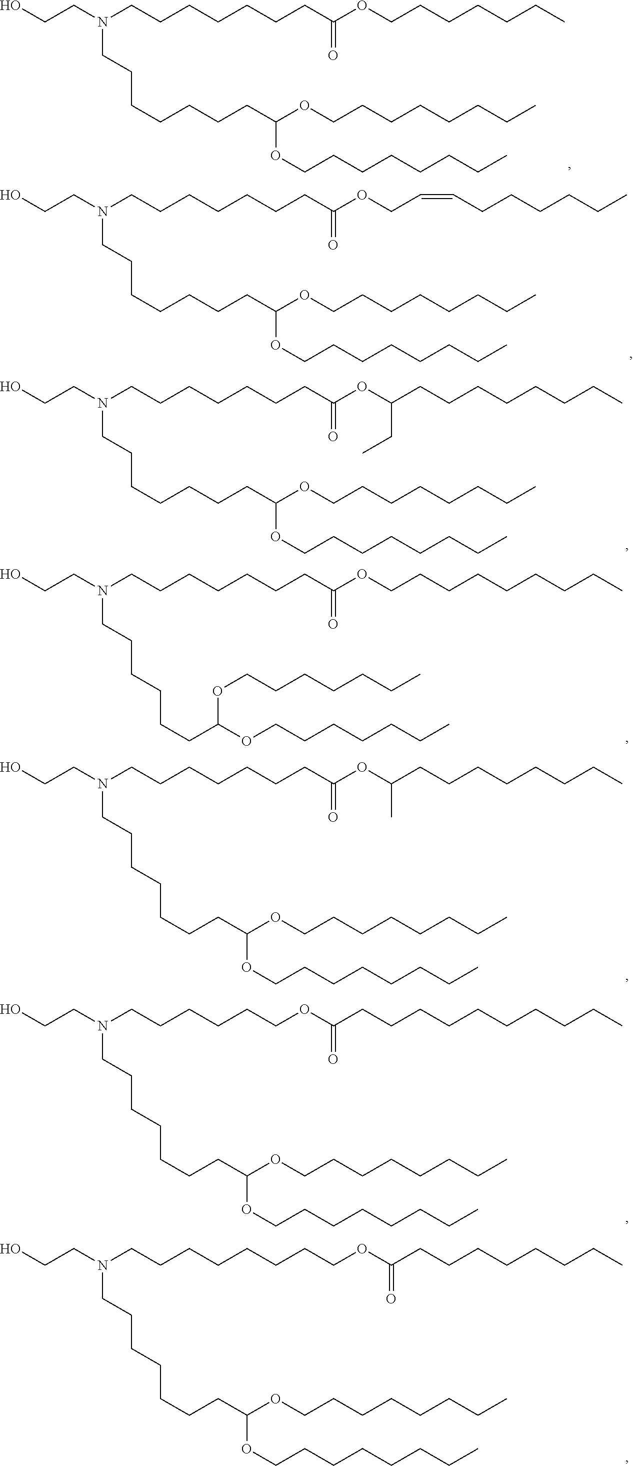

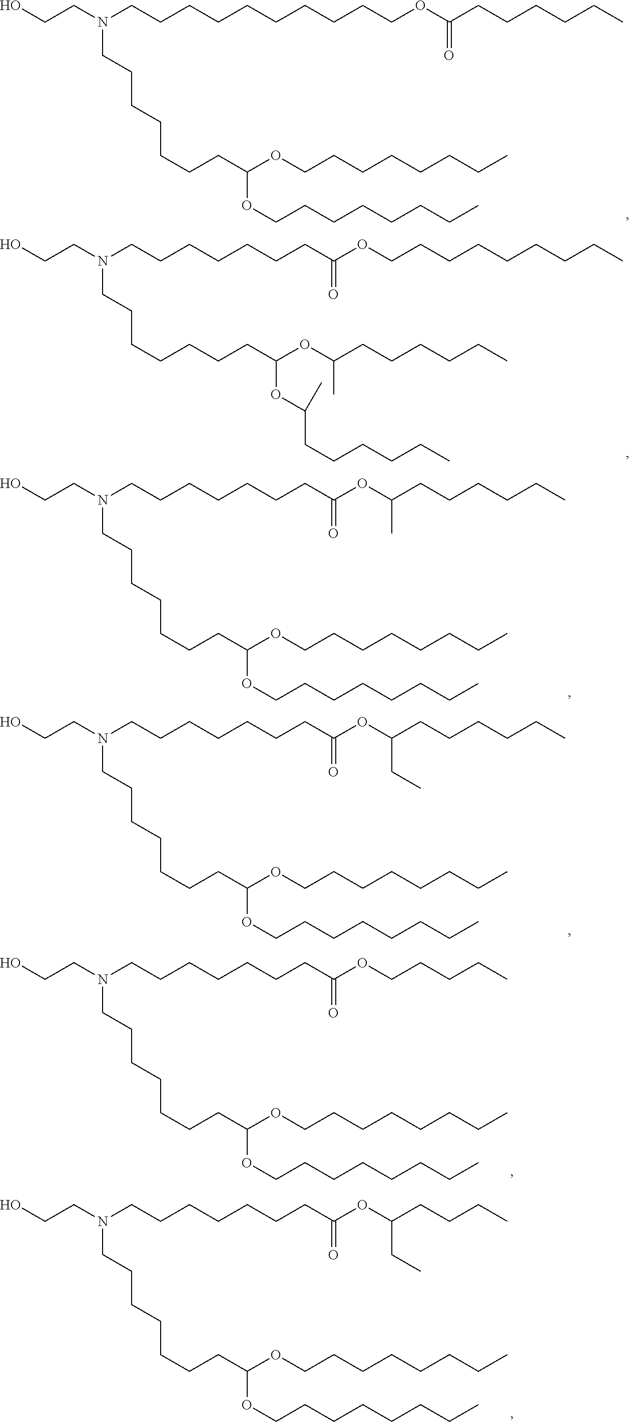

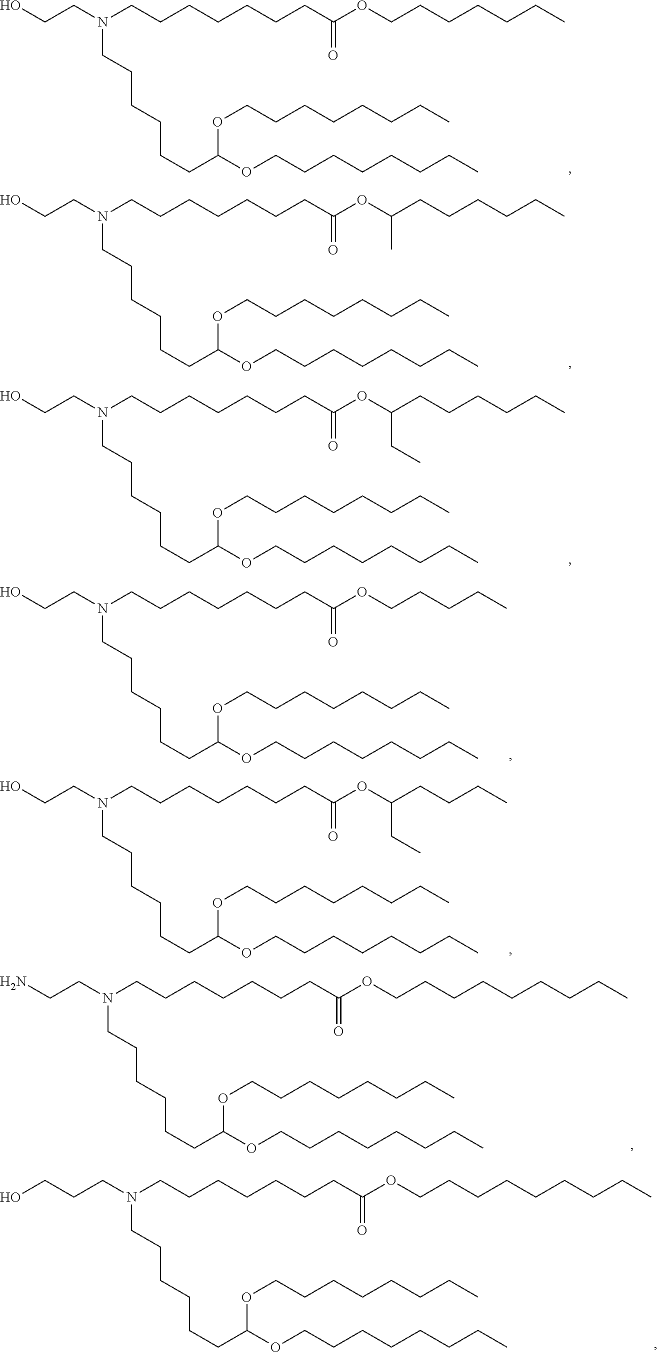

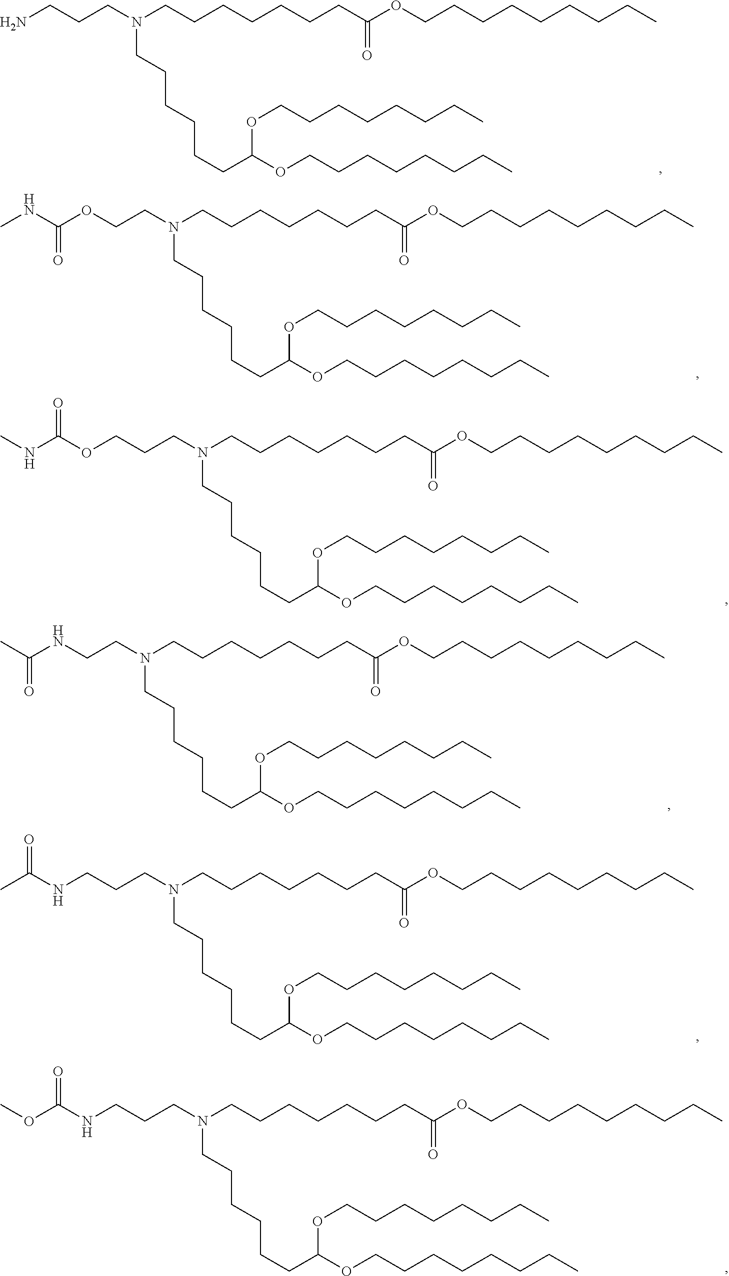

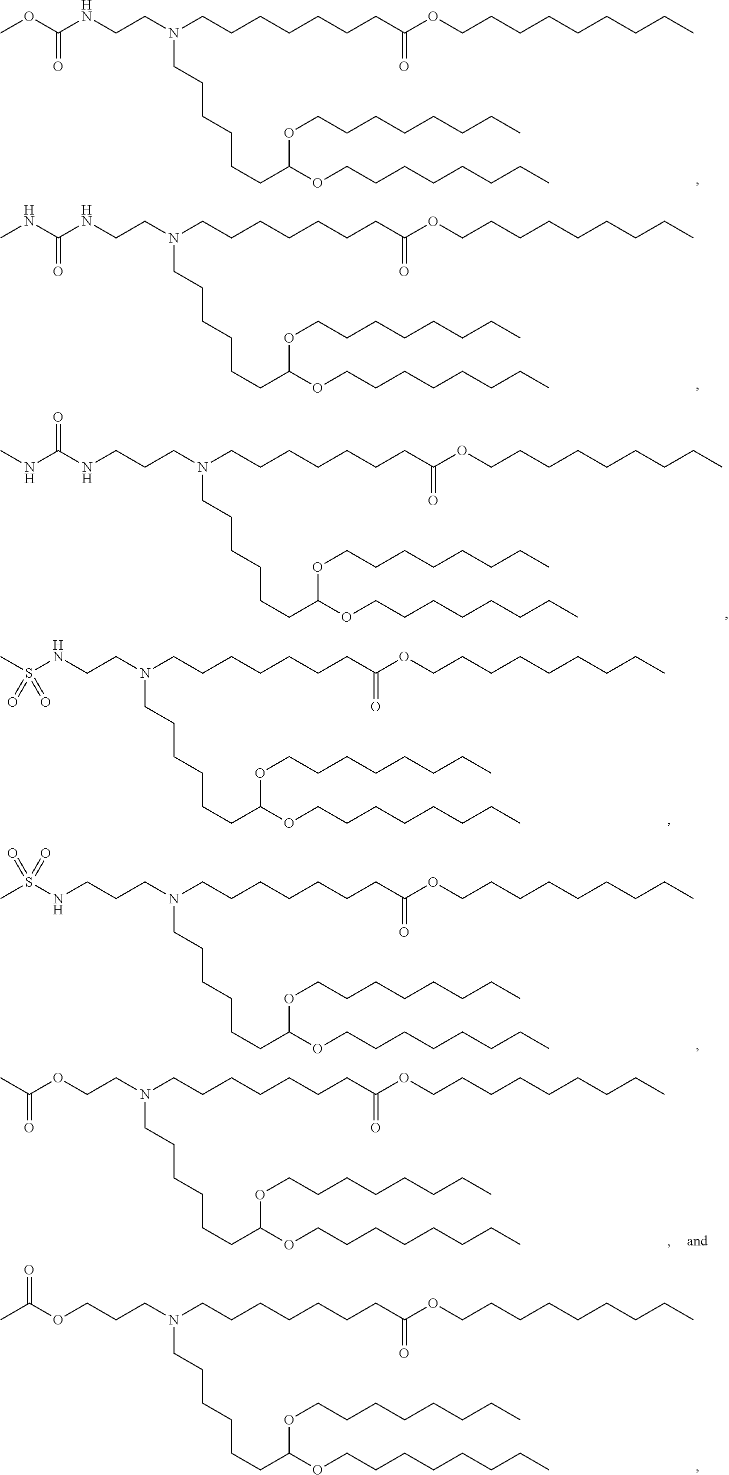

In certain embodiments, at least 75% of the compound of Formula(I) or Formula (II) of lipid compositions formulated as disclosed herein is cleared from the subject's plasma within 8, 10, 12, 24, or 48 hours, or 3, 4, 5, 6, 7, or 10 days after administration. In certain embodiments, at least 50% of the lipid compositions comprising a compound of Formula(I) or Formula (II) as disclosed herein are cleared from the subject's plasma within 8, 10, 12, 24, or 48 hours, or 3, 4, 5, 6, 7, or 10 days after administration, which can be determined, for example, by measuring a lipid (e.g. a compound of Formula(I) or Formula (II)), RNA (e.g. mRNA), or other component in the plasma. In certain embodiments, lipid-encapsulated versus free lipid, RNA, or nucleic acid component of the lipid composition is measured.

Lipid clearance may be measured as described in literature. See Maier, M. A., et al. Biodegradable Lipids Enabling Rapidly Eliminated Lipid Nanoparticles for Systemic Delivery of RNAi Therapeutics. Mol. Ther. 2013, 21(8), 1570-78 (“Maier”). For example, in Maier, LNP-siRNA systems containing luciferases-targeting siRNA were administered to six- to eight-week old male C57Bl/6 mice at 0.3 mg/kg by intravenous bolus injection via the lateral tail vein. Blood, liver, and spleen samples were collected at 0.083, 0.25, 0.5, 1, 2, 4, 8, 24, 48, 96, and 168 hours post-dose. Mice were perfused with saline before tissue collection and blood samples were processed to obtain plasma. All samples were processed and analyzed by LC-MS. Further, Maier describes a procedure for assessing toxicity after administration of LNP-siRNA compositions. For example, a luciferase-targeting siRNA was administered at 0, 1, 3, 5, and 10 mg/kg (5 animals/group) via single intravenous bolus injection at a dose volume of 5 mL/kg to male Sprague-Dawley rats. After 24 hours, about 1 mL of blood was obtained from the jugular vein of conscious animals and the serum was isolated. At 72 hours post-dose, all animals were euthanized for necropsy. Assessment of clinical signs, body weight, serum chemistry, organ weights and histopathology was performed. Although Maier describes methods for assessing siRNA-LNP compositions, these methods may be applied to assess clearance, pharmacokinetics, and toxicity of administration of lipid compositions, such as LNP compositions, of the present disclosure.

In certain embodiments, lipid compositions using the compounds of Formula(I) or Formula (II) disclosed herein exhibit an increased clearance rate relative to alternative ionizable amine lipids. In some such embodiments, the clearance rate is a lipid clearance rate, for example the rate at which a compound of Formula(I) or Formula (II) is cleared from the blood, serum, or plasma. In some embodiments, the clearance rate is a cargo (e.g. biologically active agent) clearance rate, for example the rate at which a cargo component is cleared from the blood, serum, or plasma. In some embodiments, the clearance rate is an RNA clearance rate, for example the rate at which an mRNA or a gRNA is cleared from the blood, serum, or plasma. In some embodiments, the clearance rate is the rate at which LNP is cleared from the blood, serum, or plasma. In some embodiments, the clearance rate is the rate at which LNP is cleared from a tissue, such as liver tissue or spleen tissue. Desirably, a high rate of clearance can result in a safety profile with no substantial adverse effects, and/or reduced LNP accumulation in circulation and/or in tissues.

The compounds of Formula(I) or Formula (II) of the present disclosure may form salts depending upon the pH of the medium they are in. For example, in a slightly acidic medium, the compounds of Formula(I) or Formula (II) may be protonated and thus bear a positive charge. Conversely, in a slightly basic medium, such as, for example, blood where pH is approximately 7.35, the compounds of Formula(I) or Formula (II) may not be protonated and thus bear no charge. In some embodiments, the compounds of Formula(I) or Formula (II) of the present disclosure may be predominantly protonated at a pH of at least about 9. In some embodiments, the compounds of Formula(I) or Formula (II) of the present disclosure may be predominantly protonated at a pH of at least about 10.

The pH at which a compound of Formula (I) or Formula (II) is predominantly protonated is related to its intrinsic pKa. In preferred embodiments, a salt of a compound of Formula (I) or Formula (II) of the present disclosure has a pKa in the range of from about 5.1 to about 8.0, even more preferably from about 5.5 to about 7.5, for example from about 6.1 to about 6.3. In preferred other embodiments, a salt of a compound of Formula (I) of the present disclosure has a pKa in the range of from about 5.3 to about 8.0, e.g., from about 5.7 to about 6.5. In other embodiments, a salt of a compound of Formula(I) or Formula (II) of the present disclosure has a pKa in the range of from about 5.7 to about 6.4, e.g., from about 5.8 to about 6.2. In other preferred embodiments, a salt of a compound of Formula (I) of the present disclosure has a pKa in the range of from about 5.7 to about 6.5, e.g., from about 5.8 to about 6.4. Alternatively, a salt of a compound of Formula(I) or Formula (II) of the present disclosure has a pKa in the range of from about 5.8 to about 6.5. In some embodiments, the pKa of the protonated form of the compound of Formula(I) or Formula (II) is from about 5.5 to about 6.0. A salt of a compound of Formula(I) or Formula (II) of the present disclosure may have a pKa in the range of from about 6.0 to about 8.0, preferably from about 6.0 to about 7.5. The pKa of a salt of a compound of Formula(I) or Formula (II) can be an important consideration in formulating LNPs, as it has been found that LNPs formulated with certain lipids having a pKa ranging from about 5.5 to about 7.0 are effective for delivery of cargo in vivo, e.g. to the liver. Further, it has been found that LNPs formulated with certain lipids having a pKa ranging from about 5.3 to about 6.4 are effective for delivery in vivo, e.g. to tumors. See, e.g., WO 2014/136086.

### Additional Lipids

“Neutral lipids” suitable for use in a lipid composition of the disclosure include, for example, a variety of neutral, uncharged or zwitterionic lipids. Examples of neutral phospholipids suitable for use in the present disclosure include, but are not limited to, dipalmitoylphosphatidylcholine (DPPC), distearoylphosphatidylcholine (DSPC), phosphocholine (DOPC), dimyristoylphosphatidylcholine (DMPC), phosphatidylcholine (PLPC), 1,2-distearoyl-sn-glycero-3-phosphocholine (DAPC), phosphatidylethanolamine (PE), egg phosphatidylcholine (EPC), dilauryloylphosphatidylcholine (DLPC), dimyristoylphosphatidylcholine (DMPC), 1-myristoyl-2-palmitoyl phosphatidylcholine (MPPC), 1-palmitoyl-2-myristoyl phosphatidylcholine (PMPC), 1-palmitoyl-2-stearoyl phosphatidylcholine (PSPC), 1,2-diarachidoyl-sn-glycero-3-phosphocholine (DBPC), 1-stearoyl-2-palmitoyl phosphatidylcholine (SPPC), 1,2-dieicosenoyl-sn-glycero-3-phosphocholine (DEPC), palmitoyloleoyl phosphatidylcholine (POPC), lysophosphatidyl choline, dioleoyl phosphatidylethanolamine (DOPE), dilinoleoylphosphatidylcholine distearoylphosphatidylethanolamine (DSPE), dimyristoyl phosphatidylethanolamine (DMPE), dipalmitoyl phosphatidylethanolamine (DPPE), palmitoyloleoyl phosphatidylethanolamine (POPE), lysophosphatidylethanolamine and combinations thereof. In certain embodiments, the neutral phospholipid may be selected from distearoylphosphatidylcholine (DSPC) and dimyristoyl phosphatidyl ethanolamine (DMPE), preferably distearoylphosphatidylcholine (DSPC).

“Helper lipids” include steroids, sterols, and alkyl resorcinols. Helper lipids suitable for use in the present disclosure include, but are not limited to, cholesterol, 5-heptadecylresorcinol, and cholesterol hemisuccinate. In certain embodiments, the helper lipid may be cholesterol or a derivative thereof, such as cholesterol hemisuccinate.

PEG lipids can affect the length of time the nanoparticles can exist in vivo (e.g., in the blood). PEG lipids may assist in the formulation process by, for example, reducing particle aggregation and controlling particle size. PEG lipids used herein may modulate pharmacokinetic properties of the LNPs. Typically, the PEG lipid comprises a lipid moiety and a polymer moiety based on PEG (sometimes referred to as poly(ethylene oxide)) (a PEG moiety). PEG lipids suitable for use in a lipid composition with a compound of Formula(I) or Formula (II) of the present disclosure and information about the biochemistry of such lipids can be found in Romberg et al., Pharmaceutical Research 25(1), 2008, pp. 55-71 and Hoekstra et al., Biochimica et Biophysica Acta 1660 (2004) 41-52. Additional suitable PEG lipids are disclosed, e.g., in WO 2015/095340 (p. 31, line 14 to p. 37, line 6), WO 2006/007712, and WO 2011/076807 (“stealth lipids”).

In some embodiments, the lipid moiety may be derived from diacylglycerol or diacylglycamide, including those comprising a dialkylglycerol or dialkylglycamide group having alkyl chain length independently comprising from about C4 to about C40 saturated or unsaturated carbon atoms, wherein the chain may comprise one or more functional groups such as, for example, an amide or ester. In some embodiments, the alkyl chain length comprises about C10 to C20. The dialkylglycerol or dialkylglycamide group can further comprise one or more substituted alkyl groups. The chain lengths may be symmetrical or asymmetric.

Unless otherwise indicated, the term “PEG” as used herein means any polyethylene glycol or other polyalkylene ether polymer, such as an optionally substituted linear or branched polymer of ethylene glycol or ethylene oxide. In certain embodiments, the PEG moiety is unsubstituted. Alternatively, the PEG moiety may be substituted, e.g., by one or more alkyl, alkoxy, acyl, hydroxy, or aryl groups. For example, the PEG moiety may comprise a PEG copolymer such as PEG-polyurethane or PEG-polypropylene (see, e.g., J. Milton Harris, Poly(ethylene glycol) chemistry: biotechnical and biomedical applications (1992)); alternatively, the PEG moiety may be a PEG homopolymer. In certain embodiments, the PEG moiety has a molecular weight of from about 130 to about 50,000, such as from about 150 to about 30,000, or even from about 150 to about 20,000. Similarly, the PEG moiety may have a molecular weight of from about 150 to about 15,000, from about 150 to about 10,000, from about 150 to about 6,000, or even from about 150 to about 5,000. In certain preferred embodiments, the PEG moiety has a molecular weight of from about 150 to about 4,000, from about 150 to about 3,000, from about 300 to about 3,000, from about 1,000 to about 3,000, or from about 1,500 to about 2,500.

In certain preferred embodiments, the PEG moiety is a “PEG-2K,” also termed “PEG 2000,” which has an average molecular weight of about 2,000 daltons. PEG-2K is represented herein by the following formula (II), wherein n is 45, meaning that the number averaged degree of polymerization comprises about 45 subunits

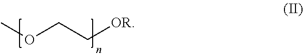

However, other PEG embodiments known in the art may be used, including, e.g., those where the number-averaged degree of polymerization comprises about 23 subunits (n=23), and/or 68 subunits (n=68). In some embodiments, n may range from about 30 to about 60. In some embodiments, n may range from about 35 to about 55. In some embodiments, n may range from about 40 to about 50. In some embodiments, n may range from about 42 to about 48. In some embodiments, n may be 45. In some embodiments, R may be selected from H, substituted alkyl, and unsubstituted alkyl. In some embodiments, R may be unsubstituted alkyl, such as methyl.

In any of the embodiments described herein, the PEG lipid may be selected from PEG-dilauroylglycerol, PEG-dimyristoylglycerol (PEG-DMG) (catalog #GM-020 from NOF, Tokyo, Japan), PEG-dipalmitoylglycerol, PEG-distearoylglycerol (PEG-DSPE) (catalog #DSPE-020CN, NOF, Tokyo, Japan), PEG-dilaurylglycamide, PEG-dimyristylglycamide, PEG-dipalmitoylglycamide, and PEG-distearoylglycamide, PEG-cholesterol (1-[8′-(Cholest-5-en-3[beta]-oxy)carboxamido-3′,6′-dioxaoctanyl]carbamoyl-[omega]-methyl-poly(ethylene glycol), PEG-DMB (3,4-ditetradecoxylbenzyl-[omega]-methyl-poly(ethylene glycol)ether), 1,2-dimyristoyl-sn-glycero-3-phosphoethanolamine-N-[methoxy(polyethylene glycol)-2000] (PEG2k-DMG), 1,2-distearoyl-sn-glycero-3-phosphoethanolamine-N-[methoxy(polyethylene glycol)-2000] (PEG2k-DSPE) (cat. #880120C from Avanti Polar Lipids, Alabaster, Ala., USA), 1,2-distearoyl-sn-glycerol, methoxypolyethylene glycol (PEG2k-DSG; GS-020, NOF Tokyo, Japan), poly(ethylene glycol)-2000-dimethacrylate (PEG2k-DMA), and 1,2-distearyloxypropyl-3-amine-N-[methoxy(polyethylene glycol)-2000](PEG2k-DSA). In certain such embodiments, the PEG lipid may be PEG2k-DMG. In some embodiments, the PEG lipid may be PEG2k-DSG. In other embodiments, the PEG lipid may be PEG2k-DSPE. In some embodiments, the PEG lipid may be PEG2k-DMA. In yet other embodiments, the PEG lipid may be PEG2k-C-DMA. In certain embodiments, the PEG lipid may be compound S027, disclosed in WO2016/010840 (paragraphs [00240] to [00244]). In some embodiments, the PEG lipid may be PEG2k-DSA. In other embodiments, the PEG lipid may be PEG2k-C11. In some embodiments, the PEG lipid may be PEG2k-C14. In some embodiments, the PEG lipid may be PEG2k-C16. In some embodiments, the PEG lipid may be PEG2k-C18.

Cationic lipids suitable for use in a lipid composition of the invention include, but are not limited to, N,N-dioleyl-N,N-dimethylammonium chloride (DODAC), N,N-distearyl-N,N-dimethylammonium bromide (DDAB), N-(1-(2,3-dioleoyloxy)propyl)-N,N,N-trimethylammonium chloride (DOTAP), 1,2-Dioleoyl-3-Dimethylammonium-propane (DODAP), N-(1-(2,3-dioleyloxy)propyl)-N,N,N-trimethylammonium chloride (DOTMA), 1,2-Dioleoylcarbamyl-3-Dimethylammonium-propane (DOCDAP), 1,2-Dilineoyl-3-Dimethylammonium-propane (DLINDAP), dilauryl(C12:0) trimethyl ammonium propane (DLTAP), Dioctadecylamidoglycyl spermine (DOGS), DC-Choi, Dioleoyloxy-N-[2-(sperminecarboxamido)ethyl]-N,N-dimethyl-1-propanaminiumtrifluoroacetate (DOSPA), 1,2-Dimyristyloxypropyl-3-dimethyl-hydroxyethyl ammonium bromide (DMRIE), 3-Dimethylamino-2-(Cholest-5-en-3-beta-oxybutan-4-oxy)-1-(cis,cis-9,12-octadecadienoxy)propane (CLinDMA), N,N-dimethyl-2,3-dioleyloxy)propylamine (DODMA), 2-[5′-(cholest-5-en-3[beta]-oxy)-3′-oxapentoxy)-3-dimethyl-1-(cis,cis-9′,1-2′-octadecadienoxy) propane (CpLinDMA), N,N-Dimethyl-3,4-dioleyloxybenzylamine (DMOBA), and 1,2-N,N′-Dioleylcarbamyl-3-dimethylaminopropane (DOcarbDAP). In one embodiment the cationic lipid is DOTAP or DLTAP.

Anionic lipids suitable for use in the present invention include, but are not limited to, phosphatidylglycerol, cardiolipin, diacylphosphatidylserine, diacylphosphatidic acid, N-dodecanoyl phosphatidyl ethanolamine, N-succinyl phosphatidylethanolamine, N-glutaryl phosphatidylethanolamine cholesterol hemisuccinate (CHEMS), and lysylphosphatidylglycerol.

Lipid Compositions

The present invention provides a lipid composition comprising at least one compound of Formula(I) or Formula (II) or a salt thereof (e.g., a pharmaceutically acceptable salt thereof) and at least one other lipid component. Such compositions can also contain a biologically active agent, optionally in combination with one or more other lipid components. In some embodiments, the lipid compositions comprise a lipid component and an aqueous component comprising a biologically active agent.

In one embodiment, the lipid composition comprises a compound of Formula(I) or Formula (II), or a pharmaceutically acceptable salt thereof, and at least one other lipid component. In another embodiment, the lipid composition further comprises a biologically active agent, optionally in combination with one or more other lipid components. In another embodiment the lipid composition is in the form of a liposome. In another embodiment the lipid composition is in the form of a lipid nanoparticle (LNP). In another embodiment the lipid composition is suitable for delivery to the liver.

In one embodiment, the lipid composition comprises a compound of Formula(I) or Formula (II), or a pharmaceutically acceptable salt thereof, and another lipid component. Such other lipid components include, but are not limited to, neutral lipids, helper lipids, PEG lipids, cationic lipids, and anionic lipids. In certain embodiments, the lipid composition comprises a compound of Formula(I) or Formula (II), or a pharmaceutically acceptable salt thereof, and a neutral lipid, e.g. DSPC, optionally with one or more additional lipid components. In another embodiment, the lipid composition comprises a compound of Formula(I) or Formula (II), or a pharmaceutically acceptable salt thereof, and a helper lipid, e.g. cholesterol, optionally with one or more additional lipid components. In further embodiment, the lipid composition comprises a compound of Formula(I) or Formula (II), or a pharmaceutically acceptable salt thereof, and a PEG lipid, optionally with one or more additional lipid components. In further embodiment, the lipid composition comprises a compound of Formula(I) or Formula (II), or a pharmaceutically acceptable salt thereof, and a cationic lipid, optionally with one or more additional lipid components. In further embodiment, the lipid composition comprises a compound of Formula(I) or Formula (II), or a pharmaceutically acceptable salt thereof, and an anionic lipid, optionally with one or more additional lipid components. In a sub-embodiment, the lipid composition comprises a compound of Formula(I) or Formula (II), or a pharmaceutically acceptable salt thereof, a helper lipid, and a PEG lipid, optionally with a neutral lipid. In a further sub-embodiment, the lipid composition comprises a compound of Formula(I) or Formula (II), or a pharmaceutically acceptable salt thereof, a helper lipid, a PEG lipid, and a neutral lipid.

Compositions containing lipids of Formula(I) or Formula (II), or a pharmaceutically acceptable salt thereof, or lipid compositions thereof may be in various forms, including, but not limited to, particle forming delivery agents including microparticles, nanoparticles and transfection agents that are useful for delivering various molecules to cells. Specific compositions are effective at transfecting or delivering biologically active agents. Preferred biologically active agents are RNAs and DNAs. In further embodiments, the biologically active agent is chosen from mRNA, gRNA, and DNA. In certain embodiments, the cargo includes an mRNA encoding an RNA-guided DNA-binding agent (e.g. a Cas nuclease, a Class 2 Cas nuclease, or Cas9), and a gRNA or a nucleic acid encoding a gRNA, or a combination of mRNA and gRNA.

Exemplary compounds of Formula (I) for use in the above lipid compositions are given in the Examples. In certain embodiments, the compound of Formula (I) is Compound 1. In certain embodiments, the compound of Formula (I) is Compound 2. In certain embodiments, the compound of Formula (I) is Compound 3. In certain embodiments, the compound of Formula (I) is Compound 4. In certain embodiments, the compound of Formula (I) is Compound 5. In certain embodiments, the compound of Formula (I) is Compound 6. In certain embodiments, the compound of Formula (I) is Compound 7. In certain embodiments, the compound of Formula (I) is Compound 8. In certain embodiments, the compound of Formula (I) is Compound 9. In certain embodiments, the compound of Formula (I) is Compound 10. In certain embodiments, the compound of Formula (I) is Compound 11. In certain embodiments, the compound of Formula (I) is Compound 12. In certain embodiments, the compound of Formula (I) is Compound 13. In certain embodiments, the compound of Formula (I) is Compound 14. In certain embodiments, the compound of Formula (I) is Compound 15. In certain embodiments, the compound of Formula (I) is Compound 16. In certain embodiments, the compound of Formula (I) is Compound 17. In certain embodiments, the compound of Formula (I) is Compound 20. In certain embodiments, the compound of Formula (I) is Compound 21. In certain embodiments, the compound of Formula (I) is Compound 22. In certain embodiments, the compound of Formula (I) is Compound 23. In certain embodiments, the compound of Formula (I) is Compound 24. In certain embodiments, the compound of Formula (I) is Compound 25. In certain embodiments, the compound of Formula (I) is Compound 27. In certain embodiments, the compound of Formula (I) is Compound 28. In certain embodiments, the compound of Formula (I) is Compound 29. In certain embodiments, the compound of Formula (I) is Compound 30. In certain embodiments, the compound of Formula (I) is Compound 31. In certain embodiments, the compound of Formula (I) is Compound 32. In certain embodiments, the compound of Formula (I) is Compound 33. In certain embodiments, the compound of Formula (I) is Compound 34. In certain embodiments, the compound of Formula (I) is Compound 35. In certain embodiments, the compound of Formula (I) is Compound 36. In certain embodiments, the compound of Formula (I) is Compound 37. In certain embodiments, the compound of Formula (I) is Compound 38. In certain embodiments, the compound of Formula (I) is Compound 39. In certain embodiments, the compound of Formula (I) is Compound 40. In certain embodiments, the compound of Formula (I) is Compound 41. In certain embodiments, the compound of Formula (I) is Compound 42. In certain embodiments, the compound of Formula (I) is Compound 43. In certain embodiments, the compound of Formula (I) is Compound 44. In certain embodiments, the compound of Formula (I) is Compound 45. In certain embodiments, the compound of Formula (I) is Compound 46. In certain embodiments, the compound of Formula (I) is Compound 47. In certain embodiments, the compound of Formula (I) is Compound 48. In certain embodiments, the compound of Formula (I) is Compound 49. In certain embodiments, the compound of Formula (I) is Compound 50. In certain embodiments, the compound is a compound selected from the compounds in Table 1, provided the compound is not Compound 18, Compound 19, or Compound 26.

### LNP Compositions

The lipid compositions may be provided as LNP compositions. Lipid nanoparticles may be, e.g., microspheres (including unilamellar and multilamellar vesicles, e.g. “liposomes”-lamellar phase lipid bilayers that, in some embodiments are substantially spherical, and, in more particular embodiments can comprise an aqueous core, e.g., comprising a substantial portion of RNA molecules), a dispersed phase in an emulsion, micelles or an internal phase in a suspension.

The LNPs have a size of about 1 to about 1,000 nm, about 10 to about 500 nm, about 20 to about 500 nm, in a sub-embodiment about 50 to about 400 nm, in a sub-embodiment about 50 to about 300 nm, in a sub-embodiment about 50 to about 200 nm, and in a sub-embodiment about 50 to about 150 nm, and in another sub-embodiment about 60 to about 120 nm. Preferably, the LNPs have a size from about 60 nm to about 100 nm. The average sizes (diameters) of the fully formed LNP, may be measured by dynamic light scattering on a Malvern Zetasizer. The LNP sample is diluted in phosphate buffered saline (PBS) so that the count rate is approximately 200-400 kcps. The data is presented as a weighted average of the intensity measure.

Embodiments of the present disclosure provide lipid compositions described according to the respective molar ratios of the component lipids in the composition. All mol-% numbers are given as a fraction of the lipid component of the lipid composition or, more specifically, the LNP compositions. In certain embodiments, the mol-% of the compound of Formula(I) or Formula (II) may be from about 30 mol-% to about 70 mol-%. In certain embodiments, the mol-% of the compound of Formula(I) or Formula (II) may at least 30 mol-%, at least 40 mol-%, at least 50 mol-%, or at least 60 mol-%.

In certain embodiments, the mol-% of the neutral lipid may be from about 0 mol-% to about 30 mol-%. In certain embodiments, the mol-% of the neutral lipid may be from about 0 mol-% to about 20 mol-%. In certain embodiments, the mol-% of the neutral lipid may be about 9 mol-%.

In certain embodiments, the mol-% of the helper lipid may be from about 0 mol-% to about 80 mol-%. In certain embodiments, the mol-% of the helper lipid may be from about 20 mol-% to about 60 mol-%. In certain embodiments, the mol-% of the helper lipid may be from about 30 mol-% to about 50 mol-%. In certain embodiments, the mol-% of the helper lipid may be from 30 mol-% to about 40 mol-% or from about 35% mol-% to about 45 mol-%. In certain embodiments, the mol-% of the helper lipid is adjusted based on compound of Formula(I) or Formula (II), neutral lipid, and/or PEG lipid concentrations to bring the lipid component to 100 mol-%.

In certain embodiments, the mol-% of the PEG lipid may be from about 1 mol-% to about 10 mol-%. In certain embodiments, the mol-% of the PEG lipid may be from about 1 mol-% to about 4 mol-%. In certain embodiments, the mol-% of the PEG lipid may be about 1 mol-% to about 2 mol-%. In certain embodiments, the mol-% of the PEG lipid may be about 1.5 mol-%.

In various embodiments, an LNP composition comprises a compound of Formula(I) or Formula (II) or a salt thereof (such as a pharmaceutically acceptable salt thereof (e.g., as disclosed herein)), a neutral lipid (e.g., DSPC), a helper lipid (e.g., cholesterol), and a PEG lipid (e.g., PEG2k-DMG). In some embodiments, an LNP composition comprises a compound of Formula(I) or Formula (II) or a pharmaceutically acceptable salt thereof (e.g., as disclosed herein), DSPC, cholesterol, and a PEG lipid. In some such embodiments, the LNP composition comprises a PEG lipid comprising DMG, such as PEG2k-DMG. In certain preferred embodiments, an LNP composition comprises a compound of Formula(I) or Formula (II) or a pharmaceutically acceptable salt thereof, cholesterol, DSPC, and PEG2k-DMG.

In certain embodiments, the lipid compositions, such as LNP compositions, comprise a lipid component and a nucleic acid component, e.g. an RNA component and the molar ratio of compound of Formula(I) or Formula (II) to nucleic acid can be measured. Embodiments of the present disclosure also provide lipid compositions having a defined molar ratio between the positively charged amine groups of pharmaceutically acceptable salts of the compounds of Formula(I) or Formula (II) (N) and the negatively charged phosphate groups (P) of the nucleic acid to be encapsulated. This may be mathematically represented by the equation N/P. In some embodiments, a lipid composition, such as an LNP composition, may comprise a lipid component that comprises a compound of Formula(I) or Formula (II) or a pharmaceutically acceptable salt thereof; and a nucleic acid component, wherein the N/P ratio is about 3 to 10. In some embodiments, an LNP composition may comprise a lipid component that comprises a compound of Formula(I) or Formula (II) or a pharmaceutically acceptable salt thereof; and an RNA component, wherein the N/P ratio is about 3 to 10. For example, the N/P ratio may be about 4-7. Alternatively, the N/P ratio may about 6, e.g., 6±1, or 6±0.5.

In some embodiments, the aqueous component comprises a biologically active agent. In some embodiments, the aqueous component comprises a polypeptide, optionally in combination with a nucleic acid. In some embodiments, the aqueous component comprises a nucleic acid, such as an RNA. In some embodiments, the aqueous component is a nucleic acid component. In some embodiments, the nucleic acid component comprises DNA and it can be called a DNA component. In some embodiments, the nucleic acid component comprises RNA. In some embodiments, the aqueous component, such as an RNA component may comprise an mRNA, such as an mRNA encoding an RNA-guided DNA binding agent. In some embodiments, the RNA-guided DNA binding agent is a Cas nuclease. In certain embodiments, aqueous component may comprise an mRNA that encodes Cas9. In certain embodiments, the aqueous component may comprise a gRNA. In some compositions comprising an mRNA encoding an RNA-guided DNA binding agent, the composition further comprises a gRNA nucleic acid, such as a gRNA. In some embodiments, the aqueous component comprises an RNA-guided DNA binding agent and a gRNA. In some embodiments, the aqueous component comprises a Cas nuclease mRNA and a gRNA. In some embodiments, the aqueous component comprises a Class 2 Cas nuclease mRNA and a gRNA.

In certain embodiments, a lipid composition, such as an LNP composition, may comprise an mRNA encoding a Cas nuclease such as a Class 2 Cas nuclease, a compound of Formula(I) or Formula (II) or a pharmaceutically acceptable salt thereof, a helper lipid, optionally a neutral lipid, and a PEG lipid. In certain compositions comprising an mRNA encoding a Cas nuclease such as a Class 2 Cas nuclease, the helper lipid is cholesterol. In other compositions comprising an mRNA encoding a Cas nuclease such as a Class 2 Cas nuclease, the neutral lipid is DSPC. In additional embodiments comprising an mRNA encoding a Cas nuclease such as a Class 2 Cas nuclease, e.g. Cas9, the PEG lipid is PEG2k-DMG. In specific compositions comprising an mRNA encoding a Cas nuclease such as a Class 2 Cas nuclease, and a compound of Formula(I) or Formula (II) or a pharmaceutically acceptable salt thereof. In certain compositions, the composition further comprises a gRNA, such as a dgRNA or an sgRNA.

In some embodiments, a lipid composition, such as an LNP composition, may comprise a gRNA. In certain embodiments, a composition may comprise a compound of Formula(I) or Formula (II) or a pharmaceutically acceptable salt thereof, a gRNA, a helper lipid, optionally a neutral lipid, and a PEG lipid. In certain LNP compositions comprising a gRNA, the helper lipid is cholesterol. In some compositions comprising a gRNA, the neutral lipid is DSPC. In additional embodiments comprising a gRNA, the PEG lipid is PEG2k-DMG. In certain compositions, the gRNA is selected from dgRNA and sgRNA.

In certain embodiments, a lipid composition, such as an LNP composition, comprises an mRNA encoding an RNA-guided DNA binding agent and a gRNA, which may be an sgRNA, in an aqueous component and a compound of Formula(I) or Formula (II) in a lipid component. For example, an LNP composition may comprise a compound of Formula(I) or Formula (II) or a pharmaceutically acceptable salt thereof, an mRNA encoding a Cas nuclease, a gRNA, a helper lipid, a neutral lipid, and a PEG lipid. In certain compositions comprising an mRNA encoding a Cas nuclease and a gRNA, the helper lipid is cholesterol. In some compositions comprising an mRNA encoding a Cas nuclease and a gRNA, the neutral lipid is DSPC. In additional embodiments comprising an mRNA encoding a Cas nuclease and a gRNA, the PEG lipid is PEG2k-DMG.

In certain embodiments, the lipid compositions, such as LNP compositions include an RNA-guided DNA binding agent, such as a Class 2 Cas mRNA and at least one gRNA. In certain embodiments, the LNP composition includes a ratio of gRNA to RNA-guided DNA binding agent mRNA, such as Class 2 Cas nuclease mRNA of about 1:1 or about 1:2. In some embodiments, the ratio is from about 25:1 to about 1:25, from about 10:1 to about 1:10, from about 8:1 to about 1:8, from about 4:1 to about 1:4, or from about 2:1 to about 1:2.

The lipid compositions disclosed herein, such as LNP compositions, may include a template nucleic acid, e.g., a DNA template. The template nucleic acid may be delivered with, or separately from the lipid compositions comprising a compound of Formula(I) or Formula (II) or a pharmaceutically acceptable salt thereof, including as LNP compositions. In some embodiments, the template nucleic acid may be single- or double-stranded, depending on the desired repair mechanism. The template may have regions of homology to the target DNA, e.g. within the target DNA sequence, and/or to sequences adjacent to the target DNA.

In some embodiments, LNPs are formed by mixing an aqueous RNA solution with an organic solvent-based lipid solution. Suitable solutions or solvents include or may contain: water, PBS, Tris buffer, NaCl, citrate buffer, acetate buffer, ethanol, chloroform, diethylether, cyclohexane, tetrahydrofuran, methanol, isopropanol. For example, the organic solvent may be 100% ethanol. A pharmaceutically acceptable buffer, e.g., for in vivo administration of LNPs, may be used. In certain embodiments, a buffer is used to maintain the pH of the composition comprising LNPs at or above pH 6.5. In certain embodiments, a buffer is used to maintain the pH of the composition comprising LNPs at or above pH 7.0. In certain embodiments, the composition has a pH ranging from about 7.2 to about 7.7. In additional embodiments, the composition has a pH ranging from about 7.3 to about 7.7 or ranging from about 7.4 to about 7.6. In further embodiments, the composition has a pH of about 7.2, 7.3, 7.4, 7.5, 7.6, or 7.7. The pH of a composition may be measured with a micro pH probe. In certain embodiments, a cryoprotectant is included in the composition. Non-limiting examples of cryoprotectants include sucrose, trehalose, glycerol, DMSO, and ethylene glycol. Exemplary compositions may include up to 10% cryoprotectant, such as, for example, sucrose. In certain embodiments, the composition may comprise tris saline sucrose (TSS). In certain embodiments, the LNP composition may include about 1, 2, 3, 4, 5, 6, 7, 8, 9, or 10% cryoprotectant. In certain embodiments, the LNP composition may include about 1, 2, 3, 4, 5, 6, 7, 8, 9, or 10% sucrose. In some embodiments, the LNP composition may include a buffer. In some embodiments, the buffer may comprise a phosphate buffer (PBS), a Tris buffer, a citrate buffer, and mixtures thereof. In certain exemplary embodiments, the buffer comprises NaCl. In certain embodiments, the buffer lacks NaCl. Exemplary amounts of NaCl may range from about 20 mM to about 45 mM. Exemplary amounts of NaCl may range from about 40 mM to about 50 mM. In some embodiments, the amount of NaCl is about 45 mM. In some embodiments, the buffer is a Tris buffer. Exemplary amounts of Tris may range from about 20 mM to about 60 mM. Exemplary amounts of Tris may range from about 40 mM to about 60 mM. In some embodiments, the amount of Tris is about 50 mM. In some embodiments, the buffer comprises NaCl and Tris. Certain exemplary embodiments of the LNP compositions contain 5% sucrose and 45 mM NaCl in Tris buffer. In other exemplary embodiments, compositions contain sucrose in an amount of about 5% w/v, about 45 mM NaCl, and about 50 mM Tris at pH 7.5. The salt, buffer, and cryoprotectant amounts may be varied such that the osmolality of the overall composition is maintained. For example, the final osmolality may be maintained at less than 450 mOsm/L. In further embodiments, the osmolality is between 350 and 250 mOsm/L. Certain embodiments have a final osmolality of 300+/−20 mOsm/L or 310+/−40 mOsm/L.

In some embodiments, microfluidic mixing, T-mixing, or cross-mixing of the aqueous RNA solution and the lipid solution in an organic solvent is used. In certain aspects, flow rates, junction size, junction geometry, junction shape, tube diameter, solutions, and/or RNA and lipid concentrations may be varied. LNPs or LNP compositions may be concentrated or purified, e.g., via dialysis, centrifugal filter, tangential flow filtration, or chromatography. The LNPs may be stored as a suspension, an emulsion, or a lyophilized powder, for example. In some embodiments, an LNP composition is stored at 2-8° C., in certain aspects, the LNP compositions are stored at room temperature. In additional embodiments, an LNP composition is stored frozen, for example at −20° C. or −80° C. In other embodiments, an LNP composition is stored at a temperature ranging from about 0° C. to about −80° C. Frozen LNP compositions may be thawed before use, for example on ice, at room temperature, or at 25° C.

The LNPs may be, e.g., microspheres (including unilamellar and multilamellar vesicles, e.g., “liposomes”—lamellar phase lipid bilayers that, in some embodiments, are substantially spherical—and, in more particular embodiments, can comprise an aqueous core, e.g., comprising a substantial portion of RNA molecules), a dispersed phase in an emulsion, micelles, or an internal phase in a suspension.

Preferred lipid compositions, such as LNP compositions, are biodegradable, in that they do not accumulate to cytotoxic levels in vivo at a therapeutically effective dose. In some embodiments, the compositions do not cause an innate immune response that leads to substantial adverse effects at a therapeutic dose level. In some embodiments, the compositions provided herein do not cause toxicity at a therapeutic dose level.

In some embodiments, the LNPs disclosed herein have a polydispersity index (PDI) that may range from about 0.005 to about 0.75. In some embodiments, the LNP have a PDI that may range from about 0.01 to about 0.5. In some embodiments, the LNP have a PDI that may range from about zero to about 0.4. In some embodiments, the LNP have a PDI that may range from about zero to about 0.35. In some embodiments, the LNP have a PDI that may range from about zero to about 0.35. In some embodiments, the LNP PDI may range from about zero to about 0.3. In some embodiments, the LNP have a PDI that may range from about zero to about 0.25. In some embodiments, the LNP PDI may range from about zero to about 0.2. In some embodiments, the LNP have a PDI that may be less than about 0.08, 0.1, 0.15, 0.2, or 0.4.

The LNPs disclosed herein have a size (e.g. Z-average diameter) of about 1 to about 250 nm. In some embodiments, the LNPs have a size of about 10 to about 200 nm. In further embodiments, the LNPs have a size of about 20 to about 150 nm. In some embodiments, the LNPs have a size of about 50 to about 150 nm. In some embodiments, the LNPs have a size of about 50 to about 100 nm. In some embodiments, the LNPs have a size of about 50 to about 120 nm. In some embodiments, the LNPs have a size of about 60 to about 100 nm. In some embodiments, the LNPs have a size of about 75 to about 150 nm. In some embodiments, the LNPs have a size of about 75 to about 120 nm. In some embodiments, the LNPs have a size of about 75 to about 100 nm. Unless indicated otherwise, all sizes referred to herein are the average sizes (diameters) of the fully formed nanoparticles, as measured by dynamic light scattering on a Malvern Zetasizer. The nanoparticle sample is diluted in phosphate buffered saline (PBS) so that the count rate is approximately 200-400 kcps. The data is presented as a weighted-average of the intensity measure (Z-average diameter).

In some embodiments, the LNPs are formed with an average encapsulation efficiency ranging from about 50% to about 100%. In some embodiments, the LNPs are formed with an average encapsulation efficiency ranging from about 50% to about 95%. In some embodiments, the LNPs are formed with an average encapsulation efficiency ranging from about 70% to about 90%. In some embodiments, the LNPs are formed with an average encapsulation efficiency ranging from about 90% to about 100%. In some embodiments, the LNPs are formed with an average encapsulation efficiency ranging from about 75% to about 95%.

### Cargo

The cargo delivered via LNP composition may be a biologically active agent. In certain embodiments, the cargo is or comprises one or more biologically active agent, such as mRNA, guide RNA, nucleic acid, RNA-guided DNA-binding agent, expression vector, template nucleic acid, antibody (e.g., monoclonal, chimeric, humanized, nanobody, and fragments thereof etc.), cholesterol, hormone, peptide, protein, chemotherapeutic and other types of antineoplastic agent, low molecular weight drug, vitamin, co-factor, nucleoside, nucleotide, oligonucleotide, enzymatic nucleic acid, antisense nucleic acid, triplex forming oligonucleotide, antisense DNA or RNA composition, chimeric DNA:RNA composition, allozyme, aptamer, ribozyme, decoys and analogs thereof, plasmid and other types of vectors, and small nucleic acid molecule, RNAi agent, short interfering nucleic acid (siNA), short interfering RNA (siRNA), double-stranded RNA (dsRNA), micro-RNA (miRNA), short hairpin RNA (shRNA) and “self-replicating RNA” (encoding a replicase enzyme activity and capable of directing its own replication or amplification in vivo) molecules, peptide nucleic acid (PNA), a locked nucleic acid ribonucleotide (LNA), morpholino nucleotide, threose nucleic acid (TNA), glycol nucleic acid (GNA), sisiRNA (small internally segmented interfering RNA), and iRNA (asymmetrical interfering RNA). The above list of biologically active agents is exemplary only, and is not intended to be limiting. Such compounds may be purified or partially purified, and may be naturally occurring or synthetic, and may be chemically modified.

The cargo delivered via LNP composition may be an RNA, such as an mRNA molecule encoding a protein of interest. For example, an mRNA for expressing a protein such as green fluorescent protein (GFP), an RNA-guided DNA-binding agent, or a Cas nuclease is included. LNP compositions that include a Cas nuclease mRNA, for example a Class 2 Cas nuclease mRNA that allows for expression in a cell of a Class 2 Cas nuclease such as a Cas9 or Cpf1 protein are provided. Further, the cargo may contain one or more guide RNAs or nucleic acids encoding guide RNAs. A template nucleic acid, e.g., for repair or recombination, may also be included in the composition or a template nucleic acid may be used in the methods described herein. In a sub-embodiment, the cargo comprises an mRNA that encodes a Streptococcus pyogenes Cas9, optionally and an S. pyogenes gRNA. In a further sub-embodiment, the cargo comprises an mRNA that encodes a Neisseria meningitidis Cas9, optionally and an nme gRNA.

“mRNA” refers to a polynucleotide and comprises an open reading frame that can be translated into a polypeptide (i.e., can serve as a substrate for translation by a ribosome and amino-acylated tRNAs). mRNA can comprise a phosphate-sugar backbone including ribose residues or analogs thereof, e.g., 2′-methoxy ribose residues. In some embodiments, the sugars of an mRNA phosphate-sugar backbone consist essentially of ribose residues, 2′-methoxy ribose residues, or a combination thereof. In general, mRNAs do not contain a substantial quantity of thymidine residues (e.g., 0 residues or fewer than 30, 20, 10, 5, 4, 3, or 2 thymidine residues; or less than 10%, 9%, 8%, 7%, 6%, 5%, 4%, 4%, 3%, 2%, 1%, 0.5%, 0.2%, or 0.1% thymidine content). An mRNA can contain modified uridines at some or all of its uridine positions.

### CRISPR/Cas Cargo

In certain embodiments, the disclosed compositions comprise an mRNA encoding an RNA-guided DNA-binding agent, such as a Cas nuclease. In particular embodiments, the disclosed compositions comprise an mRNA encoding a Class 2 Cas nuclease, such as S. pyogenes Cas9.

As used herein, an “RNA-guided DNA binding agent” means a polypeptide or complex of polypeptides having RNA and DNA binding activity, or a DNA-binding subunit of such a complex, wherein the DNA binding activity is sequence-specific and depends on the sequence of the RNA. Exemplary RNA-guided DNA binding agents include Cas cleavases/nickases and inactivated forms thereof (“dCas DNA binding agents”). “Cas nuclease”, as used herein, encompasses Cas cleavases, Cas nickases, and dCas DNA binding agents. Cas cleavases/nickases and dCas DNA binding agents include a Csm or Cmr complex of a type III CRISPR system, the Cas10, Csm1, or Cmr2 subunit thereof, a Cascade complex of a type I CRISPR system, the Cas3 subunit thereof, and Class 2 Cas nucleases. As used herein, a “Class 2 Cas nuclease” is a single-chain polypeptide with RNA-guided DNA binding activity. Class 2 Cas nucleases include Class 2 Cas cleavases/nickases (e.g., H840A, D10A, or N863 A variants), which further have RNA-guided DNA cleavases or nickase activity, and Class 2 dCas DNA binding agents, in which cleavase/nickase activity is inactivated. Class 2 Cas nucleases include, for example, Cas9, Cpf1, C2c1, C2c2, C2c3, HF Cas9 (e.g., N497A, R661A, Q695A, Q926A variants), HypaCas9 (e.g., N692A, M694A, Q695A, H698A variants), eSPCas9(1.0) (e.g, K810A, K1003A, R1060A variants), and eSPCas9(1.1) (e.g., K848A, K1003A, R1060A variants) proteins and modifications thereof. Cpf1 protein, Zetsche et al., Cell, 163: 1-13 (2015), is homologous to Cas9, and contains a RuvC-like nuclease domain. Cpf1 sequences of Zetsche are incorporated by reference in their entirety. See, e.g., Zetsche, Tables SI and S3. See, e.g, Makarova et al., Nat Rev Microbiol, 13(11): 722-36 (2015); Shmakov et al., Molecular Cell, 60:385-397 (2015).

As used herein, “ribonucleoprotein” (RNP) or “RNP complex” refers to a guide RNA together with an RNA-guided DNA binding agent, such as a Cas nuclease, e.g., a Cas cleavase, Cas nickase, or dCas DNA binding agent (e.g., Cas9). In some embodiments, the guide RNA guides the RNA-guided DNA binding agent such as Cas9 to a target sequence, and the guide RNA hybridizes with and the agent binds to the target sequence; in cases where the agent is a cleavase or nickase, binding can be followed by cleaving or nicking.

In some embodiments of the present disclosure, the cargo for the LNP composition includes at least one guide RNA comprising guide sequences that direct an RNA-guided DNA binding agent, which can be a nuclease (e.g., a Cas nuclease such as Cas9), to a target DNA. The gRNA may guide the Cas nuclease or Class 2 Cas nuclease to a target sequence on a target nucleic acid molecule. In some embodiments, a gRNA binds with and provides specificity of cleavage by a Class 2 Cas nuclease. In some embodiments, the gRNA and the Cas nuclease may form a ribonucleoprotein (RNP), e.g, a CRISPR/Cas complex such as a CRISPR/Cas9 complex. In some embodiments, the CRISPR/Cas complex may be a Type-II CRISPR/Cas9 complex. In some embodiments, the CRISPR/Cas complex may be a Type-V CRISPR/Cas complex, such as a Cpf1/guide RNA complex. Cas nucleases and cognate gRNAs may be paired. The gRNA scaffold structures that pair with each Class 2 Cas nuclease vary with the specific CRISPR/Cas system.

“Guide RNA”, “gRNA”, and simply “guide” are used herein interchangeably to refer to either a crRNA (also known as CRISPR RNA), or the combination of a crRNA and a trRNA (also known as tracrRNA). Guide RNAs can include modified RNAs as described herein. The crRNA and trRNA may be associated as a single RNA molecule (single guide RNA, sgRNA) or in two separate RNA molecules (dual guide RNA, dgRNA). “Guide RNA” or “gRNA” refers to each type. The trRNA may be a naturally-occurring sequence, or a trRNA sequence with modifications or variations compared to naturally-occurring sequences.

As used herein, a “guide sequence” refers to a sequence within a guide RNA that is complementary to a target sequence and functions to direct a guide RNA to a target sequence for binding or modification (e.g., cleavage) by an RNA-guided DNA binding agent. A “guide sequence” may also be referred to as a “targeting sequence,” or a “spacer sequence.” A guide sequence can be 20 base pairs in length, e.g., in the case of Streptococcus pyogenes (i.e., Spy Cas9) and related Cas9 homologs/orthologs. Shorter or longer sequences can also be used as guides, e.g, 15-, 16-, 17-, 18-, 19-, 21-, 22-, 23-, 24-, or 25-nucleotides in length. In some embodiments, the target sequence is in a gene or on a chromosome, for example, and is complementary to the guide sequence. In some embodiments, the degree of complementarity or identity between a guide sequence and its corresponding target sequence may be about or at least 75%, 80%, 85%, 90%, 95%, 96%, 97%, 98%, 99%, or 100%. In some embodiments, the guide sequence and the target region may be 100% complementary or identical over a region of at least 15, 16, 17, 18, 19, or 20 contiguous nucleotides. In other embodiments, the guide sequence and the target region may contain at least one mismatch. For example, the guide sequence and the target sequence may contain 1, 2, 3, or 4 mismatches, where the total length of the target sequence is at least 17, 18, 19, 20 or more base pairs. In some embodiments, the guide sequence and the target region may contain 1-4 mismatches where the guide sequence comprises at least 17, 18, 19, 20 or more nucleotides. In some embodiments, the guide sequence and the target region may contain 1, 2, 3, or 4 mismatches where the guide sequence comprises 20 nucleotides.

Target sequences for RNA-guided DNA binding proteins such as Cas proteins include both the positive and negative strands of genomic DNA (i.e., the sequence given and the sequence's reverse compliment), as a nucleic acid substrate for a Cas protein is a double stranded nucleic acid. Accordingly, where a guide sequence is said to be “complementary to a target sequence”, it is to be understood that the guide sequence may direct a guide RNA to bind to the reverse complement of a target sequence. Thus, in some embodiments, where the guide sequence binds the reverse complement of a target sequence, the guide sequence is identical to certain nucleotides of the target sequence (e.g., the target sequence not including the PAM) except for the substitution of U for T in the guide sequence.

The length of the targeting sequence may depend on the CRISPR/Cas system and components used. For example, different Class 2 Cas nucleases from different bacterial species have varying optimal targeting sequence lengths. Accordingly, the targeting sequence may comprise 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or more than 50 nucleotides in length. In some embodiments, the targeting sequence length is 0, 1, 2, 3, 4, or 5 nucleotides longer or shorter than the guide sequence of a naturally-occurring CRISPR/Cas system. In certain embodiments, the Cas nuclease and gRNA scaffold will be derived from the same CRISPR/Cas system. In some embodiments, the targeting sequence may comprise or consist of 18-24 nucleotides. In some embodiments, the targeting sequence may comprise or consist of 19-21 nucleotides. In some embodiments, the targeting sequence may comprise or consist of 20 nucleotides.

In some embodiments, the sgRNA is a “Cas9 sgRNA” capable of mediating RNA-guided DNA cleavage by a Cas9 protein. In some embodiments, the sgRNA is a “Cpf1 sgRNA” capable of mediating RNA-guided DNA cleavage by a Cpf1 protein. In certain embodiments, the gRNA comprises a crRNA and tracr RNA sufficient for forming an active complex with a Cas9 protein and mediating RNA-guided DNA cleavage. In certain embodiments, the gRNA comprises a crRNA sufficient for forming an active complex with a Cpf1 protein and mediating RNA-guided DNA cleavage. See Zetsche 2015.

Certain embodiments of the invention also provide nucleic acids, e.g., expression cassettes, encoding the gRNA described herein. A “guide RNA nucleic acid” is used herein to refer to a guide RNA (e.g. an sgRNA or a dgRNA) and a guide RNA expression cassette, which is a nucleic acid that encodes one or more guide RNAs.

Modified RNAs

In certain embodiments, the lipid compositions, such as LNP compositions comprise modified nucleic acids, including modified RNAs.

Modified nucleosides or nucleotides can be present in an RNA, for example a gRNA or mRNA. A gRNA or mRNA comprising one or more modified nucleosides or nucleotides, for example, is called a “modified” RNA to describe the presence of one or more non-naturally and/or naturally occurring components or configurations that are used instead of or in addition to the canonical A, G, C, and U residues. In some embodiments, a modified RNA is synthesized with a non-canonical nucleoside or nucleotide, here called “modified.”

Modified nucleosides and nucleotides can include one or more of: (i) alteration, e.g., replacement, of one or both of the non-linking phosphate oxygens and/or of one or more of the linking phosphate oxygens in the phosphodiester backbone linkage (an exemplary backbone modification); (ii) alteration, e.g., replacement, of a constituent of the ribose sugar, e.g., of the 2′ hydroxyl on the ribose sugar (an exemplary sugar modification); (iii) wholesale replacement of the phosphate moiety with “dephospho” linkers (an exemplary backbone modification); (iv) modification or replacement of a naturally occurring nucleobase, including with a non-canonical nucleobase (an exemplary base modification); (v) replacement or modification of the ribose-phosphate backbone (an exemplary backbone modification); (vi) modification of the 3′ end or 5′ end of the oligonucleotide, e.g., removal, modification or replacement of a terminal phosphate group or conjugation of a moiety, cap or linker (such 3′ or 5′ cap modifications may comprise a sugar and/or backbone modification); and (vii) modification or replacement of the sugar (an exemplary sugar modification). Certain embodiments comprise a 5′ end modification to an mRNA, gRNA, or nucleic acid. Certain embodiments comprise a 3′ end modification to an mRNA, gRNA, or nucleic acid. A modified RNA can contain 5′ end and 3′ end modifications. A modified RNA can contain one or more modified residues at non-terminal locations. In certain embodiments, a gRNA includes at least one modified residue. In certain embodiments, an mRNA includes at least one modified residue.

Unmodified nucleic acids can be prone to degradation by, e.g., intracellular nucleases or those found in serum. For example, nucleases can hydrolyze nucleic acid phosphodiester bonds. Accordingly, in one aspect the RNAs (e.g. mRNAs, gRNAs) described herein can contain one or more modified nucleosides or nucleotides, e.g., to introduce stability toward intracellular or serum-based nucleases. In some embodiments, the modified gRNA molecules described herein can exhibit a reduced innate immune response when introduced into a population of cells, both in vivo and ex vivo. The term “innate immune response” includes a cellular response to exogenous nucleic acids, including single stranded nucleic acids, which involves the induction of cytokine expression and release, particularly the interferons, and cell death.

Accordingly, in some embodiments, the RNA or nucleic acid in the disclosed LNP compositions comprises at least one modification which confers increased or enhanced stability to the nucleic acid, including, for example, improved resistance to nuclease digestion in vivo. As used herein, the terms “modification” and “modified” as such terms relate to the nucleic acids provided herein, include at least one alteration which preferably enhances stability and renders the RNA or nucleic acid more stable (e.g., resistant to nuclease digestion) than the wild-type or naturally occurring version of the RNA or nucleic acid. As used herein, the terms “stable” and “stability” as such terms relate to the nucleic acids of the present invention, and particularly with respect to the RNA, refer to increased or enhanced resistance to degradation by, for example nucleases (i.e., endonucleases or exonucleases) which are normally capable of degrading such RNA. Increased stability can include, for example, less sensitivity to hydrolysis or other destruction by endogenous enzymes (e.g., endonucleases or exonucleases) or conditions within the target cell or tissue, thereby increasing or enhancing the residence of such RNA in the target cell, tissue, subject and/or cytoplasm. The stabilized RNA molecules provided herein demonstrate longer half-lives relative to their naturally occurring, unmodified counterparts (e.g. the wild-type version of the mRNA). Also contemplated by the terms “modification” and “modified” as such terms related to the mRNA of the LNP compositions disclosed herein are alterations which improve or enhance translation of mRNA nucleic acids, including for example, the inclusion of sequences which function in the initiation of protein translation (e.g., the Kozac consensus sequence). (Kozak, M., Nucleic Acids Res 15 (20): 8125-48 (1987)).

In some embodiments, an RNA or nucleic acid of the LNP compositions disclosed herein has undergone a chemical or biological modification to render it more stable. Exemplary modifications to an RNA include the depletion of a base (e.g., by deletion or by the substitution of one nucleotide for another) or modification of a base, for example, the chemical modification of a base. The phrase “chemical modifications” as used herein, includes modifications which introduce chemistries which differ from those seen in naturally occurring RNA, for example, covalent modifications such as the introduction of modified nucleotides, (e.g., nucleotide analogs, or the inclusion of pendant groups which are not naturally found in such RNA molecules).

In some embodiments of a backbone modification, the phosphate group of a modified residue can be modified by replacing one or more of the oxygens with a different substituent. Further, the modified residue, e.g., modified residue present in a modified nucleic acid, can include the wholesale replacement of an unmodified phosphate moiety with a modified phosphate group as described herein. In some embodiments, the backbone modification of the phosphate backbone can include alterations that result in either an uncharged linker or a charged linker with unsymmetrical charge distribution.

Examples of modified phosphate groups include, phosphorothioate, phosphoroselenates, borano phosphates, borano phosphate esters, hydrogen phosphonates, phosphoroamidates, alkyl or aryl phosphonates and phosphotriesters. The phosphorous atom in an unmodified phosphate group is achiral. However, replacement of one of the non-bridging oxygens with one of the above atoms or groups of atoms can render the phosphorous atom chiral. The stereogenic phosphorous atom can possess either the “R” configuration (herein Rp) or the “S” configuration (herein Sp). The backbone can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either linking oxygen or at both of the linking oxygens. The phosphate group can be replaced by non-phosphorus containing connectors in certain backbone modifications. In some embodiments, the charged phosphate group can be replaced by a neutral moiety. Examples of moieties which can replace the phosphate group can include, without limitation, e.g., methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino.

mRNA

In some embodiments, a composition or formulation disclosed herein comprises an mRNA comprising an open reading frame (ORF) encoding an RNA-guided DNA binding agent, such as a Cas nuclease, or Class 2 Cas nuclease as described herein. In some embodiments, an mRNA comprising an ORF encoding an RNA-guided DNA binding agent, such as a Cas nuclease or Class 2 Cas nuclease, is provided, used, or administered. An mRNA may comprise one or more of a 5′ cap, a 5′ untranslated region (UTR), a 3′ UTRs, and a polyadenine tail. The mRNA may comprise a modified open reading frame, for example to encode a nuclear localization sequence or to use alternate codons to encode the protein.

The mRNA in the disclosed LNP compositions may encode, for example, a secreted hormone, enzyme, receptor, polypeptide, peptide or other protein of interest that is normally secreted. In one embodiment of the invention, the mRNA may optionally have chemical or biological modifications which, for example, improve the stability and/or half-life of such mRNA or which improve or otherwise facilitate protein production.

In addition, suitable modifications include alterations in one or more nucleotides of a codon such that the codon encodes the same amino acid but is more stable than the codon found in the wild-type version of the mRNA. For example, an inverse relationship between the stability of RNA and a higher number cytidines (C's) and/or uridines (U's) residues has been demonstrated, and RNA devoid of C and U residues have been found to be stable to most RNases (Heidenreich, et al. J Biol Chem 269, 2131-8 (1994)). In some embodiments, the number of C and/or U residues in an mRNA sequence is reduced. In another embodiment, the number of C and/or U residues is reduced by substitution of one codon encoding a particular amino acid for another codon encoding the same or a related amino acid. Contemplated modifications to the mRNA nucleic acids of the present invention also include the incorporation of pseudouridines. The incorporation of pseudouridines into the mRNA nucleic acids of the present invention may enhance stability and translational capacity, as well as diminishing immunogenicity in vivo. See, e.g., Karikó, K., et al., Molecular Therapy 16 (11): 1833-1840 (2008). Substitutions and modifications to the mRNA of the present invention may be performed by methods readily known to one or ordinary skill in the art.

The constraints on reducing the number of C and U residues in a sequence will likely be greater within the coding region of an mRNA, compared to an untranslated region, (i.e., it will likely not be possible to eliminate all of the C and U residues present in the message while still retaining the ability of the message to encode the desired amino acid sequence). The degeneracy of the genetic code, however presents an opportunity to allow the number of C and/or U residues that are present in the sequence to be reduced, while maintaining the same coding capacity (i.e., depending on which amino acid is encoded by a codon, several different possibilities for modification of RNA sequences may be possible).

The term modification also includes, for example, the incorporation of non-nucleotide linkages or modified nucleotides into the mRNA sequences of the present invention (e.g., modifications to one or both the 3′ and 5′ ends of an mRNA molecule encoding a functional secreted protein or enzyme). Such modifications include the addition of bases to an mRNA sequence (e.g., the inclusion of a poly A tail or a longer poly A tail), the alteration of the 3′ UTR or the 5′ UTR, complexing the mRNA with an agent (e.g., a protein or a complementary nucleic acid molecule), and inclusion of elements which change the structure of an mRNA molecule (e.g., which form secondary structures).

The poly A tail is thought to stabilize natural messengers. Therefore, in one embodiment a long poly A tail can be added to an mRNA molecule thus rendering the mRNA more stable. Poly A tails can be added using a variety of art-recognized techniques. For example, long poly A tails can be added to synthetic or in vitro transcribed mRNA using poly A polymerase (Yokoe, et al. Nature Biotechnology. 1996; 14: 1252-1256). A transcription vector can also encode long poly A tails. In addition, poly A tails can be added by transcription directly from PCR products. In one embodiment, the length of the poly A tail is at least about 90, 200, 300, 400 at least 500 nucleotides. In one embodiment, the length of the poly A tail is adjusted to control the stability of a modified mRNA molecule of the invention and, thus, the transcription of protein. For example, since the length of the poly A tail can influence the half-life of an mRNA molecule, the length of the poly A tail can be adjusted to modify the level of resistance of the mRNA to nucleases and thereby control the time course of protein expression in a cell. In one embodiment, the stabilized mRNA molecules are sufficiently resistant to in vivo degradation (e.g., by nucleases), such that they may be delivered to the target cell without a transfer vehicle.

In one embodiment, an mRNA can be modified by the incorporation 3′ and/or 5′ untranslated (UTR) sequences which are not naturally found in the wild-type mRNA. In one embodiment, 3′ and/or 5′ flanking sequence which naturally flanks an mRNA and encodes a second, unrelated protein can be incorporated into the nucleotide sequence of an mRNA molecule encoding a therapeutic or functional protein in order to modify it. For example, 3′ or 5′ sequences from mRNA molecules which are stable (e.g., globin, actin, GAPDH, tubulin, histone, or citric acid cycle enzymes) can be incorporated into the 3′ and/or 5′ region of a sense mRNA nucleic acid molecule to increase the stability of the sense mRNA molecule. See, e.g., US2003/0083272.

More detailed descriptions of the mRNA modifications can be found in US2017/0210698A1, at pages 57-68, which content is incorporated herein.

### Template Nucleic Acid

The compositions and methods disclosed herein may include a template nucleic acid. The template may be used to alter or insert a nucleic acid sequence at or near a target site for an RNA-guided DNA binding protein such as a Cas nuclease, e.g., a Class 2 Cas nuclease. In some embodiments, the methods comprise introducing a template to the cell. In some embodiments, a single template may be provided. In other embodiments, two or more templates may be provided such that editing may occur at two or more target sites. For example, different templates may be provided to edit a single gene in a cell, or two different genes in a cell.

In some embodiments, the template may be used in homologous recombination. In some embodiments, the homologous recombination may result in the integration of the template sequence or a portion of the template sequence into the target nucleic acid molecule. In other embodiments, the template may be used in homology-directed repair, which involves DNA strand invasion at the site of the cleavage in the nucleic acid. In some embodiments, the homology-directed repair may result in including the template sequence in the edited target nucleic acid molecule. In yet other embodiments, the template may be used in gene editing mediated by non-homologous end joining. In some embodiments, the template sequence has no similarity to the nucleic acid sequence near the cleavage site. In some embodiments, the template or a portion of the template sequence is incorporated. In some embodiments, the template includes flanking inverted terminal repeat (ITR) sequences.

In some embodiments, the template sequence may correspond to, comprise, or consist of an endogenous sequence of a target cell. It may also or alternatively correspond to, comprise, or consist of an exogenous sequence of a target cell. As used herein, the term “endogenous sequence” refers to a sequence that is native to the cell. The term “exogenous sequence” refers to a sequence that is not native to a cell, or a sequence whose native location in the genome of the cell is in a different location. In some embodiments, the endogenous sequence may be a genomic sequence of the cell. In some embodiments, the endogenous sequence may be a chromosomal or extrachromosomal sequence. In some embodiments, the endogenous sequence may be a plasmid sequence of the cell.

In some embodiments, the template contains ssDNA or dsDNA containing flanking invert-terminal repeat (ITR) sequences. In some embodiments, the template is provided as a vector, plasmid, minicircle, nanocircle, or PCR product.

In some embodiments, the nucleic acid is purified. In some embodiments, the nucleic acid is purified using a precipitation method (e.g., LiCl precipitation, alcohol precipitation, or an equivalent method, e.g., as described herein). In some embodiments, the nucleic acid is purified using a chromatography-based method, such as an HPLC-based method or an equivalent method (e.g, as described herein). In some embodiments, the nucleic acid is purified using both a precipitation method (e.g, LiCl precipitation) and an HPLC-based method. In some embodiments, the nucleic acid is purified by tangential flow filtration (TFF).

The compounds or compositions will generally, but not necessarily, include one or more pharmaceutically acceptable excipients. The term “excipient” includes any ingredient other than the compound(s) of the disclosure, the other lipid component(s) and the biologically active agent. An excipient may impart either a functional (e.g. drug release rate controlling) and/or a non-functional (e.g. processing aid or diluent) characteristic to the compositions. The choice of excipient will to a large extent depend on factors such as the particular mode of administration, the effect of the excipient on solubility and stability, and the nature of the dosage form.

Parenteral formulations are typically aqueous or oily solutions or suspensions. Where the formulation is aqueous, excipients such as sugars (including but not restricted to glucose, mannitol, sorbitol, etc.) salts, carbohydrates and buffering agents (preferably to a pH of from 3 to 9), but, for some applications, they may be more suitably formulated with a sterile non-aqueous solution or as a dried form to be used in conjunction with a suitable vehicle such as sterile, pyrogen-free water (WFI).

While the invention is described in conjunction with the illustrated embodiments, it is understood that they are not intended to limit the invention to those embodiments. On the contrary, the invention is intended to cover all alternatives, modifications, and equivalents, including equivalents of specific features, which may be included within the invention as defined by the appended claims.

Both the foregoing general description and detailed description, as well as the following examples, are exemplary and explanatory only and are not restrictive of the teachings. The section headings used herein are for organizational purposes only and are not to be construed as limiting the desired subject matter in any way. In the event that any literature incorporated by reference contradicts any term defined in this specification, this specification controls. All ranges given in the application encompass the endpoints unless stated otherwise.

### Definitions

It should be noted that, as used in this application, the singular form “a”, “an” and “the” include plural references unless the context clearly dictates otherwise. Thus, for example, reference to “a composition” includes a plurality of compositions and reference to “a cell” includes a plurality of cells and the like. The use of “or” is inclusive and means “and/or” unless stated otherwise.

Unless specifically noted in the above specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; embodiments in the specification that recite “about” various components are also contemplated as “at” the recited components; and embodiments in the specification that recite “consisting essentially of” various components are also contemplated as “consisting of” or “comprising” the recited components (this interchangeability does not apply to the use of these terms in the claims).

Numeric ranges are inclusive of the numbers defining the range. Measured and measureable values are understood to be approximate, taking into account significant digits and the error associated with the measurement. As used in this application, the terms “about” and “approximately” have their art-understood meanings; use of one vs the other does not necessarily imply different scope. Unless otherwise indicated, numerals used in this application, with or without a modifying term such as “about” or “approximately”, should be understood to encompass normal divergence and/or fluctuations as would be appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of a stated reference value unless otherwise stated or otherwise evident from the context (except where such number would exceed 100% of a possible value).

As used herein, the term “contacting” means establishing a physical connection between two or more entities. For example, contacting a mammalian cell with a nanoparticle composition means that the mammalian cell and a nanoparticle are made to share a physical connection. Methods of contacting cells with external entities both in vivo and ex vivo are well known in the biological arts. For example, contacting a nanoparticle composition and a mammalian cell disposed within a mammal may be performed by varied routes of administration (e.g., intravenous, intramuscular, intradermal, and subcutaneous) and may involve varied amounts of nanoparticle compositions. Moreover, more than one mammalian cell may be contacted by a nanoparticle composition.

As used herein, the term “delivering” means providing an entity to a destination. For example, delivering a therapeutic and/or prophylactic to a subject may involve administering a nanoparticle composition including the therapeutic and/or prophylactic to the subject (e.g., by an intravenous, intramuscular, intradermal, or subcutaneous route). Administration of a nanoparticle composition to a mammal or mammalian cell may involve contacting one or more cells with the nanoparticle composition.

As used herein, “encapsulation efficiency” refers to the amount of a therapeutic and/or prophylactic that becomes part of a nanoparticle composition, relative to the initial total amount of therapeutic and/or prophylactic used in the preparation of a nanoparticle composition. For example, if 97 mg of therapeutic and/or prophylactic are encapsulated in a nanoparticle composition out of a total 100 mg of therapeutic and/or prophylactic initially provided to the composition, the encapsulation efficiency may be given as 97%. As used herein, “encapsulation” may refer to complete, substantial, or partial enclosure, confinement, surrounding, or encasement.

As used herein, the term “biodegradable” is used to refer to materials that, when introduced into cells, are broken down by cellular machinery (e.g., enzymatic degradation) or by hydrolysis into components that cells can either reuse or dispose of without significant toxic effect(s) on the cells. In certain embodiments, components generated by breakdown of a biodegradable material do not induce inflammation and/or other adverse effects in vivo. In some embodiments, biodegradable materials are enzymatically broken down. Alternatively or additionally, in some embodiments, biodegradable materials are broken down by hydrolysis.

As used herein, the “N/P ratio” is the molar ratio of ionizable (in the physiological pH range) nitrogen atoms in a lipid to phosphate groups in an RNA, e.g., in a nanoparticle composition including a lipid component and an RNA.

Compositions may also include salts of one or more compounds. Salts may be pharmaceutically acceptable salts. As used herein, “pharmaceutically acceptable salts” refers to derivatives of the disclosed compounds wherein the parent compound is altered by converting an existing acid or base moiety to its salt form (e.g., by reacting a free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecyl sulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety.

As used herein, the “polydispersity index” is a ratio that describes the homogeneity of the particle size distribution of a system. A small value, e.g., less than 0.3, indicates a narrow particle size distribution. In some embodiments, the polydispersity index may be less than 0.1.

As used herein, “transfection” refers to the introduction of a species (e.g., an RNA) into a cell. Transfection may occur, for example, in vitro, ex vivo, or in vivo.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, 5-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched (i.e., linear). The alkyl group can also be substituted or unsubstituted (preferably unsubstituted). For example, the alkyl group can be substituted with one or more groups including, but not limited to, alkyl, cycloalkyl, alkoxy, amino, ether, halide, hydroxy, nitro, silyl, sulfoxo, sulfonate, carboxylate, or thiol, as described herein. A “lower alkyl” group is an alkyl group containing from one to six (e.g., from one to four) carbon atoms.

The term “alkenyl”, as used herein, refers to an aliphatic group containing at least one carbon-carbon double bond and is intended to include both “unsubstituted alkenyls” and “substituted alkenyls”, the latter of which refers to alkenyl moieties having substituents replacing a hydrogen on one or more carbons of the alkenyl group. Such substituents may occur on one or more carbons that are included or not included in one or more double bonds. Moreover, such substituents include all those contemplated for alkyl groups, as discussed below, except where stability is prohibitive. For example, an alkenyl group may be substituted by one or more alkyl, carbocyclyl, aryl, heterocyclyl, or heteroaryl groups is contemplated. Exemplary alkenyl groups include, but are not limited to, vinyl (—CH═CH₂), allyl (—CH₂CH═CH₂), cyclopentenyl (—C₅H₇), and 5-hexenyl (—CH₂CH₂CH₂CH₂CH═CH₂).

An “alkylene” group refers to a divalent alkyl radical, which may be branched or unbranched (i.e., linear). Any of the above mentioned monovalent alkyl groups may be converted to an alkylene by abstraction of a second hydrogen atom from the alkyl. Representative alkylenes include C₂₋₄ alkylene and C₂₋₃ alkylene. Typical alkylene groups include, but are not limited to —CH(CH₃)—, —C(CH₃)₂—, —CH₂CH₂—, —CH₂CH(CH₃)—, —CH₂C(CH₃)₂—, —CH₂CH₂CH₂—, —CH₂CH₂CH₂CH₂—, and the like. The alkylene group can also be substituted or unsubstituted. For example, the alkylene group can be substituted with one or more groups including, but not limited to, alkyl, aryl, heteroaryl, cycloalkyl, alkoxy, amino, ether, halide, hydroxy, nitro, silyl, sulfoxo, sulfonate, sulfonamide, urea, amide, carbamate, ester, carboxylate, or thiol, as described herein.

The term “alkenylene” includes divalent, straight or branched, unsaturated, acyclic hydrocarbyl groups having at least one carbon-carbon double bond and, in one embodiment, no carbon-carbon triple bonds. Any of the above-mentioned monovalent alkenyl groups may be converted to an alkenylene by abstraction of a second hydrogen atom from the alkenyl. Representative alkenylenes include C₂₋₆alkenylenes.

The term “C_{x-y}” when used in conjunction with a chemical moiety, such as alkyl or alkylene, is meant to include groups that contain from x to y carbons in the chain. For example, the term “C_{x-y} alkyl” refers to substituted or unsubstituted saturated hydrocarbon groups, including straight-chain and branched-chain alkyl and alkylene groups that contain from x to y carbons in the chain.

### INCORPORATION BY REFERENCE

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicant reserves the right to physically incorporate into this application any and all materials and information from any such articles, patents, patent applications, or other physical and electronic documents.

### EXAMPLES

TABLE 1
Compounds

| Compound | Structure |
|---|---|
| 1 | 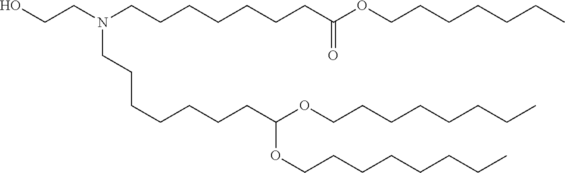 [SMILES: CCCCCCCCOC(CCCCCCCN(CCO)CCCCCCCC(=O)OCCCCCCC)OCCCCCCCC] |
| 2 | 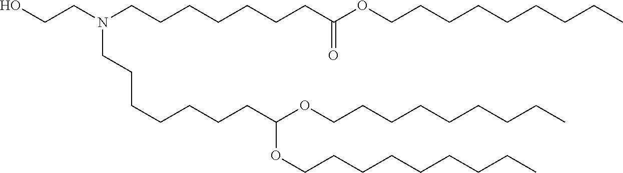 [SMILES: CCCCCCCCCOC(=O)CCCCCCCN(CCO)CCCCCCCC(OCCCCCCCCC)OCCCCCCCCC] |
| 3 | 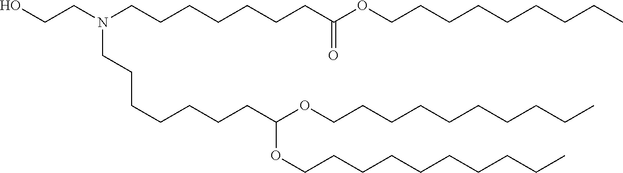 [SMILES: CCCCCCCCCCOC(CCCCCCCN(CCO)CCCCCCCC(=O)OCCCCCCCCC)OCCCCCCCCCC] |
| 4 | 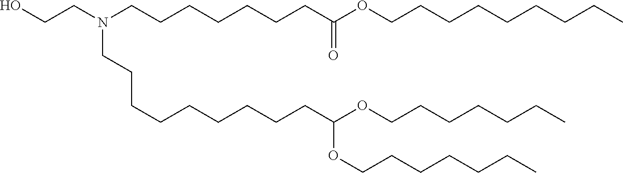 [SMILES: CCCCCCCCCOC(=O)CCCCCCCN(CCO)CCCCCCCCCC(OCCCCCCC)OCCCCCCC] |
| 5 | 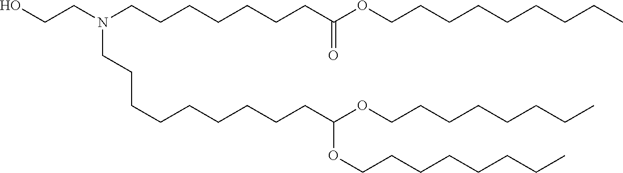 [SMILES: CCCCCCCCCOC(=O)CCCCCCCN(CCO)CCCCCCCCCC(OCCCCCCCC)OCCCCCCCC] |
| 6 | 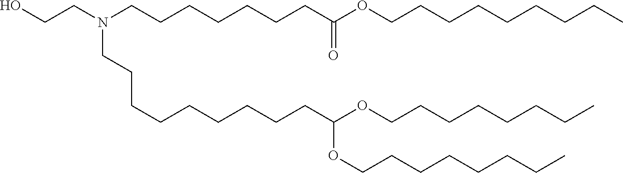 [SMILES: CCCCCCCCCOC(=O)CCCCCCCN(CCO)CCCCCCCCCC(OCCCCCCCC)OCCCCCCCC] |
| 7 | 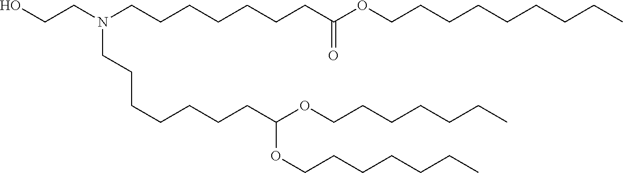 [SMILES: CCCCCCCCCOC(=O)CCCCCCCN(CCO)CCCCCCCC(OCCCCCCC)OCCCCCCC] |
| 8 | 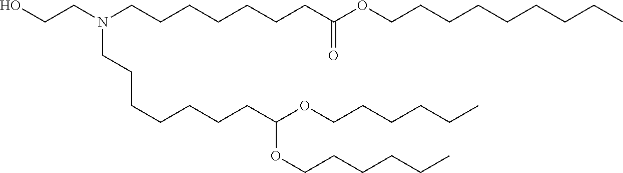 [SMILES: CCCCCCCCCOC(=O)CCCCCCCN(CCO)CCCCCCCC(OCCCCCC)OCCCCCC] |
| 9 | 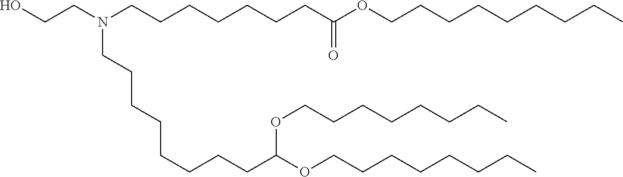 [SMILES: CCCCCCCCCOC(=O)CCCCCCCN(CCO)CCCCCCCCC(OCCCCCCCC)OCCCCCCCC] |
| 10 | 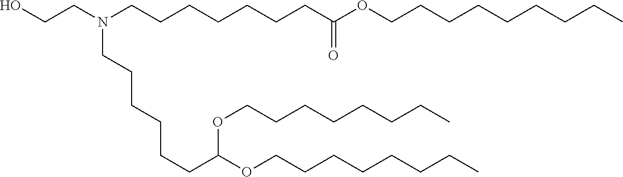 [SMILES: CCCCCCCCCOC(=O)CCCCCCCN(CCO)CCCCCCC(OCCCCCCCC)OCCCCCCCC] |
| 11 | 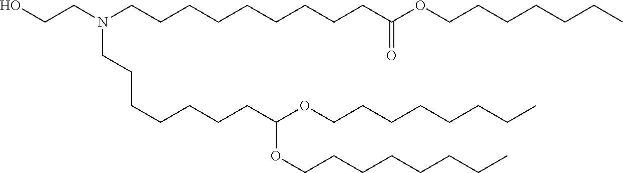 [SMILES: CCCCCCCCOC(CCCCCCCN(CCO)CCCCCCCCCC(=O)OCCCCCCC)OCCCCCCCC] |
| 12 | 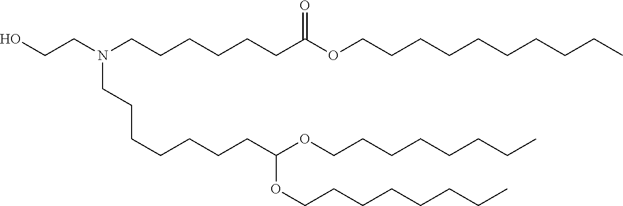 [SMILES: CCCCCCCCCCOC(=O)CCCCCCN(CCO)CCCCCCCC(OCCCCCCCC)OCCCCCCCC] |
| 13 | 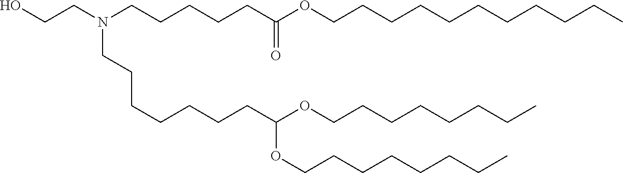 [SMILES: CCCCCCCCCCCOC(=O)CCCCCN(CCO)CCCCCCCC(OCCCCCCCC)OCCCCCCCC] |
| 14 | 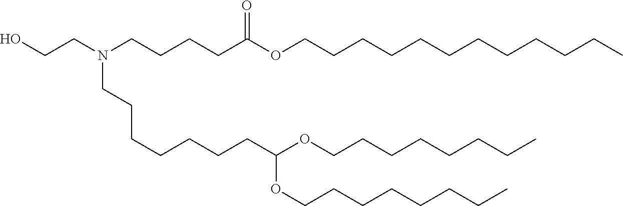 [SMILES: CCCCCCCCCCCCOC(=O)CCCCN(CCO)CCCCCCCC(OCCCCCCCC)OCCCCCCCC] |
| 15 | 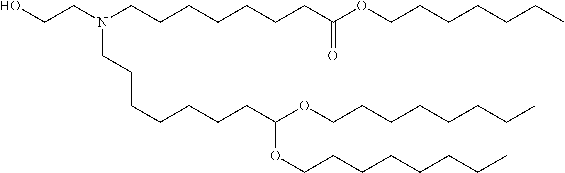 [SMILES: CCCCCCCCOC(CCCCCCCN(CCO)CCCCCCCC(=O)OCCCCCCC)OCCCCCCCC] |
| 16 | 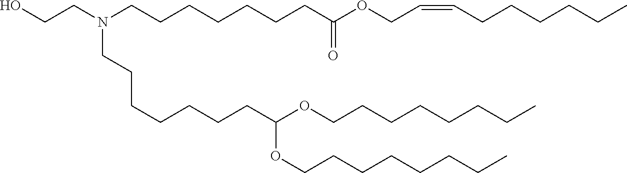 [SMILES: CCCCCC/C=C\COC(=O)CCCCCCCN(CCO)CCCCCCCC(OCCCCCCCC)OCCCCCCCC] |
| 17 | 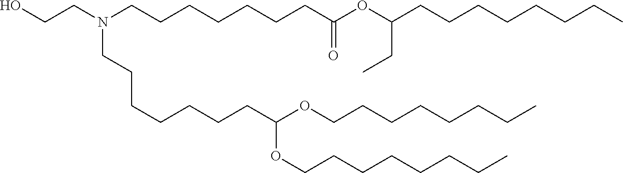 [SMILES: CCCCCCCCOC(CCCCCCCN(CCO)CCCCCCCC(=O)OC(CC)CCCCCCCC)OCCCCCCCC] |
| 18 | 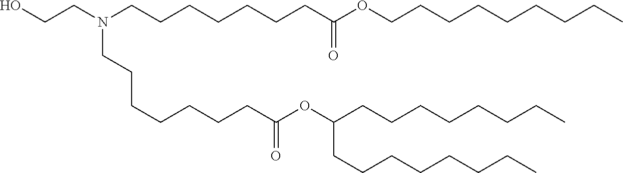 [SMILES: CCCCCCCCCOC(=O)CCCCCCCN(CCO)CCCCCCCC(=O)OC(CCCCCCCC)CCCCCCCC] |
| 19 | 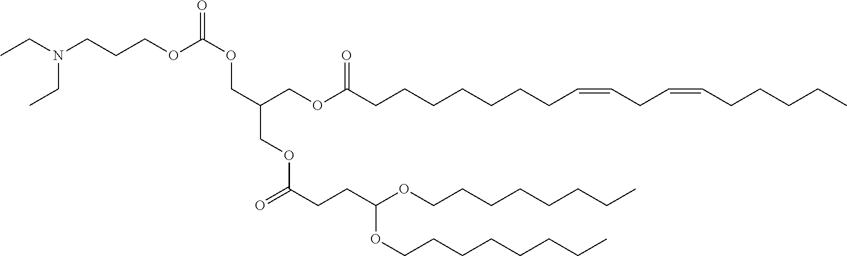 [SMILES: CCCCC/C=C\C/C=C\CCCCCCCC(=O)OCC(COC(=O)CCC(OCCCCCCCC)OCCCCCCCC)COC(=O)OCCCN(CC)CC] |
| 20 | 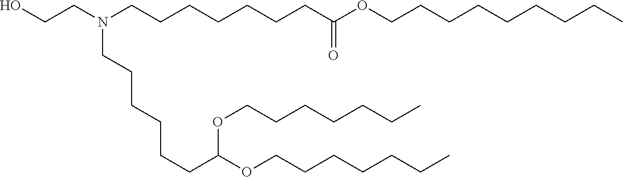 [SMILES: CCCCCCCCCOC(=O)CCCCCCCN(CCO)CCCCCCC(OCCCCCCC)OCCCCCCC] |
| 21 | 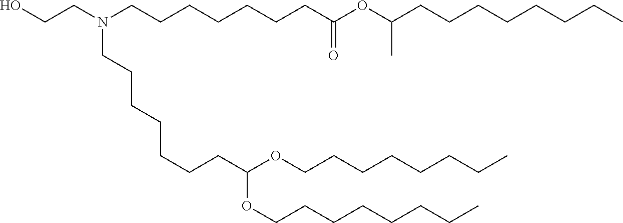 [SMILES: CCCCCCCCOC(CCCCCCCN(CCO)CCCCCCCC(=O)OC(C)CCCCCCCC)OCCCCCCCC] |
| 22 | 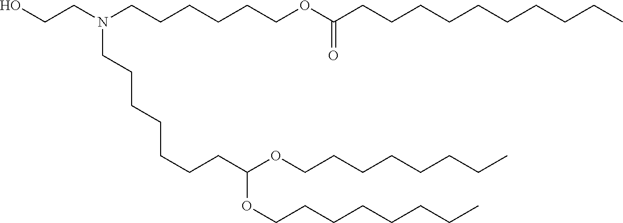 [SMILES: CCCCCCCCCCC(=O)OCCCCCCN(CCO)CCCCCCCC(OCCCCCCCC)OCCCCCCCC] |
| 23 | 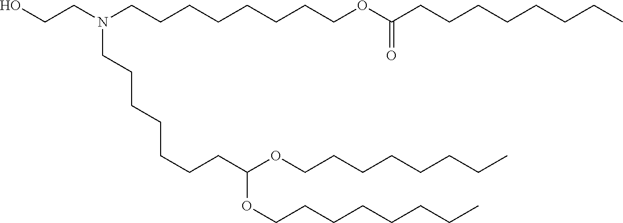 [SMILES: CCCCCCCCOC(CCCCCCCN(CCO)CCCCCCCCOC(=O)CCCCCCCC)OCCCCCCCC] |
| 24 | 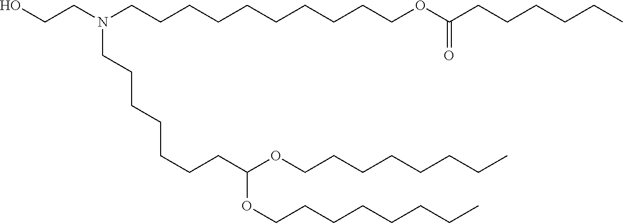 [SMILES: CCCCCCCCOC(CCCCCCCN(CCO)CCCCCCCCCCOC(=O)CCCCCC)OCCCCCCCC] |
| 25 | 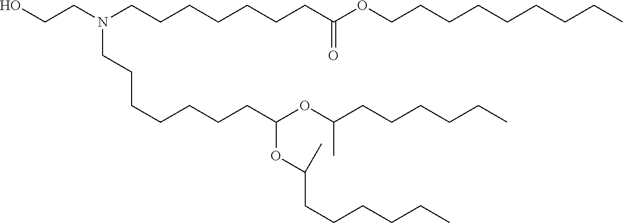 [SMILES: CCCCCCCCCOC(=O)CCCCCCCN(CCO)CCCCCCCC(OC(C)CCCCCC)OC(C)CCCCCC] |
| 26 | 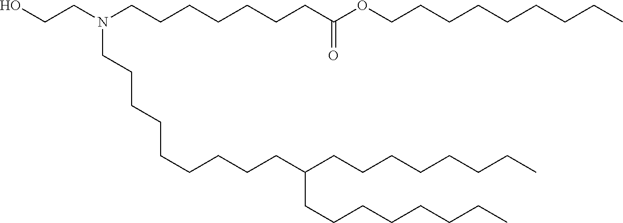 [SMILES: CCCCCCCCCOC(=O)CCCCCCCN(CCO)CCCCCCCCCC(CCCCCCCC)CCCCCCCC] |
| 27 | 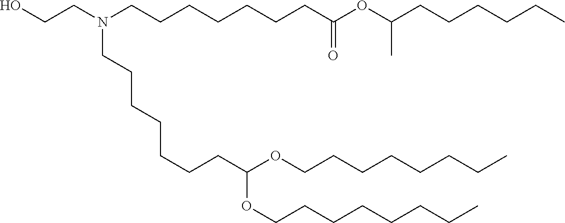 [SMILES: CCCCCCCCOC(CCCCCCCN(CCO)CCCCCCCC(=O)OC(C)CCCCCC)OCCCCCCCC] |
| 28 | 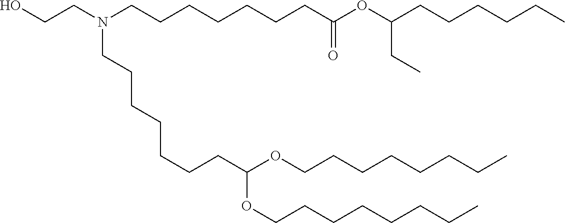 [SMILES: CCCCCCCCOC(CCCCCCCN(CCO)CCCCCCCC(=O)OC(CC)CCCCCC)OCCCCCCCC] |
| 29 | 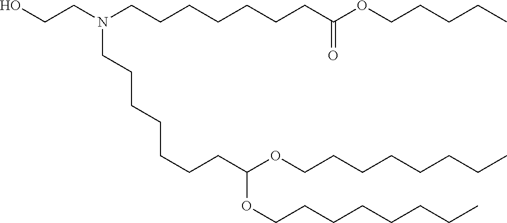 [SMILES: CCCCCCCCOC(CCCCCCCN(CCO)CCCCCCCC(=O)OCCCCC)OCCCCCCCC] |
| 30 | 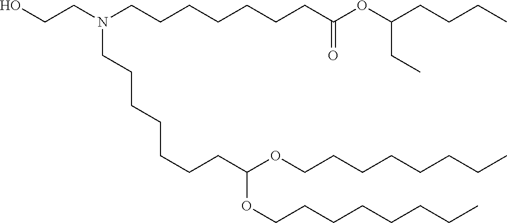 [SMILES: CCCCCCCCOC(CCCCCCCN(CCO)CCCCCCCC(=O)OC(CC)CCCC)OCCCCCCCC] |
| 31 | 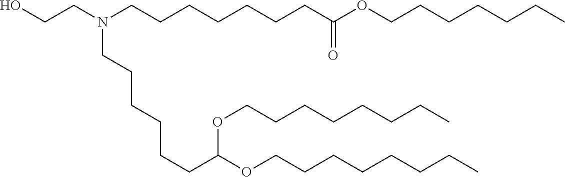 [SMILES: CCCCCCCCOC(CCCCCCN(CCO)CCCCCCCC(=O)OCCCCCCC)OCCCCCCCC] |
| 32 | 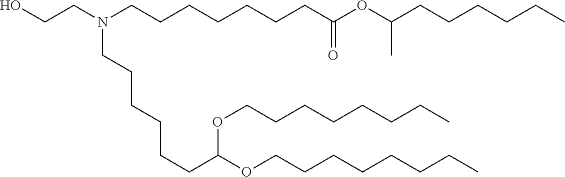 [SMILES: CCCCCCCCOC(CCCCCCN(CCO)CCCCCCCC(=O)OC(C)CCCCCC)OCCCCCCCC] |
| 33 | 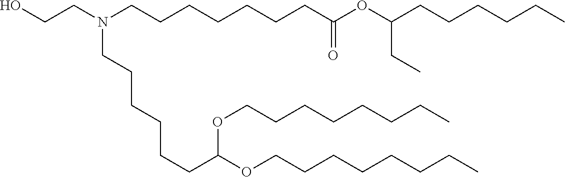 [SMILES: CCCCCCCCOC(CCCCCCN(CCO)CCCCCCCC(=O)OC(CC)CCCCCC)OCCCCCCCC] |
| 34 | 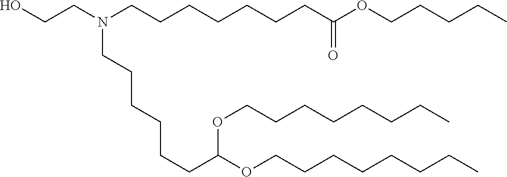 [SMILES: CCCCCCCCOC(CCCCCCN(CCO)CCCCCCCC(=O)OCCCCC)OCCCCCCCC] |
| 35 | 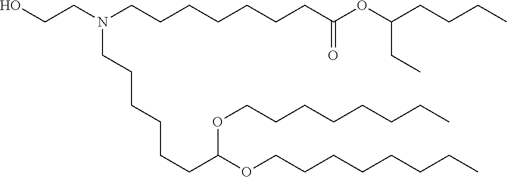 [SMILES: CCCCCCCCOC(CCCCCCN(CCO)CCCCCCCC(=O)OC(CC)CCCC)OCCCCCCCC] |
| 36 | 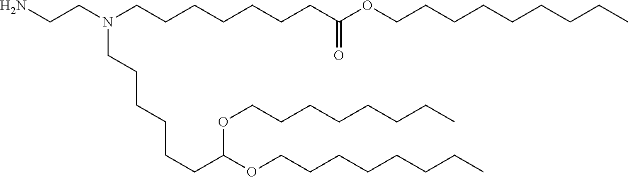 [SMILES: CCCCCCCCCOC(=O)CCCCCCCN(CCN)CCCCCCC(OCCCCCCCC)OCCCCCCCC] |
| 37 | 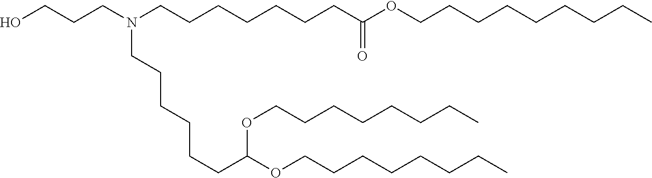 [SMILES: CCCCCCCCCOC(=O)CCCCCCCN(CCCO)CCCCCCC(OCCCCCCCC)OCCCCCCCC] |
| 38 | 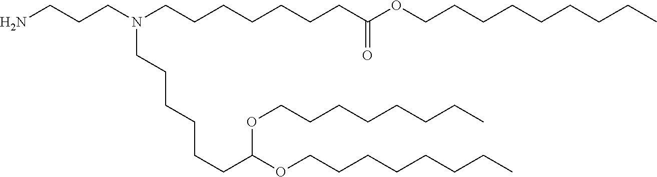 [SMILES: CCCCCCCCCOC(=O)CCCCCCCN(CCCN)CCCCCCC(OCCCCCCCC)OCCCCCCCC] |
| 39 | 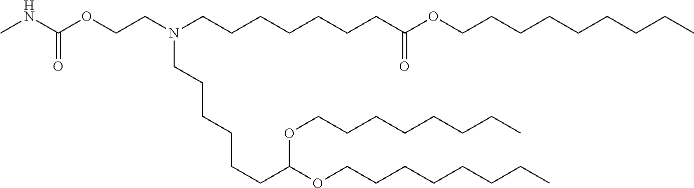 [SMILES: CCCCCCCCCOC(=O)CCCCCCCN(CCCCCCC(OCCCCCCCC)OCCCCCCCC)CCOC(=O)NC] |
| 40 | 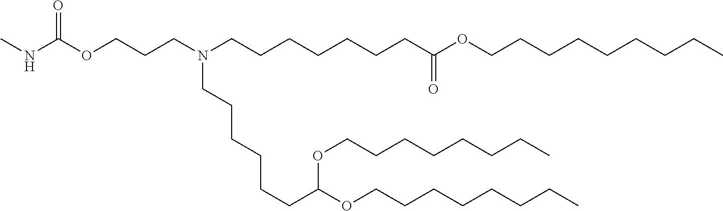 [SMILES: CCCCCCCCCOC(=O)CCCCCCCN(CCCCCCC(OCCCCCCCC)OCCCCCCCC)CCCOC(=O)NC] |
| 41 | 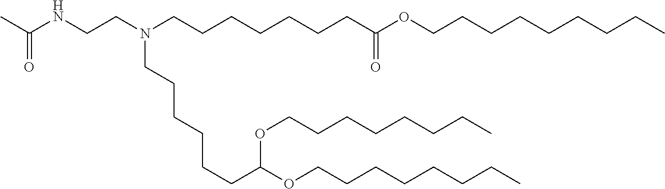 [SMILES: CCCCCCCCCOC(=O)CCCCCCCN(CCCCCCC(OCCCCCCCC)OCCCCCCCC)CCNC(C)=O] |
| 42 | 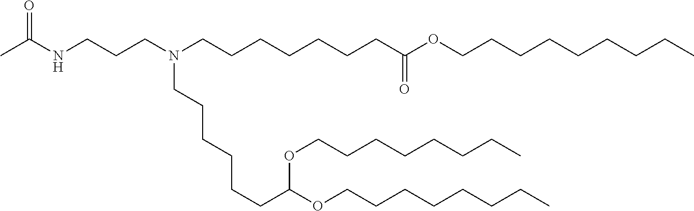 [SMILES: CCCCCCCCCOC(=O)CCCCCCCN(CCCCCCC(OCCCCCCCC)OCCCCCCCC)CCCNC(C)=O] |
| 43 | 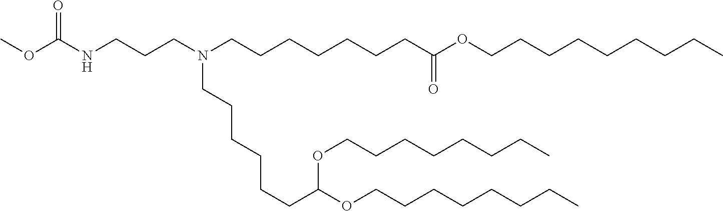 [SMILES: CCCCCCCCCOC(=O)CCCCCCCN(CCCCCCC(OCCCCCCCC)OCCCCCCCC)CCCNC(=O)OC] |
| 44 | 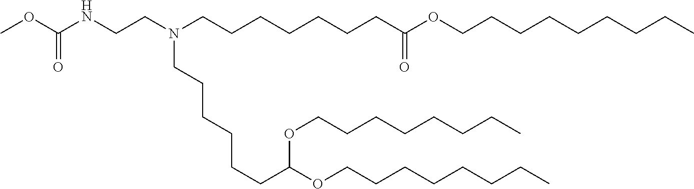 [SMILES: CCCCCCCCCOC(=O)CCCCCCCN(CCCCCCC(OCCCCCCCC)OCCCCCCCC)CCNC(=O)OC] |
| 45 | 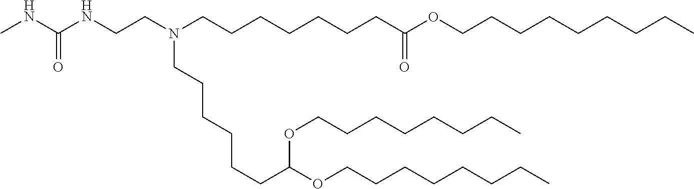 [SMILES: CCCCCCCCCOC(=O)CCCCCCCN(CCCCCCC(OCCCCCCCC)OCCCCCCCC)CCNC(=O)NC] |
| 46 | 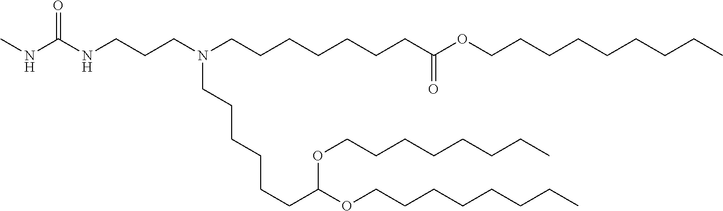 [SMILES: CCCCCCCCCOC(=O)CCCCCCCN(CCCCCCC(OCCCCCCCC)OCCCCCCCC)CCCNC(=O)NC] |
| 47 | 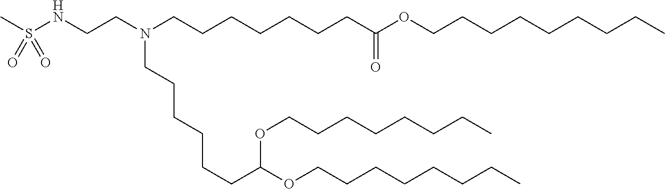 [SMILES: CCCCCCCCCOC(=O)CCCCCCCN(CCCCCCC(OCCCCCCCC)OCCCCCCCC)CCNS(C)(=O)=O] |
| 48 | 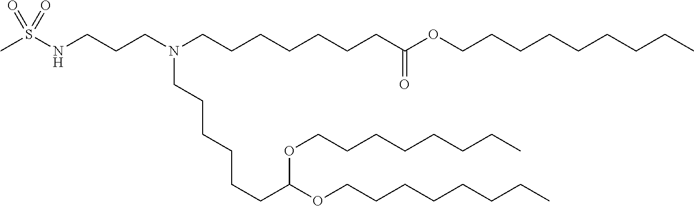 [SMILES: CCCCCCCCCOC(=O)CCCCCCCN(CCCCCCC(OCCCCCCCC)OCCCCCCCC)CCCNS(C)(=O)=O] |
| 49 | 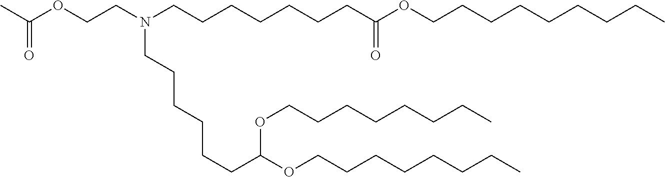 [SMILES: CCCCCCCCCOC(=O)CCCCCCCN(CCCCCCC(OCCCCCCCC)OCCCCCCCC)CCOC(C)=O] |
| 50 | 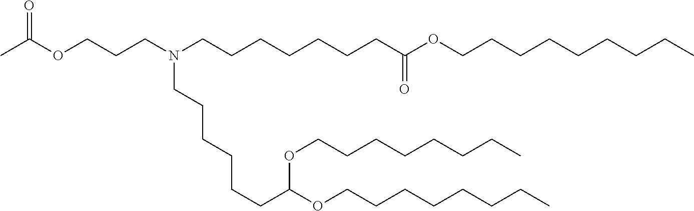 [SMILES: CCCCCCCCCOC(=O)CCCCCCCN(CCCCCCC(OCCCCCCCC)OCCCCCCCC)CCCOC(C)=O] |

### General Information

All reagents and solvents were purchased and used as received from commercial vendors or synthesized according to cited procedures. All intermediates and final compounds were purified using flash column chromatography on silica gel. NMR spectra were recorded on a Bruker or Varian 400 MHz spectrometer, and NMR data were collected in CDCl₃ at ambient temperature. Chemical shifts are reported in parts per million (ppm) relative to CDCl₃ (7.26). Data for ¹H NMR are reported as follows: chemical shift, multiplicity (br=broad, s=singlet, d=doublet, t=triplet, q=quartet, dd=doublet of doublets, dt=doublet of triplets, m=multiplet), coupling constant, and integration. MS data were recorded on a Waters SQD2 mass spectrometer with an electrospray ionization (ESI) source. Purity of the final compounds was determined by UPLC-MS-ELS using a Waters Acquity H-Class liquid chromatography instrument equipped with SQD2 mass spectrometer with photodiode array (PDA) and evaporative light scattering (ELS) detectors.

### Example 1—Compound 1

### Intermediate 1a: nonyl-8-bromooctanoate

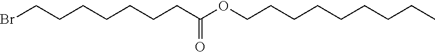 [SMILES: CCCCCCCCCOC(=O)CCCCCCCBr]

To a solution of 8-bromooctanoic acid (5.0 g, 22.4 mmol) and nonan-1-ol (1-2 equiv.) in DCM (56 mL) was added DIEA (2-3 equiv.), DMAP (0.1-0.25 equiv.), and EDC-HCl (1-1.5 equiv.) sequentially at 15-25° C. for at least 4 h. Upon completion, the reaction mixture was diluted with DCM, washed with saturated sodium bicarbonate aqueous solution and brine, dried over sodium sulfate, filtered and concentrated in vacuo. Purification using silica gel chromatography (0-33% EtOAc/hexanes) provided the desired product (4.5 g, 13 mmol, 59% yield) as a clear oil. ¹H NMR (CDCl₃, 400 MHz) δ 4.06 (t, J=6.6 Hz, 2H), 3.40 (t, J=6.8 Hz, 2H), 2.29 (t, J=7.4 Hz, 2H), 1.185 (m, 2H), 1.61 (m, 4H), 1.43 (m, 2H), 1.31 (m, 18H), 0.88 (t, J=6.8 Hz, 3H) ppm.

### Intermediate 1b: nonyl 8-(2-hydroxyethylamino) octanoate

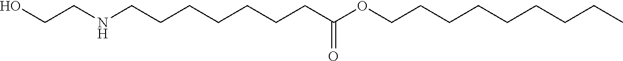 [SMILES: CCCCCCCCCOC(=O)CCCCCCCNCCO]

A solution of Intermediate 1a (12 g, 34.35 mmol) and 2-aminoethanol (20-40 equiv.) in ethanol (EtOH) (10 mL) was stirred for at least 12 h at 20° C. The reaction was then concentrated to remove EtOH, poured into water, and extracted into EtOAc (3×). The combined organic layers were washed 2× with brine, dried with anhydrous sodium sulfate (Na₂SO₄), filtered, and concentrated in vacuo. The crude residue was purified using silica gel chromatography (20-100% EtOAc in petroleum ether, followed by MeOH) to provide the desired product (4 g, 12 mmol, 35% yield) as a yellow solid. ¹H NMR (CDCl₃, 400 MHz) δ 3.99 (t, J=6.8 Hz, 2H), 3.57 (t, J=5.2 Hz, 2H), 2.69 (t, J=5.2 Hz, 2H), 2.54 (t, J=7.2 Hz, 2H), 2.22 (t, J=7.4 Hz, 2H), 1.56-1.20 (m, 24H), 0.81 (t, J=6.8 Hz, 3H) ppm.

### Intermediate 1c: 8-bromooctanal

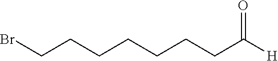

To a solution of 8-bromooctan-1-ol (45.1 mL, 263 mmol) in DCM (700 mL) was added pyridinium chlorochromate (PCC) (1-2 equiv.). After stirring at 15° C. for at least 2 h, the reaction mixture was filtered and concentrated in vacuo. The crude residue was purified using silica gel chromatography (2-20% EtOAc in petroleum ether) to provide the desired product (37.5 g, 163.0 mmol, 62% yield) as a colorless oil. ¹H NMR (CDCl₃, 400 MHz) δ 9.77 (t, J=1.8 Hz, 1H), 3.40 (t, J=6.8 Hz, 2H), 2.43 (m, 2H), 1.86 (m, 2H), 1.63 (m, 2H), 1.45 (m, 2H) 1.34 (m, 4H) ppm.

### Intermediate 1d: 8-bromo-1,1-dioctoxy-octane

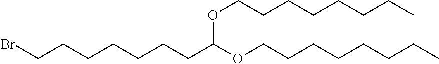 [SMILES: CCCCCCCCOC(CCCCCCCBr)OCCCCCCCC]

To a solution of 8-bromooctanal (12.5 g, 60.3 mmol) and octan-1-ol (2-3 equiv.) in DCM (300 mL) was added p-toluenesulfonic acid monohydrate (0.1-0.2 equiv.) and Na₂SO₄ (2-3 equiv.). The reaction mixture was stirred at 15° C. for at least 24 h, then filtered, and concentrated in vacuo. The crude residue was purified using silica gel chromatography (100% petroleum ether) to provide the desired product (6 g, 13.4 mmol, 22% yield) as a colorless oil. ¹H NMR (CDCl₃, 400 MHz) δ 4.46 (t, J=5.6 Hz, 1H), 3.56 (m, 2H), 3.41 (m, 4H), 1.84 (m, 2H), 1.59 (m, 6H), 1.33-1.28 (m, 34H), 0.89 (t, J=6.6 Hz, 6H) ppm.

### Compound 1: nonyl 8-((8,8-bis(octyloxy)octyl)(2-hydroxyethyl)amino)octanoate

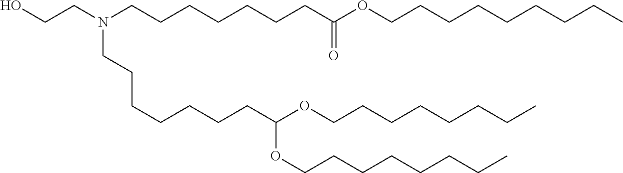 [SMILES: CCCCCCCCCOC(=O)CCCCCCCN(CCO)CCCCCCCC(OCCCCCCCC)OCCCCCCCC]

A mixture of Intermediate 1d (1 g, 2.22 mmol), Intermediate 1b (0.9-1.1 equiv.), K₂CO₃ (2-4 equiv.) and KI (0.1-0.5 equiv.) in 3:1 MeCN/CPME (0.1-0.5 M) was degassed and purged with N2 three times. The reaction mixture was warmed to 82° C. and stirred for at least 2 h under inert atmosphere. The reaction mixture was then diluted with water and extracted at least 2× into EtOAc. The combined organic layers were washed with brine, dried over Na₂SO₄, filtered, and concentrated in vacuo. The crude residue was purified using silica gel chromatography (10-33% EtOAc in petroleum ether) to provide the desired product (700 mg, 1.00 mmol, 45% yield) as a colorless oil. ¹H NMR (CDCl₃, 400 MHz) δ 4.50 (t, J=5.8 Hz, 1H), 4.05 (t, J=6.8 Hz, 2H), 3.54 (m, 4H), 3.40 (m, 2H), 2.56 (t, J=5.4 Hz, 2H), 2.42 (t, J=7.4 Hz, 4H), 2.29 (t, J=7.6 Hz, 2H), 1.58 (m, 10H), 1.45-1.21 (m, 50H) 0.88 (t, J=6.8 Hz, 9H) ppm. MS: 699.29 m/z [M+H].

### Example 2—Compound 2

### Intermediate 2a: 1-(8-bromo-1-nonoxy-octoxy)nonane

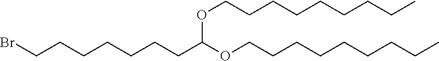 [SMILES: CCCCCCCCCOC(CCCCCCCBr)OCCCCCCCCC]

Intermediate 2a was synthesized in 24% yield from Intermediate 1c and nonan-1-ol using the method employed for Intermediate 1d. ¹H NMR (CDCl₃, 400 MHz) δ 4.46 (t, J=5.8 Hz, 1H), 3.56 (m, 2H), 3.41 (m, 4H), 1.86 (m, 2H), 1.57 (m, 6H), 1.33 (m, 32H), 0.89 (6, J=6.8 Hz, 6H) ppm.

### Compound 2: 8-[8,8-di(nonoxy)octyl-(2-hydroxyethyl)amino]octanoate

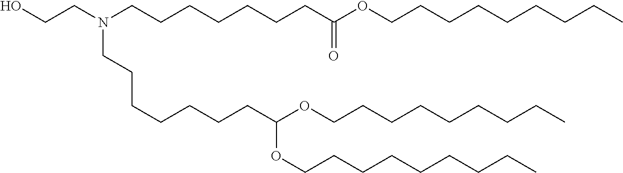 [SMILES: CCCCCCCCCOC(=O)CCCCCCCN(CCO)CCCCCCCC(OCCCCCCCCC)OCCCCCCCCC]

Compound 2 was synthesized 54% yield from Intermediate 1b and Intermediate 2a using the method employed for Compound 1. H NMR (CDCl₃, 400 MHz) δ 4.50 (t, J=5.6 Hz, 1H), 4.05 (t, J=6.8 Hz, 2H), 3.54 (m, 4H), 3.40 (m, 2H), 2.56 (t, J=5.4 Hz, 2H), 2.42 (t, J=7.4 Hz, 4H), 2.29 (t, J=7.6 Hz, 2H), 1.58 (m, 10H), 1.46-1.21 (m, 54H), 0.88 (t, J=6.6 Hz, 9H) ppm. MS: 727.01 m/z [M+H].

### Example 3—Compound 3

### Intermediate 3a: 1-(8-bromo-1-decoxy-octoxy)decane

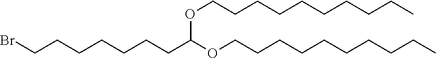 [SMILES: CCCCCCCCCCOC(CCCCCCCBr)OCCCCCCCCCC]

Intermediate 3a was synthesized in 24% yield from Intermediate 1c and decan-1-ol using the method employed for Intermediate 1d. ¹H NMR (CDCl₃, 400 MHz) δ 4.46 (t, J=5.8 Hz, 1H), 3.56 (m, 2H), 3.40 (m, 4H), 1.86 (m, 2H), 1.57 (m, 6H), 1.33 (m, 36H), 0.89 (t, J=6.8 Hz, 6H) ppm.

### Compound 3: nonyl 8-[8,8-didecoxyoctyl(2-hydroxyethyl)amino]octanoate

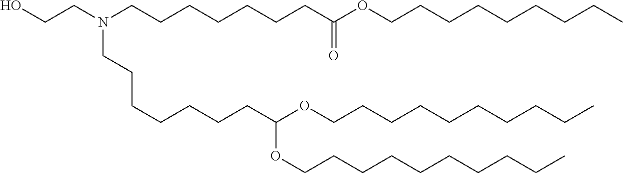 [SMILES: CCCCCCCCCCOC(CCCCCCCN(CCO)CCCCCCCC(=O)OCCCCCCCCC)OCCCCCCCCCC]

Compound 3 was synthesized in 28% yield from Intermediate 1b and Intermediate 3a using the method employed for Compound 1. H NMR (CDCl₃, 400 MHz) δ 4.50 (t, J=5.8 Hz, 1H), 4.05 (t, J=6.8 Hz, 2H), 3.53 (m, 4H), 3.39 (m, 2H), 2.56 (t, J=5.4 Hz, 2H), 2.43 (t, J=7.4 Hz, 4H), 2.29 (t, J=7.6 Hz, 2H), 1.58 (m, 10H), 1.46-1.20 (m, 58H), 0.88 (t, J=6.6 Hz, 9H) ppm. MS: 755.04 m/z [M+H].

### Example 4—Compound 4

### Intermediate 4a: 10-bromooctanal

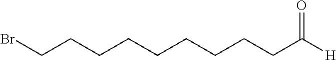 [SMILES: O=CCCCCCCCCCBr]

Intermediate 4a was synthesized in 55% yield from 10-bromooctanol using the method employed for Intermediate 1c. ¹H NMR (CDCl₃, 400 MHz) δ 9.77 (s, 1H), 3.41 (t, J=7.0 Hz, 2H), 2.42 (t, J=7.4 Hz, 2H), 1.85 (m, 2H), 1.63 (m, 2H), 1.42 (m, 2H), 1.30 (m, 8H) ppm.

### Intermediate 4b: 10-bromo-1, 1-diheptoxy-decane

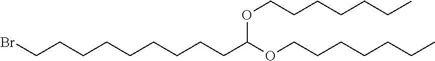 [SMILES: CCCCCCCOC(CCCCCCCCCBr)OCCCCCCC]

Intermediate 4b was synthesized in 32% yield from Intermediate 4a and heptan-1-ol using the method employed for Intermediate 1d. ¹H NMR (CDCl₃, 400 MHz) δ 4.46 (t, J=5.8 Hz, 1H), 3.56 (m, 2H), 3.41 (m, 4H), 1.86 (m, 2H), 1.58 (m, 6H), 1.33 (m, 28H), 0.89 (t, J=7.0 Hz, 6H) ppm.

### Compound 4: nonyl 8-[10, 10-diheptoxydecyl(2-hydroxyethyl)amino]octanoate

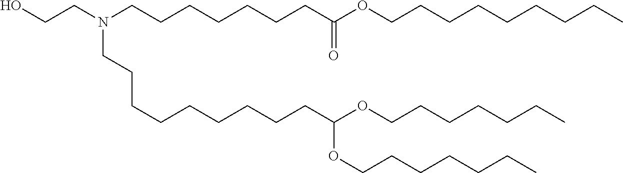 [SMILES: CCCCCCCCCOC(=O)CCCCCCCN(CCO)CCCCCCCCCC(OCCCCCCC)OCCCCCCC]

Compound 4 was synthesized in 19% yield from Intermediate 1b and Intermediate 4b using the method employed for Compound 1. ¹H NMR (CDCl₃, 400 MHz) δ 4.46 (t, J=5.8 Hz, 1H), 4.05 (t, J=6.6 Hz, 2H), 3.55 (m, 4H), 3.40 (m, 4H), 2.59 (t, J=5.4 Hz, 2H), 2.45 (m, 4H), 2.29 (t, J=7.4 Hz, 2H), 1.59 (m, 10H), 1.44-1.22 (m, 50H), 0.88 (t, J=7.0 Hz, 9H) ppm. MS: 699.53 m/z [M+H].

### Example 5—Compound 5

### Intermediate 5a: 10-bromo-1, 1-diheptoxy-decane

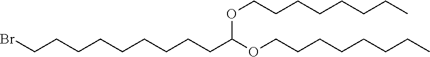 [SMILES: CCCCCCCCOC(CCCCCCCCCBr)OCCCCCCCC]

Intermediate 5a was synthesized in 34% yield from Intermediate 4a and octan-1-ol using the method employed for Intermediate 1d. ¹H NMR (CDCl₃, 400 MHz) δ 4.45 (t, J=5.8 Hz, 1H), 3.55 (m, 2H), 3.40 (m, 4H), 1.85 (m, 2H), 1.57 (m, 6H), 1.33 (m, 32H), 0.88 (t, J=6.8 Hz, 6H) ppm.

### Compound 5: 8-[10, 10-dioctoxydecyl (2-hydroxyethyl) amino] octanoate

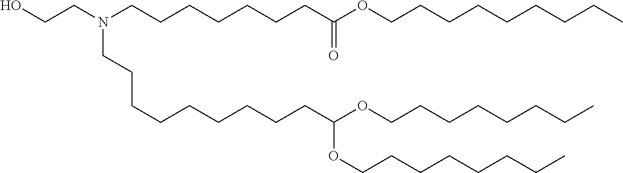 [SMILES: CCCCCCCCCOC(=O)CCCCCCCN(CCO)CCCCCCCCCC(OCCCCCCCC)OCCCCCCCC]

Compound 5 was synthesized in 27% yield from Intermediate 1b and Intermediate 5a using the method employed for Compound 1. ¹H NMR (CDCl₃, 400 MHz) δ 4.46 (t, J=5.8 Hz, 1H), 4.06 (t, J=6.8 Hz, 2H), 3.56 (m, 4H), 3.40 (m, 2H), 2.58 (t, J=5.4 Hz, 2H), 2.45 (m, 4H), 2.29 (t, J=7.6 Hz, 2H), 1.59 (m, 10H), 1.47-1.25 (m, 54H), 0.89 (t, J=6.6 Hz, 9H) ppm. UPLC-MS-ELS: r.t.=6.58 min, 727.54 m/z [M+H].

### Example 6—Compound 6

### Intermediate 6a: 10-bromo-1,1-bis(nonyloxy)decane

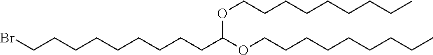 [SMILES: CCCCCCCCCOC(CCCCCCCCCBr)OCCCCCCCCC]

Intermediate 6a was synthesized in 41% yield from Intermediate 4a and nonan-1-ol using the method employed for Intermediate 1d. ¹H NMR (CDCl₃, 400 MHz) δ 4.46 (t, J=5.8 Hz, 1H), 3.56 (m, 2H), 3.41 (m, 4H), 1.86 (m, 2H), 1.58 (m, 6H), 1.42-1.28 (m, 36H), 0.89 (t, J=6.8 Hz, 6H) ppm.

### Compound 6: 8-[10,10-di(nonoxy)decyl-(2-hydroxyethyl)amino]octanoate

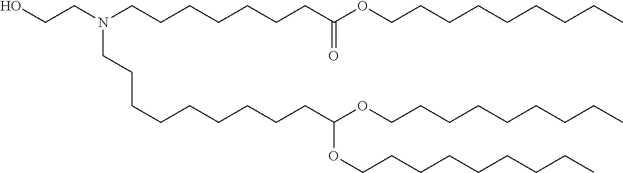 [SMILES: CCCCCCCCCOC(=O)CCCCCCCN(CCO)CCCCCCCCCC(OCCCCCCCCC)OCCCCCCCCC]

Compound 6 was synthesized in 41% yield from Intermediate 1b and Intermediate 6a using the method employed for Compound 1. ¹H NMR (CDCl₃, 400 MHz) δ 4.45 (t, J=5.8 Hz, 1H), 4.05 (t, J=6.8 Hz, 2H), 3.54 (m, 4H), 3.39 (m, 2H), 2.57 (t, J=5.4 Hz, 2H), 2.44 (t, J=7.6 Hz, 4H), 2.29 (t, J=7.6 Hz, 2H), 1.58 (m, 10H), 1.46-1.24 (m, 58H), 0.88 (t, J=6.6 Hz, 9H) ppm. MS: 755.71 m/z [M+H].

### Example 7—Compound 7

### Intermediate 7a: 8-bromo-1,1-bis(heptyloxy)octane

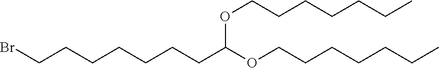 [SMILES: CCCCCCCOC(CCCCCCCBr)OCCCCCCC]

Intermediate 7a was synthesized in 39% yield from Intermediate 1c and heptan-1-ol using the method employed for Intermediate 1d. ¹H NMR (CDCl₃, 400 MHz) δ 4.46 (t, J=5.8 Hz, 1H), 3.56 (m, 2H), 3.41 (m, 4H), 1.86 (m, 2H), 1.57 (m, 6H), 1.32 (m, 24H), 0.89 (t, J=7.0 Hz, 6H) ppm.

### Compound 7: nonyl 8-((8,8-bis(heptyloxy)octyl)(2-hydroxyethyl)amino)octanoate

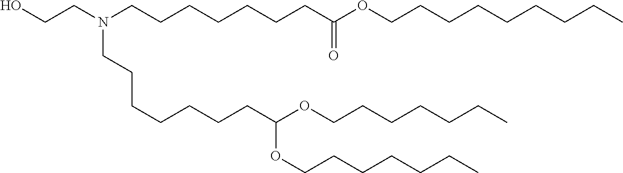 [SMILES: CCCCCCCCCOC(=O)CCCCCCCN(CCO)CCCCCCCC(OCCCCCCC)OCCCCCCC]

Compound 7 was synthesized in 22% yield from Intermediate 1b and Intermediate 7a using the method employed for Compound 1. ¹H NMR (CDCl₃, 400 MHz) δ 4.45 (t, J=5.8 Hz, 1H), 4.05 (t, J=6.8 Hz, 2H), 3.60-3.48 (m, 4H), 3.40 (m, 2H), 2.56 (t, J=5.4 Hz, 2H), 2.43 (dd, J=8.5, 6.3 Hz, 4H), 2.29 (t, J=7.6 Hz, 2H), 1.67-1.52 (m, 10H), 1.48-1.19 (m, 46H), 0.88 (m, 9H) ppm. MS: 671.66 m/z [M+H].

### Example 8—Compound 8

### Intermediate 8a: 8-bromo-1,1-bis(hexyloxy)octane

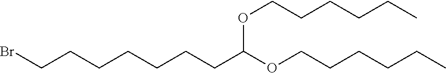 [SMILES: CCCCCCOC(CCCCCCCBr)OCCCCCC]

Intermediate 8a was synthesized in 38% yield from Intermediate 1c and hexan-1-ol using the method employed for Intermediate 1d. ¹H NMR (CDCl₃, 400 MHz) δ 4.46 (t, J=5.6 Hz, 1H), 3.57 (m, 2H), 3.40 (m, 4H), 1.85 (m, 2H), 1.57 (m, 6H), 1.35 (m, 20H), 0.89 (t, J=6.8 Hz, 6H) ppm.

### Compound 8: nonyl 8-((8,8-bis(hexyloxy)octyl)(2-hydroxyethyl)amino)octanoate

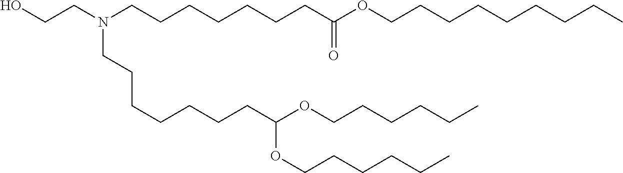 [SMILES: CCCCCCCCCOC(=O)CCCCCCCN(CCO)CCCCCCCC(OCCCCCC)OCCCCCC]

Compound 8 was synthesized in 13% yield from Intermediate 1b and Intermediate 8a using the method employed for Compound 1. ¹H NMR (CDCl₃, 400 MHz) δ 4.45 (t, J=5.8 Hz, 1H), 4.05 (t, J=6.8 Hz, 2H), 3.60-3.49 (m, 4H), 3.40 (m, 2H), 2.57 (t, J=5.4 Hz, 2H), 2.43 (t, J=7.6 Hz, 4H), 2.29 (t, J=7.6 Hz, 2H), 1.58 (m, 10H), 1.47-1.19 (m, 42H), 0.88 (m, 9H) ppm. MS: 643.58 m/z [M+H].

### Example 9—Compound 9

### Intermediate 9a: 9-bromononanal

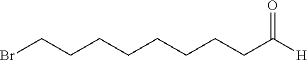 [SMILES: O=CCCCCCCCCBr]

Intermediate 9a was synthesized in 40% yield from 9-bromooctanol using the method employed for Intermediate 1c. ¹H NMR (CDCl₃, 400 MHz) δ 9.70 (t, J=1.8 Hz, 1H), 3.34 (t, J=6.8 Hz, 2H), 2.36 (m, 2H), 1.78 (m, 2H), 1.57 (m, 2H), 1.36 (m, 2H), 1.26 (m, 6H) ppm.

### Intermediate 9b: 9-bromo-1,1-bis(octyloxy)nonane

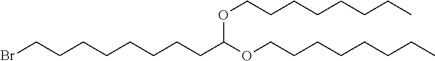 [SMILES: CCCCCCCCOC(CCCCCCCCBr)OCCCCCCCC]

Intermediate 9b was synthesized in 44% yield from Intermediate 9a and octan-1-ol using the method employed for Intermediate 1d. ¹H NMR (CDCl₃, 400 MHz) δ 4.46 (t, J=5.6 Hz, 1H), 3.57 (m, 2H), 3.41 (m, 4H), 1.86 (m, 2H), 1.57 (m, 6H), 1.31 (m, 30H), 0.89 (t, J=6.8 Hz, 6H) ppm.

### Compound 9: nonyl 8-((9,9-bis(octyloxy)nonyl)(2-hydroxyethyl)amino)octanoate

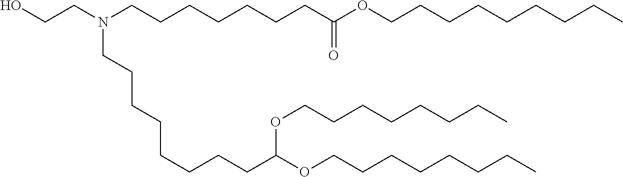 [SMILES: CCCCCCCCCOC(=O)CCCCCCCN(CCO)CCCCCCCCC(OCCCCCCCC)OCCCCCCCC]

Compound 9 was synthesized in 17% yield from Intermediate 1b and Intermediate 9b using the method employed for Compound 1. ¹H NMR (CDCl₃, 400 MHz) δ 4.45 (t, J=5.8 Hz, 1H), 4.05 (t, J=6.8 Hz, 2H), 3.62-3.49 (m, 4H), 3.40 (m, 2H), 2.57 (t, J=5.4 Hz, 2H), 2.44 (t, J=7.6 Hz, 4H), 2.29 (t, J=7.6 Hz, 2H), 1.58 (m, 10H), 1.48-1.19 (m, 52H), 0.88 (t, J=6.6 Hz, 9H) ppm. MS: 713.52 m/z [M+H].

### Example 10—Compound 10

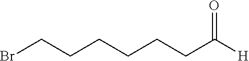

Intermediate 10a was synthesized in 35% yield from 7-bromoheptanol using the method employed for Intermediate 1c. ¹H NMR (CDCl₃, 400 MHz) δ 9.77 (s, 1H), 3.41 (t, J=6.6 Hz, 2H), 2.44 (m, 2H), 1.87 (m, 2H), 1.65 (m, 2H), 1.47 (m, 2H), 1.37 (m, 2H) ppm.

### Intermediate 10b: 1-((7-bromo-1-(octyloxy)heptyl)oxy)octane

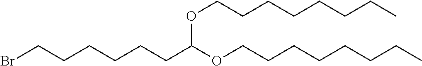 [SMILES: CCCCCCCCOC(CCCCCCBr)OCCCCCCCC]

Intermediate 10b was synthesized in 42% yield from Intermediate 10a and octan-1-ol using the method employed for Intermediate 1d. H NMR (CDCl₃, 400 MHz) δ 4.46 (t, J=5.6 Hz, 1H), 3.57 (m, 2H), 3.41 (m, 4H), 1.85 (m, 2H), 1.58 (m, 6H), 1.33 (m, 26H), 0.89 (t, J=6.8 Hz, 6H) ppm.

### Compound 10: nonyl 8-((7,7-bis(octyloxy)heptyl)(2-hydroxyethyl)amino)octanoate

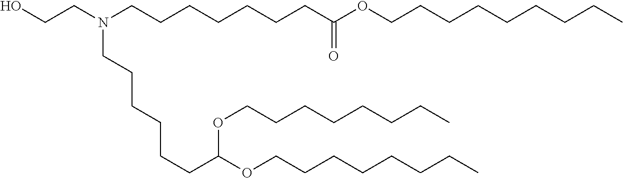 [SMILES: CCCCCCCCCOC(=O)CCCCCCCN(CCO)CCCCCCC(OCCCCCCCC)OCCCCCCCC]

Compound 10 was synthesized in 19% yield from Intermediate 1b and Intermediate 10b using the method employed for Compound 1. ¹H NMR (CDCl₃, 400 MHz) δ 4.46 (t, J=5.8 Hz, 1H), 4.05 (t, J=6.8 Hz, 2H), 3.59-3.49 (m, 4H), 3.39 (m, 2H), 2.56 (t, J=5.4 Hz, 2H), 2.43 (t, J=7.4 Hz, 4H), 2.29 (t, J=7.6 Hz, 2H), 1.58 (m, 10H), 1.47-1.21 (m, 48H), 0.88 (t, J=6.6 Hz, 9H) ppm. MS: 685.75 m/z [M+H].

### Example 11—Compound 11

### Intermediate 11a: 2-((8,8-bis(octyloxy)octyl)amino)ethan-1-ol

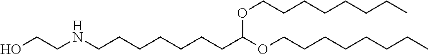 [SMILES: CCCCCCCCOC(CCCCCCCNCCO)OCCCCCCCC]

To a solution of Intermediate 1d (24 g, 115.88 mmol) and octan-1-ol (2-4 equiv.) in DCM (240 mL) was added TsOH.H₂O (0.1-0.3 equiv.) and Na₂SO₄ (2-3 equiv.). The mixture was stirred at 25° C. for at least 12 h. Upon completion, the reaction mixture was concentrated under reduced pressure to remove DCM. The residue was diluted with water and extracted with 3× with EtOAc. The combined organic layers were washed with brine, dried over Na₂SO₄, filtered and concentrated under reduced pressure to give a residue. The residue was purified by column chromatography (EtOAc/hexanes) to afford product as a colorless oil (25 g, 48%). ¹H NMR (400 MHz, CDCl₃) δ 4.38 (t, J=5.8 Hz, 1H), 3.61-3.54 (m, 2H), 3.49 (dt, J=9.3, 6.6 Hz, 2H), 3.33 (dt, J=9.3, 6.7 Hz, 2H), 2.75-2.66 (m, 2H), 2.55 (t, J=7.2 Hz, 2H), 1.97 (d, J=12.5 Hz, 3H), 1.58-1.35 (m, 8H), 1.34-1.01 (m, 27H), 0.93-0.72 (m, 6H) ppm. MS: 430.4 m/z [M+H].

### Intermediate 11b: heptyl 10-bromodecanoate

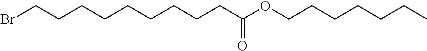 [SMILES: CCCCCCCOC(=O)CCCCCCCCCBr]

Intermediate 11b was synthesized in 32% yield from 10-bromodecanoic acid and heptan-1-01 using the method employed for Intermediate 1a. ¹H NMR (400 MHz, CDCl₃) δ 3.99 (t, J=6.7 Hz, 2H), 3.33 (t, J=6.9 Hz, 2H), 2.22 (t, J=7.5 Hz, 2H), 1.83-1.68 (m, 2H), 1.55 (d, J=14.3 Hz, 4H), 1.35 (t, J=7.5 Hz, 2H), 1.22 (m, 16H), 0.86-0.78 (m, 3H) ppm.

### Compound 11: heptyl 10-((8,8-bis(octyloxy)octyl)(2-hydroxyethyl)amino)decanoate

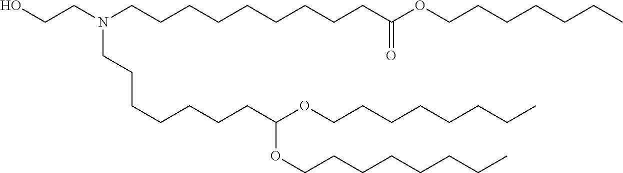 [SMILES: CCCCCCCCOC(CCCCCCCN(CCO)CCCCCCCCCC(=O)OCCCCCCC)OCCCCCCCC]

Compound 11 was synthesized in 19% yield from Intermediate 11a and Intermediate 11b using the method employed for Compound 11. ¹H NMR (400 MHz, CDCl₃) δ 4.45 (t, J=5.7 Hz, 1H), 4.05 (t, J=6.8 Hz, 2H), 3.67-3.60 (m, 2H), 3.55 (dt, J=9.3, 6.7 Hz, 2H), 3.40 (dt, J=9.3, 6.7 Hz, 2H), 2.70 (s, 2H), 2.58 (s, 4H), 2.29 (t, J=7.5 Hz, 2H), 1.67-1.44 (m, 15H), 1.29 (m, 46H), 0.94-0.81 (m, 9H) ppm. MS: 699.35 m/z [M+H].

### Example 12—Compound 12

### Intermediate 12a: decyl 7-bromoheptanoate

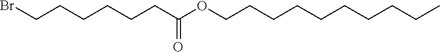 [SMILES: CCCCCCCCCCOC(=O)CCCCCCBr]

Intermediate 12a was synthesized in 26% yield from 7-bromoheptanoic acid and decan-1-ol using the method employed for Intermediate 1a. H NMR (400 MHz, CDCl₃) δ 4.09 (t, J=6.7 Hz, 2H), 3.44 (t, J=6.8 Hz, 2H), 2.34 (t, J=7.5 Hz, 2H), 1.96-1.83 (m, 2H), 1.70-1.57 (m, 4H), 1.49 (m, 2H), 1.44-1.22 (m, 16H), 0.97-0.85 (m, 3H) ppm.

### Compound 12: decyl 7-((8,8-bis(octyloxy)octyl)(2-hydroxyethyl)amino)heptanoate

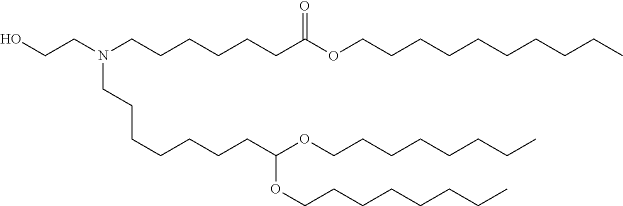 [SMILES: CCCCCCCCCCOC(=O)CCCCCCN(CCO)CCCCCCCC(OCCCCCCCC)OCCCCCCCC]

Compound 12 was synthesized in 56% yield from Intermediate 11a and Intermediate 12a using the method employed for Compound 11. ¹H NMR (400 MHz, CDCl₃) δ 4.45 (t, J=5.7 Hz, 1H), 4.05 (t, J=6.8 Hz, 2H), 3.60-3.51 (m, 4H), 3.40 (dt, J=9.3, 6.7 Hz, 2H), 2.61 (t, J=5.3 Hz, 2H), 2.55-2.41 (m, 4H), 2.29 (t, J=7.5 Hz, 2H), 1.69-1.51 (m, 10H), 1.51-1.20 (m, 49H), 0.94-0.83 (m, 9H) ppm. MS: 699.52 m/z [M+H].

### Example 13—Compound 13

### Intermediate 13a: undecyl 6-bromohexanoate

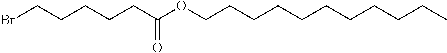 [SMILES: CCCCCCCCCCCOC(=O)CCCCCBr]

Intermediate 13a was synthesized in 22% yield from 6-bromohexanoic acid and undecan-1-01 using the method employed for Intermediate 1a. ¹H NMR (400 MHz, CDCl₃) δ 4.06 (t, J=6.7 Hz, 2H), 3.40 (t, J=6.8 Hz, 2H), 2.31 (t, J=7.4 Hz, 2H), 1.87 (dt, J=14.2, 6.9 Hz, 2H), 1.70-1.57 (m, 4H), 1.53-1.42 (m, 2H), 1.38-1.19 (m, 16H), 0.87 (t, J=6.7 Hz, 3H) ppm.

### Compound 13: undecyl 6-((8,8-bis(octyloxy)octyl)(2-hydroxyethyl)amino)hexanoate

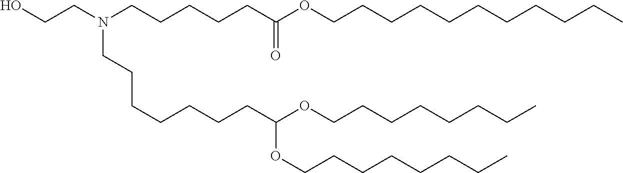 [SMILES: CCCCCCCCCCCOC(=O)CCCCCN(CCO)CCCCCCCC(OCCCCCCCC)OCCCCCCCC]

Compound 13 was synthesized in 64% yield from Intermediate 11a and Intermediate 13a using the method employed for Compound 11. ¹H NMR (400 MHz, CDCl₃) δ 4.45 (t, J=5.7 Hz, 1H), 4.05 (t, J=6.8 Hz, 2H), 3.60-3.52 (m, 4H), 3.40 (dt, J=9.3, 6.7 Hz, 2H), 2.62 (t, J=5.3 Hz, 2H), 2.50 (q, J=6.7 Hz, 4H), 2.30 (t, J=7.5 Hz, 2H), 1.70-1.40 (m, 15H), 1.40-1.17 (m, 45H), 0.93-0.83 (m, 9H) ppm. MS: 699.31 m/z [M+H].

### Example 14—Compound 14

### Intermediate 14a: dodecyl 5-bromopentanoate

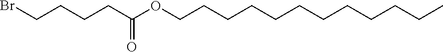 [SMILES: CCCCCCCCCCCCOC(=O)CCCCBr]

Intermediate 14a was synthesized in 21% yield from 5-bromopentanoic and dodecan-1-ol using the method employed in Intermediate 1a. ¹H NMR (400 MHz, CDCl₃) δ 4.00 (t, J=6.7 Hz, 2H), 3.35 (t, J=6.6 Hz, 2H), 2.27 (t, J=7.3 Hz, 2H), 1.83 (m, 2H), 1.71 (m, 2H), 1.55 (t, J=7.1 Hz, 2H), 1.31-1.13 (m, 18H), 0.85-0.78 (m, 3H) ppm.

### Compound 14: dodecyl 5-((8,8-bis(octyloxy)octyl)(2-hydroxyethyl)amino)pentanoate

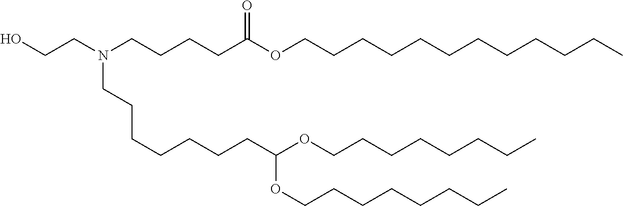 [SMILES: CCCCCCCCCCCCOC(=O)CCCCN(CCO)CCCCCCCC(OCCCCCCCC)OCCCCCCCC]

Compound 14 was synthesized in 62% yield from Intermediate 11a and Intermediate 14a using the method employed for Compound 11. ¹H NMR (400 MHz, CDCl₃) δ 4.45 (t, J=5.7 Hz, 1H), 4.06 (t, J=6.8 Hz, 2H), 3.63-3.49 (m, 4H), 3.40 (dt, J=9.3, 6.7 Hz, 2H), 2.62 (t, J=5.3 Hz, 2H), 2.58-2.44 (m, 4H), 2.32 (t, J=7.3 Hz, 2H), 1.68-1.40 (m, 15H), 1.40-1.19 (m, 47H), 0.94-0.83 (m, 9H) ppm. MS: 699.48 m/z [M+H].

### Example 15—Compound 15

### Intermediate 15a: heptyl 8-bromooctanoate

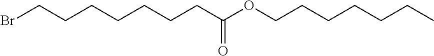 [SMILES: CCCCCCCOC(=O)CCCCCCCBr]

Intermediate 15a was synthesized in 15% yield from 8-bromooctanoic acid and heptan-1-ol using the method employed for Intermediate 1a. ¹H NMR (400 MHz, CDCl₃) δ 3.99 (t, J=6.7 Hz, 2H), 3.33 (t, J=6.8 Hz, 2H), 2.23 (t, J=7.5 Hz, 2H), 1.78 (m, 2H), 1.63-1.50 (m, 4H), 1.42-1.13 (m, 14H), 0.87-0.77 (m, 3H) ppm.

### Compound 15: heptyl 8-((8,8-bis(octyloxy)octyl)(2-hydroxyethyl)amino)octanoate

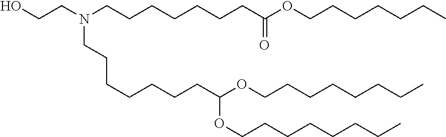 [SMILES: CCCCCCCCOC(CCCCCCCN(CCO)CCCCCCCC(=O)OCCCCCCC)OCCCCCCCC]

Compound 15 was synthesized in 64% yield from Intermediate 11a and Intermediate 15a using the method=employed for Compound 11. ¹H NMR (400 MHz, CDCl₃) δ 4.45 (t, J=5.7 Hz, 1H), 4.05 (t, J=6.7 Hz, 2H), 3.62-3.50 (m, 4H), 3.40 (dt, J=9.3, 6.7 Hz, 2H), 2.64 (t, J=5.2 Hz, 2H), 2.51 (t, J=7.6 Hz, 4H), 2.29 (t, J=7.5 Hz, 2H), 1.66-1.40 (m, 15H), 1.40-1.19 (m, 43H), 0.88 (m, 9H) ppm. MS: 671.84 m/z [M+H].

### Example 16—Compound 16

### Intermediate 16a: (Z)-non-2-en-1-yl 8-bromooctanoate

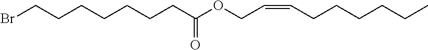 [SMILES: CCCCCC/C=C\COC(=O)CCCCCCCBr]

Intermediate 16a was synthesized in 26% yield from 8-bromooctanoic acid and (Z)-non-2-en-1-ol using the method employed for Intermediate 1a. ¹H NMR (400 MHz, CDCl₃) δ 5.70-5.58 (m, 1H), 5.58-5.47 (m, 1H), 4.62 (dd, J=6.9, 1.3 Hz, 2H), 3.40 (t, J=6.8 Hz, 2H), 2.30 (t, J=7.5 Hz, 2H), 2.09 (m, 2H), 1.85 (m, 2H), 1.67-1.58 (m, 2H), 1.52-1.09 (m, 13H), 0.94-0.80 (m, 3H) ppm.

### Compound 16: (Z)-non-2-en-1-yl 8-((8,8-bis(octyloxy)octyl)(2-hydroxyethyl)amino)octanoate

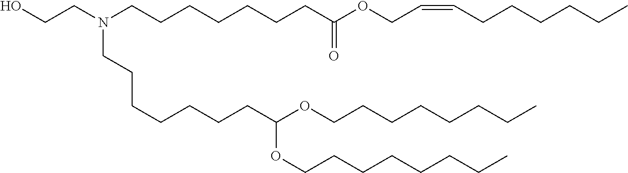 [SMILES: CCCCCC/C=C\COC(=O)CCCCCCCN(CCO)CCCCCCCC(OCCCCCCCC)OCCCCCCCC]

Compound 16 was synthesized in 59% yield from Intermediate 11a and Intermediate 16a using the method employed for Compound 11. ¹H NMR (400 MHz, CDCl₃) δ 5.70-5.58 (m, 1H), 5.52 (m, 1H), 4.62 (dd, J=6.9, 1.3 Hz, 2H), 4.45 (t, J=5.7 Hz, 1H), 3.64-3.48 (m, 4H), 3.40 (dt, J=9.3, 6.7 Hz, 2H), 2.64 (t, J=5.3 Hz, 2H), 2.51 (t, J=7.6 Hz, 4H), 2.30 (t, J=7.5 Hz, 2H), 2.09 (m, 2H), 1.68-1.41 (m, 12H), 1.41-1.18 (m, 41H), 0.96-0.81 (m, 9H) ppm. MS: 697.33 m/z [M+H]. MS: 697.33 m/z [M+H].

### Example 17—Compound 17

### Intermediate 17a: undecan-3-yl 8-bromooctanoate

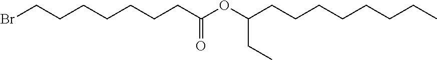 [SMILES: CCCCCCCCC(CC)OC(=O)CCCCCCCBr]

Intermediate 17a was synthesized in 50% yield from 8-bromooctanoic acid and undecan-3-ol using the method employed for Intermediate 1a. ¹H NMR (400 MHz, CDCl₃) δ 4.74 (m, 1H), 3.33 (t, J=6.8 Hz, 2H), 2.22 (t, J=7.5 Hz, 2H), 1.85-1.67 (m, 2H), 1.62-1.09 (m, 25H), 0.89-0.74 (m, 6H) ppm.

### Compound 17: undecan-3-yl 8-((8,8-bis(octyloxy)octyl)(2-hydroxyethyl)amino)octanoate

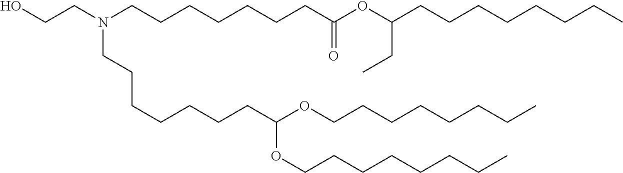 [SMILES: CCCCCCCCOC(CCCCCCCN(CCO)CCCCCCCC(=O)OC(CC)CCCCCCCC)OCCCCCCCC]

Compound 17 was synthesized in 65% yield from Intermediate 11a and Intermediate 17a using the method employed for Compound 11. ¹H NMR (400 MHz, CDCl₃) δ 4.80 (m, 1H), 4.45 (t, J=5.8 Hz, 1H), 3.55 (dt, J=9.3, 6.4 Hz, 4H), 3.40 (dt, J=9.3, 6.7 Hz, 2H), 2.62 (t, J=5.3 Hz, 2H), 2.49 (t, J=7.6 Hz, 4H), 2.28 (t, J=7.5 Hz, 2H), 1.66-1.40 (m, 16H), 1.40-1.17 (m, 45H), 0.87 (m, 12H) ppm. MS: 727.34 m/z [M+H].

### Example 18—Compound 18

### Compound 18: heptadecan-9-yl 8-((2-hydroxyethyl)(8-(nonyloxy)-8-oxooctyl)amino)octanoate

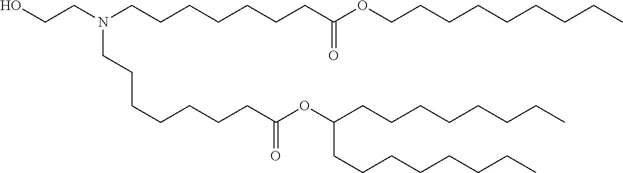 [SMILES: CCCCCCCCCOC(=O)CCCCCCCN(CCO)CCCCCCCC(=O)OC(CCCCCCCC)CCCCCCCC]

Compound 18 was synthesized according to methods described in Mol. Ther. 2018, 26, 1509-1519 (Compound 5) and US 2017/0210698 A1 (Compound 18). ¹H NMR (400 MHz, CDCl₃) δ ¹H NMR (400 MHz, CDCl₃) δ 4.86 (m, 1H), 4.05 (t, J=6.7 Hz, 2H), 3.59 (br t, J=5.1 Hz, 2H), 2.75-2.39 (br m, 6H), 2.28 (m, 4H), 1.61 (m, 6H), 1.49 (m, 8H), 1.38-1.20 (m, 49H), 0.87 (m, 9H) ppm; MS: 711 m/z [M+H].

### Example 19—Compound 19

### Compound 19: 3-((4,4-bis(octyloxy)butanoyl)oxy)-2-(((((3-(diethylamino)propoxy))-carbonyl)oxy)methyl)propyl (9Z,12Z)-octadeca-9,12-dienoate

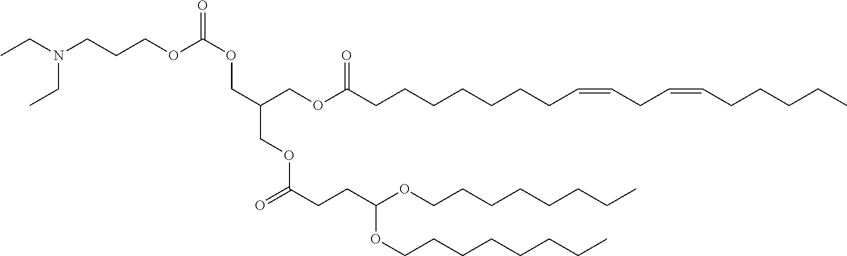 [SMILES: CCCCC/C=C\C/C=C\CCCCCCCC(=O)OCC(COC(=O)CCC(OCCCCCCCC)OCCCCCCCC)COC(=O)OCCCN(CC)CC]

Compound 19 was synthesized according to methods described in WO 2015/095340 A1 (Example 13). ¹H NMR (CDCl₃, 400 MHz) δ 5.35 (m, 4H), 4.48 (t, J=5.6 Hz, 1H), 4.17 (m, 8H), 3.56 (m, 2H), 3.40 (m, 2H), 2.77 (t, J=6.6 Hz, 2H), 2.55 (q, J=7.2 Hz, 6H), 2.40 (m, 3H), 2.30 (t, J=7.6 Hz, 2H), 2.05 (q, J=6.8 Hz, 4H), 1.92 (m, 2H), 1.84 (m, 2H), 1.57 (m, 6H), 1.30 (m, 34H), 1.03 (t, J=7.2 Hz, 6H), 0.88 (m, 9H) ppm; MS: 853 m/z [M+H].

### Example 20—Compound 20

### Intermediate 20a: 7-bromo-1,1-bis(heptyloxy)heptane

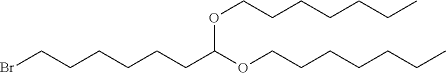 [SMILES: CCCCCCCOC(CCCCCCBr)OCCCCCCC]

Intermediate 20a was synthesized in 24% yield from Intermediate 10a and heptan-1-ol using the method employed for Intermediate 1d. ¹H NMR (400 MHz, CDCl₃) δ 9.77 (t, J=1.7 Hz, 1H), 4.05 (t, J=6.7 Hz, 1H), 3.40 (t, J=6.8 Hz, 4H), 2.44 (td, J=7.3, 1.7 Hz, 2H), 2.33 (dt, J=25.0, 7.4 Hz, 1H), 1.93-1.79 (m, 4H), 1.71-1.53 (m, 5H), 1.51-1.29 (m, 9H).

### Compound 20: nonyl 8-((7,7-bis(heptyloxy)heptyl)(2-hydroxyethyl)amino)octanoate

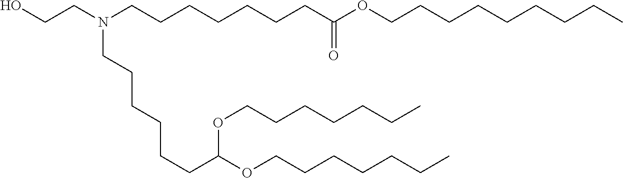 [SMILES: CCCCCCCCCOC(=O)CCCCCCCN(CCO)CCCCCCC(OCCCCCCC)OCCCCCCC]

Compound 20 was synthesized in 60% yield from Intermediate 1b and Intermediate 20a using the method employed for Compound 1. H NMR (400 MHz, CDCl₃) 4.45 (t, J=5.7 Hz, 1H), 4.05 (t, J=6.7 Hz, 2H), 3.55 (dt, J=9.3, 5.9 Hz, 4H), 3.40 (dt, J=9.3, 6.7 Hz, 2H), 2.62 (t, J=5.3 Hz, 2H), 2.49 (t, J=7.6 Hz, 4H), 2.29 (t, J=7.5 Hz, 2H), 1.67-1.41 (m, 15H), 1.41-1.19 (m, 40H), 0.96-0.81 (m, 9H). MS: 657.2 m/z [M+H].

### Example 21—Compound 21

### Intermediate 21a: decan-2-yl 8-bromooctanoate

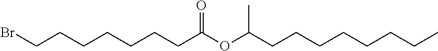 [SMILES: CCCCCCCCC(C)OC(=O)CCCCCCCBr]

To a solution containing 8-bromooctanoic acid (2.0 g, 1.0 equiv) in DCM (0.4 M) was added decan-2-ol (1.0 equiv), DMAP (0.2 equiv), Et₃N (3.5 equiv), and EDCI (1.2 equiv). The reaction was stirred at room temperature for 168 h. Upon completion, the reaction was quenched by the addition of water and DCM. The organic layer was washed 1× with 1 M HCl and 1× with 5% NaHCO₃. The organic layer was dried over Na₂SO₄, filtered, and concentrated. Purification by column (EtOAc/hex) afforded product as a colorless oil (485 mg, 12%). ¹H NMR (400 MHz, CDCl₃) δ 4.97-4.82 (m, 1H), 3.53 (t, J=6.7 Hz, 2H), 2.27 (t, J=7.5 Hz, 2H), 1.76 (dq, J=7.8, 6.8 Hz, 2H), 1.66-1.58 (m, 2H), 1.51-1.40 (m, 3H), 1.37-1.23 (m, 15H), 1.19 (d, J=6.3 Hz, 3H), 0.93-0.84 (m, 3H).

### Compound 21: decan-2-yl 8-((8,8-bis(octyloxy)octyl)(2-hydroxyethyl)amino)octanoate

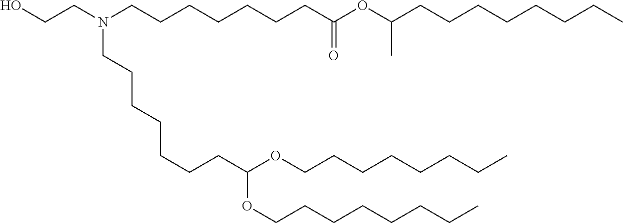 [SMILES: CCCCCCCCOC(CCCCCCCN(CCO)CCCCCCCC(=O)OC(C)CCCCCCCC)OCCCCCCCC]

Compound 21 was synthesized in 29% yield from Intermediate 11a and Intermediate 21a using the method employed for Compound 11. ¹H NMR (400 MHz, CDCl₃) δ 4.89 (ddt, J=12.1, 7.4, 6.3 Hz, 1H), 4.45 (t, J=5.7 Hz, 1H), 3.62-3.50 (m, 4H), 3.40 (dt, J=9.3, 6.7 Hz, 2H), 2.64 (t, J=5.2 Hz, 2H), 2.51 (t, J=7.6 Hz, 4H), 2.26 (t, J=7.5 Hz, 2H), 1.66-1.40 (m, 15H), 1.29 (dd, J=16.9, 6.2 Hz, 44H), 1.19 (d, J=6.2 Hz, 3H), 0.95-0.82 (m, 9H). MS: 713.5 m/z [M+H].

### Example 22—Compound 22

### Intermediate 22a: 6-bromohexyl undecanoate

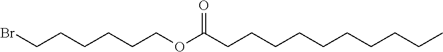 [SMILES: CCCCCCCCCCC(=O)OCCCCCCBr]

A mixture of undecanoic acid (5 g, 1.0 equiv), 6-bromohexan-1-ol (1.0 equiv), EDCI (1.0 equiv), DMAP (0.16 equiv) and DIPEA (3.0 equiv) in DCM (0.2 M) was degassed and purged with N2 for 3 times, and then the mixture was stirred at 20° C. for 5 h under inert atmosphere. Upon completion, the reaction mixture was concentrated under reduced pressure to remove DCM. The residue was diluted with H₂O and extracted 3× with EtOAc. The combined organic layers were dried over Na₂SO₄, filtered, and concentrated. Purification by column (EtOAc/hexanes) afforded product as a colorless oil (2.3 g, 25%). ¹H NMR (400 MHz, CDCl₃) δ 4.00 (t, J=6.6 Hz, 2H), 3.34 (t, J=6.8 Hz, 2H), 2.22 (t, J=7.6 Hz, 2H), 1.84-1.74 (m, 2H), 1.63-1.50 (m, 4H), 1.45-1.36 (m, 2H), 1.36-1.28 (m, 2H), 1.20 (d, J=9.9 Hz, 15H), 0.86-0.78 (m, 3H).

### Compound 22: 6-((8,8-bis(octyloxy)octyl)(2-hydroxyethyl)amino)hexyl undecanoate

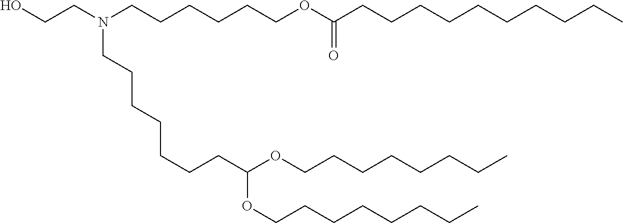 [SMILES: CCCCCCCCCCC(=O)OCCCCCCN(CCO)CCCCCCCC(OCCCCCCCC)OCCCCCCCC]

Compound 22 was synthesized in 63% yield from Intermediate 11a and Intermediate 22a using the method employed for Compound 11. ¹H NMR (400 MHz, CDCl₃) δ 4.45 (t, J=5.7 Hz, 1H), 4.05 (t, J=6.7 Hz, 2H), 3.64 (t, J=5.2 Hz, 2H), 3.55 (dt, J=9.3, 6.7 Hz, 2H), 3.40 (dt, J=9.3, 6.7 Hz, 2H), 2.71 (t, J=5.2 Hz, 2H), 2.60 (t, J=7.6 Hz, 4H), 2.29 (t, J=7.6 Hz, 2H), 1.69-1.05 (m, 62H), 0.95-0.79 (m, 9H). MS: 699.4 m/z [M+H].

### Example 23—Compound 23

### Intermediate 23a: 8-bromooctyl nonanoate

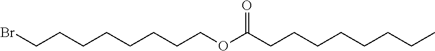 [SMILES: CCCCCCCCC(=O)OCCCCCCCCBr]

Intermediate 23a was synthesized in 19% yield from nonanoic acid and 8-bromooctan-1-ol using the method employed in Intermediate 22a. ¹H NMR (400 MHz, CDCl₃) δ 3.99 (t, J=6.7 Hz, 2H), 3.34 (t, J=6.8 Hz, 2H), 2.22 (t, J=7.6 Hz, 2H), 1.78 (p, J=6.9 Hz, 2H), 1.55 (t, J=7.0 Hz, 4H), 1.42-1.10 (m, 19H), 0.87-0.74 (m, 3H).

### Compound 23: 8-((8,8-bis(octyloxy)octyl)(2-hydroxyethyl)amino)octyl nonanoate

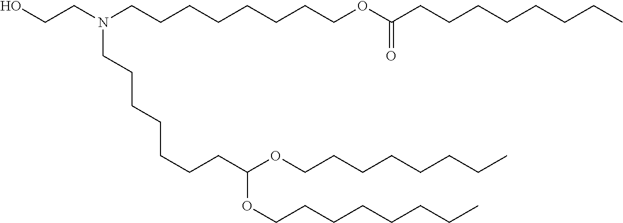 [SMILES: CCCCCCCCOC(CCCCCCCN(CCO)CCCCCCCCOC(=O)CCCCCCCC)OCCCCCCCC]

Compound 23 was synthesized in 32% yield from Intermediate 11a and Intermediate 23a using the method employed for Compound 11. ¹H NMR (400 MHz, CDCl₃) δ 4.45 (t, J=5.7 Hz, 1H), 4.05 (t, J=6.8 Hz, 2H), 3.63 (t, J=5.3 Hz, 2H), 3.56 (dt, J=9.3, 6.7 Hz, 2H), 3.40 (dt, J=9.3, 6.7 Hz, 2H), 2.70 (t, J=5.2 Hz, 2H), 2.58 (t, J=7.7 Hz, 4H), 2.29 (t, J=7.6 Hz, 2H), 1.68-1.17 (m, 65H), 0.88 (t, J=6.7 Hz, 9H). MS: 699.4 m/z [M+H].

### Example 24—Compound 24

### Intermediate 24a: 10-bromodecyl heptanoate

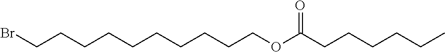 [SMILES: CCCCCCC(=O)OCCCCCCCCCCBr]

Intermediate 24a was synthesized in 26% yield from heptanoic acid and 10-bromodecane-1-ol using the method employed in Intermediate 22a. ¹H NMR (400 MHz, CDCl₃) δ 4.05 (t, J=6.7 Hz, 2H), 3.40 (t, J=6.9 Hz, 2H), 2.29 (t, J=7.5 Hz, 2H), 1.85 (dt, J=14.5, 6.9 Hz, 2H), 1.61 (p, J=7.7, 7.2 Hz, 4H), 1.48-1.23 (m, 18H), 0.93-0.84 (m, 3H).

### Compound 24: 10-((8,8-bis(octyloxy)octyl)(2-hydroxyethyl)amino)decyl heptanoate

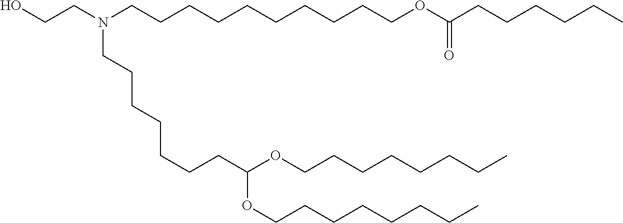 [SMILES: CCCCCCCCOC(CCCCCCCN(CCO)CCCCCCCCCCOC(=O)CCCCCC)OCCCCCCCC]

Compound 24 was synthesized in 40% yield from Intermediate 11a and Intermediate 24a using the method employed for Compound 11. ¹H NMR (400 MHz, CDCl₃) δ 4.45 (t, J=5.7 Hz, 1H), 4.05 (t, J=6.7 Hz, 2H), 3.66-3.50 (m, 4H), 3.40 (dt, J=9.4, 6.7 Hz, 2H), 2.69 (t, J=5.2 Hz, 2H), 2.57 (t, J=7.6 Hz, 4H), 2.29 (t, J=7.5 Hz, 2H), 1.69-0.98 (m, 63H), 0.97-0.70 (m, 9H). MS: 699.6 m/z [M+H].

### Example 25—Compound 25

### Intermediate 25a: 8-bromo-1,1-bis (1-methylheptoxy)octane

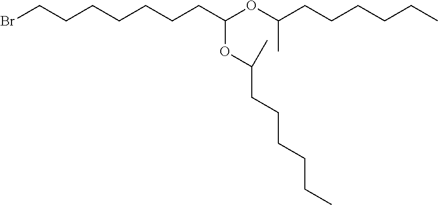 [SMILES: CCCCCCC(C)OC(CCCCCCCBr)OC(C)CCCCCC]

To a solution of 8-bromooctanal (100 mg, 1.0 equiv.) in octan-2-ol (15 equiv.) was added sulfuric acid (0.1 equiv.). The mixture was stirred at 20° C. for 12 h. Upon completion, the reaction mixture was quenched with iced water and extracted 2× with EtOAc. The combined organic layers were concentrated under reduced pressure and purified by column (EtOAc/hexanes) to afford product as a colorless oil (20 mg, 9%). ¹H NMR (400 MHz, CDCl₃) δ 4.44 (td, J=5.6, 3.9 Hz, 1H), 3.64-3.49 (m, 2H), 3.33 (t, J=6.9 Hz, 2H), 1.78 (p, J=7.0 Hz, 2H), 1.60-1.41 (m, 4H), 1.41-1.14 (m, 24H), 1.10 (dd, J=6.2, 2.2 Hz, 3H), 1.03 (d, J=6.1 Hz, 3H), 0.81 (td, J=6.8, 2.5 Hz, 6H).

### Compound 25: nonyl 8-[8,8-bis(1-methylheptoxy)octyl-(2-hydroxyethyl)amino]octanoate

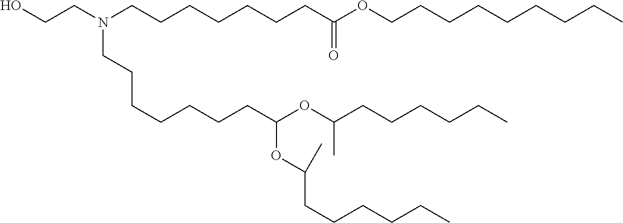 [SMILES: CCCCCCCCCOC(=O)CCCCCCCN(CCO)CCCCCCCC(OC(C)CCCCCC)OC(C)CCCCCC]

Compound 25 was synthesized from Intermediate 1b and Intermediate 25a using the method employed for Compound 1. ¹H NMR (400 MHz, CDCl₃) δ 4.46-4.40 (m, 1H), 3.99 (t, J=6.7 Hz, 2H), 3.57 (tq, J=11.4, 5.9 Hz, 2H), 3.47 (t, J=5.3 Hz, 2H), 2.52 (t, J=5.3 Hz, 2H), 2.39 (t, J=7.5 Hz, 4H), 2.22 (t, J=7.5 Hz, 2H), 1.61-1.42 (m, 7H), 1.41-1.15 (m, 39H), 1.10 (dd, J=6.2, 2.1 Hz, 3H), 1.03 (d, J=6.1 Hz, 3H), 0.81 (t, J=6.5 Hz, 9H). MS: 699.7 m/z [M+H].

### Example 26—Compound 26

### Compound 26: nonyl 8-((2-hydroxyethyl)(10-octyloctadecyl)amino)octanoate

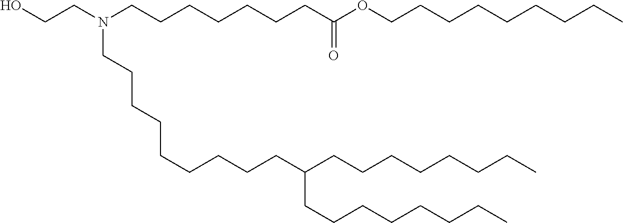 [SMILES: CCCCCCCCCOC(=O)CCCCCCCN(CCO)CCCCCCCCCC(CCCCCCCC)CCCCCCCC]

Compound 26 was synthesized according to methods described in WO 2017/049245 A3 (Example 153). ¹H NMR (400 MHz, CDCl₃) δ 3.99 (t, J=6.7 Hz, 2H), 3.46 (t, J=5.4 Hz, 2H), 2.51 (t, J=5.4 Hz, 2H), 2.38 (t, J=7.5 Hz, 4H), 2.22 (t, J=7.5 Hz, 3H), 1.54 (t, J=7.1 Hz, 5H), 1.37 (t, J=7.2 Hz, 4H), 1.33-1.07 (m, 63H), 0.81 (t, J=6.6 Hz, 9H). MS: 694.6 m/z [M+H].

### Example 27—Compound 27

### Intermediate 27a: octan-2-yl 8-bromooctanoate

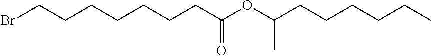 [SMILES: CCCCCCC(C)OC(=O)CCCCCCCBr]

To a mixture of 8-bromooctanoic acid (10 g, 1.1 equiv.) and octan-2-ol (1.0 equiv.) in DCM (150 mL) was added EDCI (1.1 equiv.), DMAP (0.1 equiv.), and DIPEA (3.0 equiv.) in one portion at 0° C. under inert atmosphere. The mixture was stirred at 15° C. for at least 12 h. Upon completion, the reaction mixture was concentrated under reduced pressure, and the resulting crude residue was purified by column chromatography to afford product as a colorless oil (4.1 g, 30%). ¹H NMR (400 MHz, CDCl₃) δ 4.90-4.76 (m, 1H), 3.33 (t, J=6.8 Hz, 2H), 2.20 (t, J=7.5 Hz, 2H), 1.78 (p, J=7.0 Hz, 2H), 1.60-1.46 (m, 3H), 1.39 (dt, J=15.5, 6.6 Hz, 3H), 1.31-1.16 (m, 12H), 1.13 (d, J=6.3 Hz, 3H), 0.86-0.77 (m, 3H).

### Compound 27: octan-2-yl 8-((8,8-bis(octyloxy)octyl)(2-hydroxyethyl)amino)octanoate

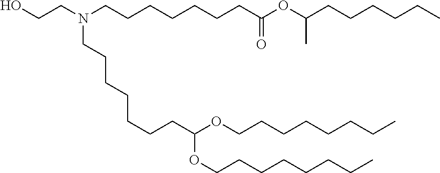 [SMILES: CCCCCCCCOC(CCCCCCCN(CCO)CCCCCCCC(=O)OC(C)CCCCCC)OCCCCCCCC]

Compound 27 was synthesized in 45% yield from Intermediate 11a and Intermediate 27a using the method employed for Compound 11. ¹H NMR (400 MHz, CDCl₃) δ 4.93-4.84 (m, 1H), 4.45 (t, J=5.7 Hz, 1H), 3.78 (s, 2H), 3.55 (dt, J=9.3, 6.6 Hz, 2H), 3.40 (dt, J=9.3, 6.7 Hz, 2H), 2.77 (d, J=52.0 Hz, 5H), 2.27 (t, J=7.5 Hz, 2H), 1.93-1.40 (m, 17H), 1.39-1.21 (m, 37H), 1.19 (d, J=6.3 Hz, 3H), 0.99-0.67 (m, 9H). MS: 685.6 m/z [M+H].

### Example 28—Compound 28

### Intermediate 28a: nonan-3-yl 8-bromooctanoate

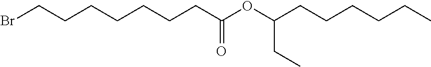 [SMILES: CCCCCCC(CC)OC(=O)CCCCCCCBr]

Intermediate 28a was synthesized in 31% yield from 8-bromooctanoic acid and nonan-3-ol using the method employed for Intermediate 27a. ¹H NMR (400 MHz, CDCl₃) δ 4.75 (p, J=6.2 Hz, 1H), 3.33 (t, J=6.8 Hz, 2H), 2.22 (t, J=7.5 Hz, 2H), 1.78 (p, J=7.0 Hz, 2H), 1.55 (td, J=8.9, 8.2, 5.7 Hz, 2H), 1.47 (dtd, J=14.2, 7.1, 3.2 Hz, 4H), 1.36 (dt, J=10.1, 6.4 Hz, 2H), 1.32-1.12 (m, 12H), 0.88-0.76 (m, 6H).

### Compound 28: nonan-3-yl 8-((8,8-bis(octyloxy)octyl)(2-hydroxyethyl)amino)octanoate

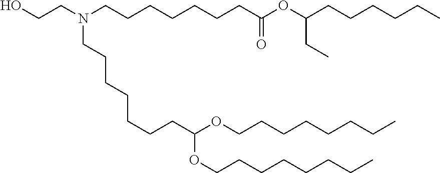 [SMILES: CCCCCCCCOC(CCCCCCCN(CCO)CCCCCCCC(=O)OC(CC)CCCCCC)OCCCCCCCC]

Compound 28 was synthesized in 53% yield from Intermediate 11a and Intermediate 28a using the method employed for Compound 11. ¹H NMR (400 MHz, CDCl₃) δ 4.81 (ddd, J=12.5, 6.9, 5.5 Hz, 1H), 4.45 (t, J=5.7 Hz, 1H), 3.80 (s, 2H), 3.55 (dt, J=9.3, 6.7 Hz, 2H), 3.40 (dt, J=9.4, 6.7 Hz, 2H), 2.81 (s, 5H), 2.29 (t, J=7.4 Hz, 2H), 1.79-1.40 (m, 18H), 1.40-1.02 (m, 42H), 0.95-0.73 (m, 12H). MS: 699.3 m/z [M+H].

### Example 29—Compound 29

### Intermediate 29a: pentyl 8-bromooctanoate

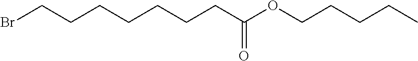 [SMILES: CCCCCOC(=O)CCCCCCCBr]

Intermediate 29a was synthesized in 47% yield from 8-bromooctanoic acid and pentan-1-ol using the method employed for Intermediate 27a. ¹H NMR (400 MHz, CDCl₃) δ 3.99 (td, J=6.8, 1.6 Hz, 2H), 3.33 (td, J=6.8, 1.6 Hz, 2H), 2.23 (t, J=7.5 Hz, 2H), 1.84-1.75 (m, 2H), 1.56 (q, J=7.0 Hz, 4H), 1.47-1.33 (m, 2H), 1.26 (qt, J=5.0, 1.8 Hz, 8H), 0.86-0.80 (m, 3H).

### Compound 29: pentyl 8-((8,8-bis(octyloxy)octyl)(2-hydroxyethyl)amino)octanoate

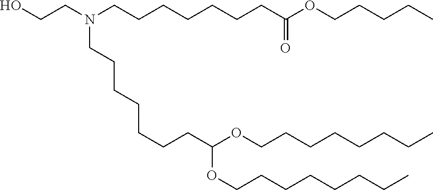 [SMILES: CCCCCCCCOC(CCCCCCCN(CCO)CCCCCCCC(=O)OCCCCC)OCCCCCCCC]

Compound 29 was synthesized in 58% yield from Intermediate 11a and Intermediate 29a using the method employed for Compound 11. ¹H NMR (400 MHz, CDCl₃) δ 4.44 (t, J=5.7 Hz, 1H), 4.06 (t, J=6.8 Hz, 2H), 3.94 (s, 2H), 3.55 (dt, J=9.3, 6.7 Hz, 2H), 3.40 (dt, J=9.3, 6.7 Hz, 2H), 3.03 (d, J=38.0 Hz, 5H), 2.29 (dd, J=8.5, 6.4 Hz, 2H), 1.79 (s, 4H), 1.67-1.41 (m, 14H), 1.41-1.12 (m, 37H), 1.02-0.76 (m, 9H). MS: 643.4 m/z [M+H].

### Example 30—Compound 30

### Intermediate 30a: heptan-3-yl 8-bromooctanoate

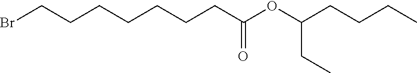 [SMILES: CCCCC(CC)OC(=O)CCCCCCCBr]

Intermediate 30a was synthesized in 47% yield from 8-bromooctanoic acid and heptan-3-ol using the method employed for Intermediate 27a.

### Compound 30: heptan-3-yl 8-((8,8-bis(octyloxy)octyl)(2-hydroxyethyl)amino)octanoate

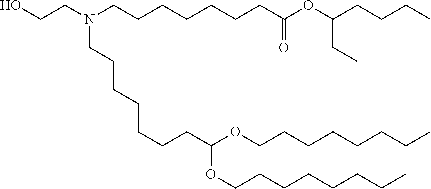 [SMILES: CCCCCCCCOC(CCCCCCCN(CCO)CCCCCCCC(=O)OC(CC)CCCC)OCCCCCCCC]

Compound 30 was synthesized in 66% yield from Intermediate 11a and Intermediate 30a using the method employed for Compound 11. ¹H NMR (400 MHz, CDCl₃) δ 4.81 (ddd, J=12.5, 6.8, 5.5 Hz, 1H), 4.45 (t, J=5.8 Hz, 1H), 3.77 (d, J=53.2 Hz, 2H), 3.55 (dt, J=9.3, 6.7 Hz, 2H), 3.40 (dt, J=9.3, 6.7 Hz, 2H), 2.71 (s, 5H), 2.29 (t, J=7.5 Hz, 2H), 1.83-1.44 (m, 17H), 1.30 (dq, J=18.1, 3.8, 3.2 Hz, 37H), 1.02-0.69 (m, 12H). MS: 671.5 m/z [M+H].

### Example 31—Compound 31

### Intermediate 31a: 2-((7,7-bis(octyloxy)heptyl)amino)ethan-1-ol

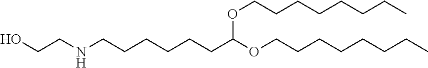 [SMILES: CCCCCCCCOC(CCCCCCNCCO)OCCCCCCCC]

To a solution of Intermediate 10b (15 g, 1.0 equiv.) in EtOH (22 mL) was added 2-aminoethanol (30 equiv.). The mixture was stirred at 15° C. for 12 h. Upon completion, the reaction was mixture was concentrated under reduced pressure to afford a residue that was purified by column chromatography. After fractions containing product were concentrated, the resulting residue was reconstituted in MeCN and extracted 3× with hexane. The combined hexane layers were concentrated to afford product as a colorless oil (10.55 g, 73% yield). ¹H NMR (400 MHz, CDCl₃) δ 4.47 (t, J=5.7 Hz, 1H), 3.68-3.62 (m, 2H), 3.58 (dt, J=9.3, 6.6 Hz, 2H), 3.42 (dt, J=9.4, 6.7 Hz, 2H), 2.82-2.76 (m, 2H), 2.63 (t, J=7.1 Hz, 2H), 1.67-1.45 (m, 8H), 1.45-1.19 (m, 26H), 0.96-0.84 (m, 6H).

### Compound 31: heptyl 8-((7,7-bis(octyloxy)heptyl)(2-hydroxyethyl)amino)octanoate

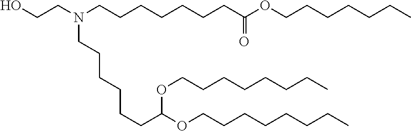 [SMILES: CCCCCCCCOC(CCCCCCN(CCO)CCCCCCCC(=O)OCCCCCCC)OCCCCCCCC]

Compound 31 was synthesized in 68% yield from Intermediate 31a and Intermediate 15a using the method employed for Compound 11. ¹H NMR (500 MHz, CDCl₃) δ 4.45 (t, J=5.7 Hz, 1H), 4.06 (t, J=6.8 Hz, 2H), 3.73 (s, 2H), 3.55 (dt, J=9.3, 6.7 Hz, 2H), 3.40 (dt, J=9.4, 6.7 Hz, 2H), 2.58 (d, J=135.2 Hz, 6H), 2.29 (t, J=7.5 Hz, 2H), 1.59 (ddt, J=21.1, 14.3, 6.8 Hz, 15H), 1.44-1.02 (m, 40H), 0.88 (td, J=7.0, 2.9 Hz, 9H). MS: 657.4 m/z [M+H].

### Example 32—Compound 32

### Compound 32: octan-2-yl 8-((7,7-bis(octyloxy)heptyl)(2-hydroxyethyl)amino)octanoate

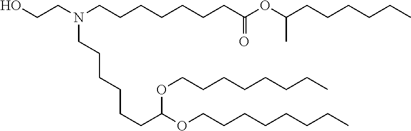 [SMILES: CCCCCCCCOC(CCCCCCN(CCO)CCCCCCCC(=O)OC(C)CCCCCC)OCCCCCCCC]

Compound 32 was synthesized in 64% yield from Intermediate 31a and Intermediate 27a using the method employed for Compound 11. ¹H NMR (500 MHz, CDCl₃) δ 4.89 (h, J=6.3 Hz, 1H), 4.45 (t, J=5.7 Hz, 1H), 3.55 (dt, J=9.4, 6.8 Hz, 5H), 3.40 (dt, J=9.3, 6.8 Hz, 2H), 3.07-2.32 (m, 7H), 2.27 (t, J=7.5 Hz, 2H), 1.79-1.40 (m, 15H), 1.40-1.22 (m, 38H), 1.19 (d, J=6.2 Hz, 3H), 0.88 (t, J=6.8 Hz, 9H). MS: 671.4 m/z [M+H].

### Example 33—Compound 33

### Compound 33: nonan-3-yl 8-((7,7-bis(octyloxy)heptyl)(2-hydroxyethyl)amino)octanoate

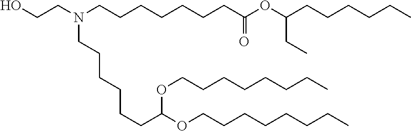 [SMILES: CCCCCCCCOC(CCCCCCN(CCO)CCCCCCCC(=O)OC(CC)CCCCCC)OCCCCCCCC]

Compound 33 was synthesized in 60% yield from Intermediate 31a and Intermediate 28a using the method employed for Compound 11. ¹H NMR (500 MHz, CDCl₃) δ 4.81 (p, J=6.3 Hz, 1H), 4.45 (t, J=5.7 Hz, 1H), 3.55 (dt, J=9.3, 6.7 Hz, 4H), 3.40 (dt, J=9.3, 6.7 Hz, 2H), 2.89-2.40 (m, 5H), 2.29 (t, J=7.5 Hz, 2H), 1.57 (dtt, J=21.7, 14.5, 6.4 Hz, 14H), 1.41-1.08 (m, 35H), 0.88 (td, J=7.1, 2.8 Hz, 10H). MS: 685.7 m/z [M+H].

### Example 34—Compound 34

### Compound 34: pentyl 8-((7,7-bis(octyloxy)heptyl)(2-hydroxyethyl)amino)octanoate

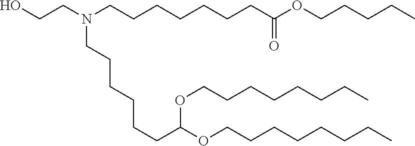 [SMILES: CCCCCCCCOC(CCCCCCN(CCO)CCCCCCCC(=O)OCCCCC)OCCCCCCCC]

Compound 34 was synthesized in 72% yield from Intermediate 31a and Intermediate 29a using the method employed for Compound 11. ¹H NMR (400 MHz, CDCl₃) δ 4.45 (t, J=5.7 Hz, 1H), 4.05 (t, J=6.7 Hz, 2H), 3.75 (s, 2H), 3.55 (dt, J=9.3, 6.7 Hz, 2H), 3.39 (dt, J=9.3, 6.7 Hz, 2H), 2.94-2.40 (m, 6H), 2.29 (t, J=7.5 Hz, 2H), 1.83-1.43 (m, 15H), 1.42-1.09 (m, 36H), 0.89 (dt, J=11.2, 7.0 Hz, 9H). MS: 629.4 m/z [M+H].

### Example 35—Compound 35

### Compound 35: heptan-3-yl 8-((7,7-bis(octyloxy)heptyl)(2-hydroxyethyl)amino)octanoate

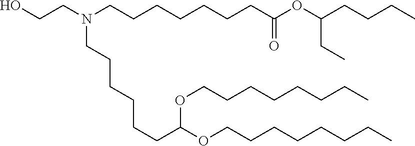 [SMILES: CCCCCCCCOC(CCCCCCN(CCO)CCCCCCCC(=O)OC(CC)CCCC)OCCCCCCCC]

Compound 35 was synthesized in 73% yield from Intermediate 31a and Intermediate 30a using the method employed for Compound 11. ¹H NMR (400 MHz, CDCl₃) δ 4.85-4.78 (m, 1H), 4.45 (t, J=5.7 Hz, 1H), 3.68 (s, 2H), 3.55 (dt, J=9.3, 6.7 Hz, 2H), 3.39 (dt, J=9.3, 6.7 Hz, 2H), 2.86-2.37 (m, 6H), 2.29 (t, J=7.5 Hz, 2H), 1.53 (dtd, J=14.4, 7.4, 5.6 Hz, 16H), 1.43-1.08 (m, 37H), 0.97-0.80 (m, 12H). MS: 657.6 m/z [M+H].

### Example 36—Compound 36

### Compound 36: nonyl 8-((2-aminoethyl)(7,7-bis(octyloxy)heptyl)amino)octanoate

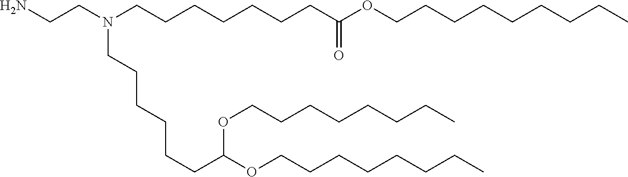 [SMILES: CCCCCCCCCOC(=O)CCCCCCCN(CCN)CCCCCCC(OCCCCCCCC)OCCCCCCCC]

To a mixture of Compound 10 (5.1 g, 1.0 equiv.) and TEA (1.35 mL, 1.3 equiv.) in DCM (50 mL) was added MsCl (721 uL, 1.25 equiv.) drop wise at 0° C. under inert atmosphere. The mixture was stirred at 15° C. for 12 h. TLC indicated starting material was completely consumed. The reaction was diluted with H₂O and extracted 2× with DCM, dried over Na₂SO₄, filtered, and the filtrate was concentrated under reduced pressure to give a residue.

The resulting crude mesylate was dissolved in DMF (60 mL) followed by the addition of NaN₃ (2.78 g, 5.0 equiv.) in one portion at 15° C. under inert atmosphere. The mixture was stirred at 100° C. for 4 h. TLC indicated complete displacement. The reaction mixture was diluted with H₂O and extracted 2× with EtOAc, dried over Na₂SO₄, filtered and the filtrate was concentrated under reduced pressure to give a residue.

The resulting crude azide was dissolved in EtOH (5 mL) followed by the addition of Pd/C (1 g, 10% w/w) under inert atmosphere. The suspension was degassed under vacuum and purged with H₂ several times. The mixture was stirred under H₂ (15 psi) at 15° C. for 12 h. Upon completion, the reaction mixture was filtered and the filtrate was concentrated under reduced pressure to give a residue. The residue was purified by column chromatography three times before the isolated material was washed with MeCN and hexanes to afford product as a yellow oil (2.3 g, 39%). ¹H NMR (400 MHz, CDCl₃) δ 4.80 (s, 3H), 4.38 (t, J=5.7 Hz, 1H), 3.98 (t, J=6.8 Hz, 2H), 3.48 (dt, J=9.4, 6.7 Hz, 2H), 3.33 (dt, J=9.5, 6.8 Hz, 2H), 2.82 (t, J=5.9 Hz, 2H), 2.57 (t, J=6.0 Hz, 2H), 2.50-2.36 (m, 4H), 2.22 (t, J=7.5 Hz, 2H), 1.62-1.33 (m, 15H), 1.33-1.04 (m, 45H), 0.81 (t, J=6.6 Hz, 9H). MS: 683.6 m/z [M+H].

### Example 37—Compound 37

### Intermediate 37a: nonyl 8-((3-hydroxypropyl)amino)octanoate

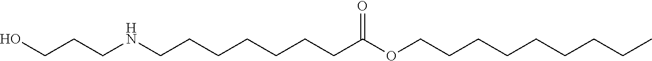 [SMILES: CCCCCCCCCOC(=O)CCCCCCCNCCCO]

A mixture of nonyl 8-bromooctanoate (10 g, 1.0 equiv.) and 3-aminopropan-1-ol (66.22 mL, 30 equiv.) in EtOH (15 mL) was stirred at 20° C. for 12 hours. Upon completion, the reaction mixture was concentrated under reduced pressure to give a residue. The residue was purified by column chromatography to afford product (10 g) as a colorless oil. ¹H NMR (400 MHz, CDCl₃) δ 4.07 (t, J=6.7 Hz, 2H), 3.84 (dt, J=10.5, 5.4 Hz, 2H), 3.66 (t, J=5.6 Hz, 6H), 3.43 (q, J=6.2 Hz, 6H), 2.89 (t, J=5.6 Hz, 2H), 2.61 (t, J=7.1 Hz, 2H), 2.30 (t, J=7.5 Hz, 2H), 2.03 (s, 10H), 1.66 (dt, J=29.5, 6.6 Hz, 14H), 1.47 (t, J=7.0 Hz, 3H), 1.31 (d, J=14.6 Hz, 16H), 0.90 (t, J=6.6 Hz, 3H).

### Compound 37: nonyl 8-((7,7-bis(octyloxy)heptyl)(3-hydroxypropyl)amino)octanoate

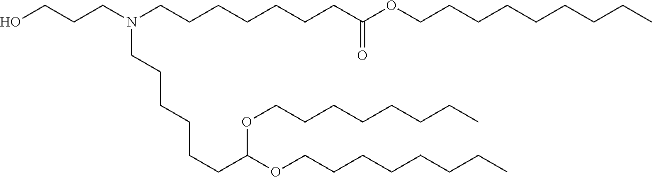 [SMILES: CCCCCCCCCOC(=O)CCCCCCCN(CCCO)CCCCCCC(OCCCCCCCC)OCCCCCCCC]

Compound 37 was synthesized in 30% yield from Intermediate 1b and Intermediate 37a using the method employed for Compound 1. ¹H NMR (400 MHz, CDCl₃) δ 4.47 (t, J=5.7 Hz, 2H), 4.08 (t, J=6.7 Hz, 2H), 3.81 (t, J=5.1 Hz, 2H), 3.60-3.55 (m, 2H), 3.42 (dt, J=9.3, 6.7 Hz, 3H), 2.68-2.62 (m, 2H), 2.47-2.38 (m, 4H), 2.31 (t, J=7.5 Hz, 2H), 1.61 (dt, J=21.4, 7.2 Hz, 21H), 1.31 (dt, J=15.0, 4.1 Hz, 51H), 0.90 (t, J=6.7 Hz, 9H). MS: 698.7 m/z [M+H].

### Example 38—Compound 38

### Compound 38: nonyl 8-((3-aminopropyl)(7,7-bis(octyloxy)heptyl)amino)octanoate

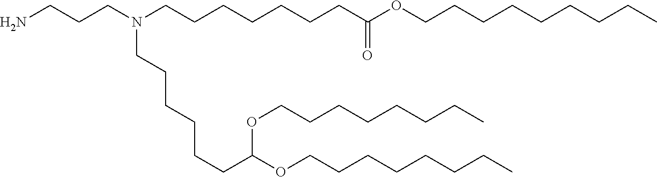 [SMILES: CCCCCCCCCOC(=O)CCCCCCCN(CCCN)CCCCCCC(OCCCCCCCC)OCCCCCCCC]

Compound 38 was synthesized from Compound 37 using the method employed for Compound 36. ¹H NMR (400 MHz, CDCl₃) δ 4.38 (t, J=5.7 Hz, 1H), 3.98 (t, J=6.8 Hz, 2H), 3.48 (dt, J=9.5, 6.7 Hz, 2H), 3.33 (dt, J=9.4, 6.7 Hz, 2H), 2.77 (q, J=5.2, 4.0 Hz, 2H), 2.48 (t, J=6.9 Hz, 2H), 2.43-2.31 (m, 4H), 2.22 (t, J=7.5 Hz, 2H), 1.54 (dtd, J=28.3, 13.8, 6.7 Hz, 12H), 1.43-1.11 (m, 49H), 0.81 (t, J=6.7 Hz, 9H). MS: 697.8 m/z [M+H].

### Example 39—Compound 39

### Compound 39: nonyl 8-((7,7-bis(octyloxy)heptyl)(2-((methylcarbamoyl)oxy)ethyl) amino)octanoate

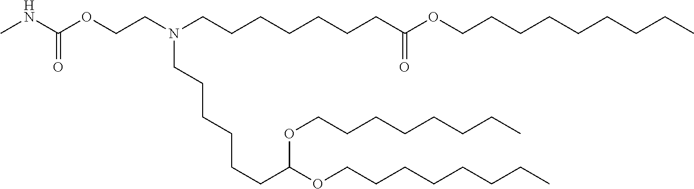 [SMILES: CCCCCCCCCOC(=O)CCCCCCCN(CCCCCCC(OCCCCCCCC)OCCCCCCCC)CCOC(=O)NC]

To a mixture of Compound 10 (1.0 equiv.) in toluene (0.1 M) was added methyl isocyanate (1.4 equiv.). The reaction was stirred for 24 h at 23° C., followed by 48 h at 60° C. Upon completion, the reaction was diluted with water and extracted 3× with DCM. The combined organic layers were concentrated and purified by column chromatography to afford product (33%). ¹H NMR (500 MHz, CDCl₃) δ 4.44 (t, J=5.7 Hz, 1H), 4.19 (s, 1H), 4.05 (t, J=6.7 Hz, 2H), 3.55 (dt, J=9.3, 6.7 Hz, 2H), 3.39 (dt, J=9.3, 6.7 Hz, 2H), 2.79 (d, J=4.9 Hz, 3H), 2.28 (t, J=7.5 Hz, 2H), 1.58 (dp, J=21.1, 7.0 Hz, 13H), 1.31 (ddd, J=23.5, 12.5, 5.9 Hz, 46H), 0.88 (t, J=6.8 Hz, 9H). MS: 742.7 m/z [M+H].

### Example 40—Compound 40

### Compound 40: nonyl 8-((7,7-bis(octyloxy)heptyl)(3-((methylcarbamoyl)oxy)propyl)amino)octanoate

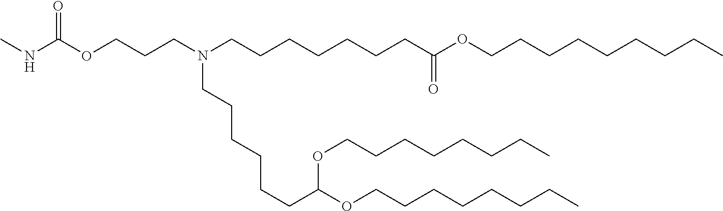 [SMILES: CCCCCCCCCOC(=O)CCCCCCCN(CCCCCCC(OCCCCCCCC)OCCCCCCCC)CCCOC(=O)NC]

Compound 40 was synthesized in 34% yield from Compound 37 using the method employed for Compound 39. ¹H NMR (500 MHz, CDCl₃) δ 4.45 (t, J=5.7 Hz, 1H), 4.10 (t, J=6.4 Hz, 2H), 4.05 (t, J=6.8 Hz, 2H), 3.57-3.52 (m, 2H), 3.40 (dt, J=9.4, 6.8 Hz, 2H), 2.79 (d, J=4.8 Hz, 4H), 2.37 (s, 4H), 2.29 (t, J=7.5 Hz, 2H), 1.58 (dp, J=21.2, 7.0 Hz, 13H), 1.30 (ddt, J=17.9, 11.6, 5.7 Hz, 49H), 0.88 (t, J=6.8 Hz, 9H). MS: 756.4 m/z [M+H].

### Example 41—Compound 41

### Compound 41: nonyl 8-((2-acetamidoethyl)(7,7-bis(octyloxy)heptyl)amino)octanoate

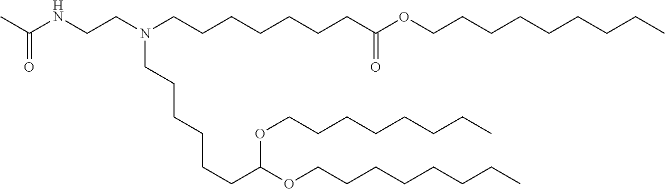 [SMILES: CCCCCCCCCOC(=O)CCCCCCCN(CCCCCCC(OCCCCCCCC)OCCCCCCCC)CCNC(C)=O]

To a mixture of Compound 36 (1.0 equiv.) in DCM (0.2 M) was added TEA (1.1 equiv.) and cooled to 0° C. Acetyl chloride (1.04 equiv.) was added dropwise, and the mixture was stirred for 4 h. Upon completion, the reaction was quenched with sat. sodium bicarb solution and extracted 3× with DCM. The combined organic layers were concentrated and purified by column chromatography to afford product as a colorless oil (55%). ¹H NMR (400 MHz, CDCl₃) δ 4.44 (t, J=5.7 Hz, 1H), 4.05 (t, J=6.7 Hz, 2H), 3.55 (dt, J=9.3, 6.7 Hz, 2H), 3.42-3.37 (m, 2H), 3.35 (d, J=13.8 Hz, 2H), 2.56 (d, J=48.6 Hz, 5H), 2.29 (t, J=7.5 Hz, 2H), 1.98 (s, 3H), 1.66-1.41 (m, 15H), 1.41-1.20 (m, 48H), 0.90-0.83 (m, 9H). MS: 740.9 m/z [M+H].

### Example 42—Compound 42

### Compound 42: nonyl 8-((3-acetamidopropyl)(7,7-bis(octyloxy)heptyl)amino)octanoate

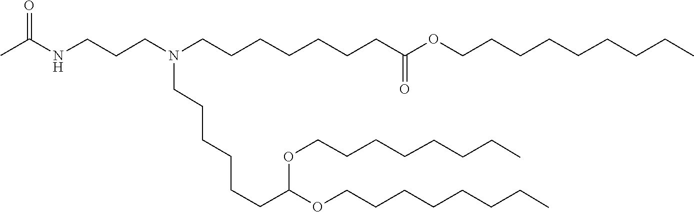 [SMILES: CCCCCCCCCOC(=O)CCCCCCCN(CCCCCCC(OCCCCCCCC)OCCCCCCCC)CCCNC(C)=O]

Compound 42 was synthesized in 51% yield from Compound 38 using the method employed for Compound 41. ¹H NMR (400 MHz, CDCl₃) δ 4.44 (t, J=5.7 Hz, 1H), 4.05 (t, J=6.7 Hz, 2H), 3.55 (dt, J=9.4, 6.7 Hz, 2H), 3.39 (dt, J=9.3, 6.7 Hz, 2H), 3.32 (q, J=5.6 Hz, 2H), 2.52 (t, J=6.0 Hz, 2H), 2.46-2.35 (m, 3H), 2.29 (t, J=7.5 Hz, 2H), 1.93 (s, 3H), 1.68-1.50 (m, 12H), 1.44 (h, J=6.9, 6.1 Hz, 4H), 1.39-1.21 (m, 45H), 0.92-0.84 (m, 9H). MS: 742.7 m/z [M+H].

### Example 43—Compound 43

### Compound 43: nonyl 8-((7,7-bis(octyloxy)heptyl)(3-((methoxycarbonyl)amino)propyl)amino)octanoate

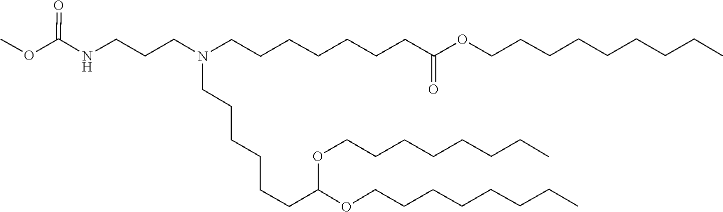 [SMILES: CCCCCCCCCOC(=O)CCCCCCCN(CCCCCCC(OCCCCCCCC)OCCCCCCCC)CCCNC(=O)OC]

To a mixture of Compound 38 (1.0 equiv.) in DCM (0.2 M) and TEA (1.1 equiv.) was cooled to 0° C. Methyl chloroformate (1.1 equiv.) was added dropwise, and the mixture was stirred for 4 h. Upon completion, the reaction was quenched with sat. sodium bicarb solution and extracted 3× with DCM. The combined organic layers were concentrated and purified by column chromatography to afford product as a colorless oil (31%). ¹H NMR (400 MHz, CDCl₃) δ 4.45 (t, J=5.7 Hz, 1H), 4.05 (t, J=6.8 Hz, 2H), 3.64 (s, 3H), 3.55 (dt, J=9.3, 6.7 Hz, 2H), 3.40 (dt, J=9.4, 6.7 Hz, 2H), 3.26 (q, J=6.1 Hz, 2H), 2.41 (d, J=44.1 Hz, 4H), 2.29 (t, J=7.5 Hz, 2H), 1.68-1.50 (m, 13H), 1.50-1.20 (m, 49H), 0.93-0.83 (m, 9H). MS: 756.0 m/z [M+H].

### Example 44—Compound 44

### Compound 44: nonyl 8-((7,7-bis(octyloxy)heptyl)(2-((methoxycarbonyl)amino)ethyl)amino)octanoate

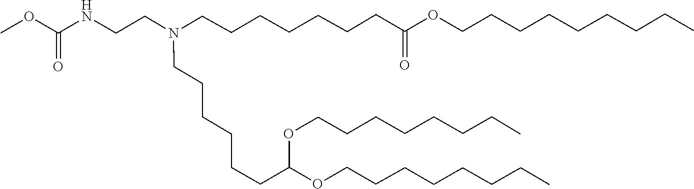 [SMILES: CCCCCCCCCOC(=O)CCCCCCCN(CCCCCCC(OCCCCCCCC)OCCCCCCCC)CCNC(=O)OC]

Compound 44 was synthesized in 56% yield from Compound 36 using the method employed for Compound 43. ¹H NMR (400 MHz, CDCl₃) δ 4.45 (t, J=5.7 Hz, 1H), 4.05 (t, J=6.7 Hz, 2H), 3.66 (s, 3H), 3.58-3.50 (m, 2H), 3.40 (dt, J=9.4, 6.7 Hz, 2H), 3.21 (s, 2H), 2.44 (d, J=49.2 Hz, 5H), 2.29 (t, J=7.5 Hz, 2H), 1.65-1.50 (m, 11H), 1.48-1.16 (m, 52H), 0.91-0.83 (m, 9H). MS: 742.4 m/z [M+H].

### Example 45—Compound 45

### Compound 45: nonyl 8-((7,7-bis(octyloxy)heptyl)(2-(3-methylureido)ethyl)amino)octanoate

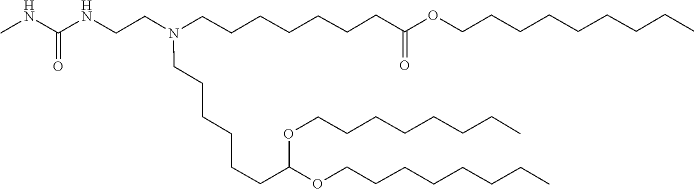 [SMILES: CCCCCCCCCOC(=O)CCCCCCCN(CCCCCCC(OCCCCCCCC)OCCCCCCCC)CCNC(=O)NC]

To a mixture of Compound 36 (1.0 equiv.) in toluene (0.02 M) was added methyl isocyanate (1.4 equiv.). The reaction was stirred for 4 h at 23° C. Upon completion, the reaction was diluted with water and extracted 3× with DCM. The combined organic layers were concentrated and purified by column chromatography to afford product (23%). ¹H NMR (400 MHz, CDCl₃) δ 5.20 (s, 1H), 4.45 (t, J=5.7 Hz, 1H), 4.05 (t, J=6.8 Hz, 2H), 3.55 (dt, J=9.3, 6.7 Hz, 2H), 3.40 (dt, J=9.3, 6.7 Hz, 2H), 3.27 (d, J=18.4 Hz, 2H), 2.75 (d, J=4.8 Hz, 3H), 2.54 (d, J=51.5 Hz, 5H), 2.29 (t, J=7.5 Hz, 2H), 1.67-1.40 (m, 15H), 1.40-1.19 (m, 46H), 0.92-0.84 (m, 9H). MS: 741.3 m/z [M+H].

### Example 46—Compound 46

### Compound 46: nonyl 8-((7,7-bis(octyloxy)heptyl)(3-(3-methylureido)propyl)amino)octanoate

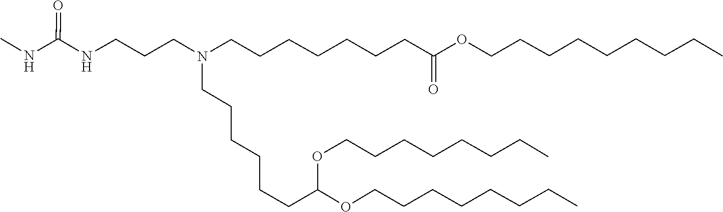 [SMILES: CCCCCCCCCOC(=O)CCCCCCCN(CCCCCCC(OCCCCCCCC)OCCCCCCCC)CCCNC(=O)NC]

Compound 46 was synthesized in 24% yield from Compound 38 using the method employed for Compound 45. ¹H NMR (400 MHz, CDCl₃) δ 4.45 (t, J=5.7 Hz, 1H), 4.05 (t, J=6.7 Hz, 2H), 3.55 (dt, J=9.3, 6.7 Hz, 2H), 3.40 (dt, J=9.3, 6.7 Hz, 2H), 3.25 (h, J=5.0 Hz, 2H), 2.75 (d, J=4.8 Hz, 3H), 2.48 (s, 5H), 2.29 (t, J=7.5 Hz, 2H), 1.73-1.40 (m, 16H), 1.40-1.19 (m, 44H), 0.92-0.83 (m, 9H). MS: 755.0 m/z [M+H].

### Example 47—Compound 47

### Compound 47: nonyl 8-((7,7-bis(octyloxy)heptyl)(2-(methylsulfonamido)ethyl)amino)octanoate

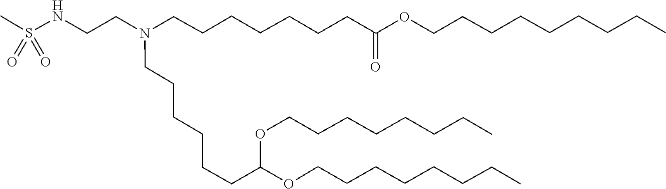 [SMILES: CCCCCCCCCOC(=O)CCCCCCCN(CCCCCCC(OCCCCCCCC)OCCCCCCCC)CCNS(C)(=O)=O]

To a mixture of Compound 36 (1.0 equiv.) in DCM (0.25 M) was added MsCl (10 equiv.). The mixture was stirred for 15 min at 23° C. before being washed 2× with water and concentrated in vacuo. Purification by column chromatography afforded product as a colorless residue (13%). ¹H NMR (400 MHz, CDCl₃) δ 4.47 (t, J=5.7 Hz, 1H), 4.08 (t, J=6.8 Hz, 2H), 3.58 (dt, J=9.3, 6.7 Hz, 2H), 3.42 (dt, J=9.4, 6.7 Hz, 2H), 3.22 (s, 1H), 2.98 (s, 3H), 2.60 (d, J=77.4 Hz, 4H), 2.31 (t, J=7.5 Hz, 2H), 1.69-1.42 (m, 15H), 1.42-1.23 (m, 46H), 0.94-0.86 (m, 9H). MS: 762.9 m/z [M+H].

### Example 48—Compound 48

### Compound 48: nonyl 8-((7,7-bis(octyloxy)heptyl)(3-(methylsulfonamido)propyl)amino)octanoate

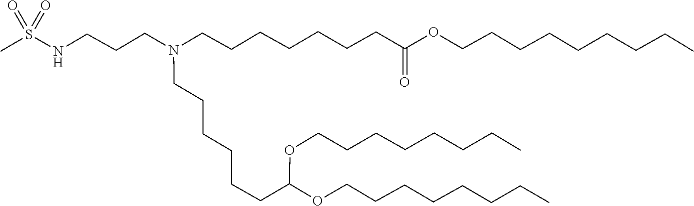 [SMILES: CCCCCCCCCOC(=O)CCCCCCCN(CCCCCCC(OCCCCCCCC)OCCCCCCCC)CCCNS(C)(=O)=O]

Compound 48 was synthesized in 16% yield from Compound 38 using the method employed in Compound 47. ¹H NMR (400 MHz, CDCl₃) δ 4.44 (t, J=5.6 Hz, 1H), 4.05 (t, J=6.8 Hz, 2H), 3.55 (dt, J=9.3, 6.6 Hz, 2H), 3.43-3.32 (m, 4H), 2.97 (s, 7H), 2.29 (t, J=7.4 Hz, 2H), 2.09 (s, 2H), 1.88-1.50 (m, 15H), 1.43-1.18 (m, 41H), 0.97-0.76 (m, 9H). MS: 776.5 m/z [M+H].

### Example 49—Compound 49

### Compound 49: nonyl 8-((2-acetoxyethyl)(7,7-bis(octyloxy)heptyl)amino)octanoate

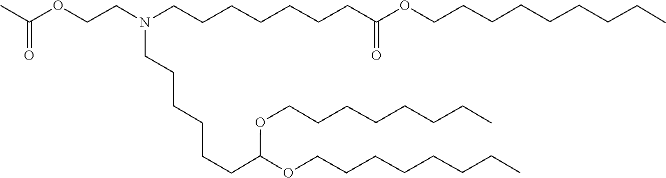 [SMILES: CCCCCCCCCOC(=O)CCCCCCCN(CCCCCCC(OCCCCCCCC)OCCCCCCCC)CCOC(C)=O]

To a mixture of Compound 10 (1.0 equiv.) in pyridine (10 equiv.) was added acetic anhydride (10 equiv.). The mixture was stirred at 23° C. for 24 h. Upon completion, the reaction was quenched by the addition of water and extracted 3× with DCM. The combined organic layers were concentrated under vacuum and purified by column chromatography to afford product as a colorless oil (55%). ¹H NMR (400 MHz, CDCl₃) δ 4.44 (t, J=5.7 Hz, 1H), 4.14 (q, J=5.8, 5.4 Hz, 2H), 4.05 (t, J=6.8 Hz, 2H), 3.55 (dt, J=9.3, 6.7 Hz, 2H), 3.39 (dt, J=9.3, 6.7 Hz, 2H), 2.74 (s, 2H), 2.49 (s, 4H), 2.28 (t, J=7.5 Hz, 2H), 2.05 (s, 3H), 1.66-1.50 (m, 11H), 1.50-1.39 (m, 4H), 1.39-1.19 (m, 48H), 0.91-0.84 (m, 9H). MS: 727.4 m/z [M+H].

### Example 50—Compound 50

### Compound 50: nonyl 8-((3-acetoxypropyl)(7,7-bis(octyloxy)heptyl)amino)octanoate

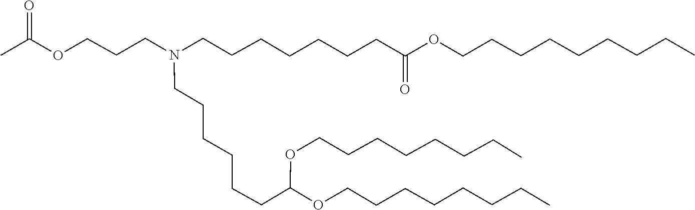 [SMILES: CCCCCCCCCOC(=O)CCCCCCCN(CCCCCCC(OCCCCCCCC)OCCCCCCCC)CCCOC(C)=O]

Compound 50 was synthesized in 42% yield from Compound 37 using the method employed in Compound 49. ¹H NMR (400 MHz, CDCl₃) δ 4.45 (t, J=5.7 Hz, 1H), 4.07 (dt, J=17.3, 6.6 Hz, 4H), 3.55 (dt, J=9.3, 6.6 Hz, 2H), 3.39 (dt, J=9.3, 6.7 Hz, 2H), 2.42 (d, J=38.1 Hz, 5H), 2.28 (t, J=7.5 Hz, 2H), 2.04 (s, 3H), 1.75 (s, 2H), 1.66-1.49 (m, 11H), 1.45-1.21 (m, 52H), 0.91-0.84 (m, 9H). MS: 741.2 m/z [M+H].

### Example 51—pKa Measurements

The pKa of each amine lipid was determined according to the method in Jayaraman, et al. (Angewandte Chemie, 2012) with the following adaptations. The pKa was determined for unformulated amine lipid in ethanol at a concentration of 2.94 mM. Lipid was diluted to 100 μM in 0.1 M phosphate buffer (Boston Bioproducts) where the pH ranged from 4.5-9.0. Fluorescence intensity was measured using excitation and emission wavelengths of 321 nm and 448 nm. Table 2 shows pKa measurements for listed compounds.

TABLE 2
pKa values

| Compound | pKA |
|---|---|
| Compound 1 | 6.30 |
| Compound 2 | 6.23 |
| Compound 3 | 6.19 |
| Compound 4 | 7.04 |
| Compound 5 | 7.08 |
| Compound 6 | 6.3 |
| Compound 7 | 6.16 |
| Compound 8 | 6.37 |
| Compound 9 | 6.12 |
| Compound 10 | 6.05 |
| Compound 11 | 6.35 |
| Compound 12 | 6.38 |
| Compound 13 | 6.35 |
| Compound 14 | 6.46 |
| Compound 15 | 6.34 |
| Compound 16 | 6.34 |
| Compound 17 | 6.21 |
| Compound 18 | 6.6 |
| Compound 19 | 6.4 |
| Compound 20 | 5.81 |
| Compound 21 | 5.99 |
| Compound 22 | 6.18 |
| Compound 23 | 6.04 |
| Compound 24 | 5.95 |
| Compound 25 | 6.1 |
| Compound 26 | 6.19 |
| Compound 27 | 6.41 |
| Compound 28 | 6.41 |
| Compound 29 | 6.54 |
| Compound 30 | 6.48 |
| Compound 31 | 6.47 |
| Compound 32 | 6.36 |
| Compound 33 | 6.33 |
| Compound 34 | 6.47 |
| Compound 35 | 6.37 |
| Compound 36 | 7.2 |
| Compound 37 | 6.3 |
| Compound 38 | 7.81 |
| Compound 39 | undetermined |
| Compound 40 | 5.71 |
| Compound 41 | 6.82 |
| Compound 42 | 7.19 |
| Compound 43 | 6.02 |
| Compound 44 | 5.62 |
| Compound 45 | undetermined |
| Compound 46 | 7.64 |
| Compound 47 | 6.93 |
| Compound 48 | 7.61 |
| Compound 49 | 5.32 |
| Compound 50 | 5.45 |

### Example 52—LNP Compositions for In Vivo Editing in Mice

Preparations of various LNP compositions were prepared with amine lipids. In assays for percent liver editing in mice, Cas9 mRNA and chemically modified sgRNA were formulated in LNPs, at either a 1:1 w/w ratio or a 1:2 w/w ratio. LNPs are formulated with a composition of a given ionizable lipid (e.g. an amine lipid), DSPC, cholesterol, and PEG-2k-DMG, with a 6.0 N:P ratio.

LNP Formulation—Cross Flow

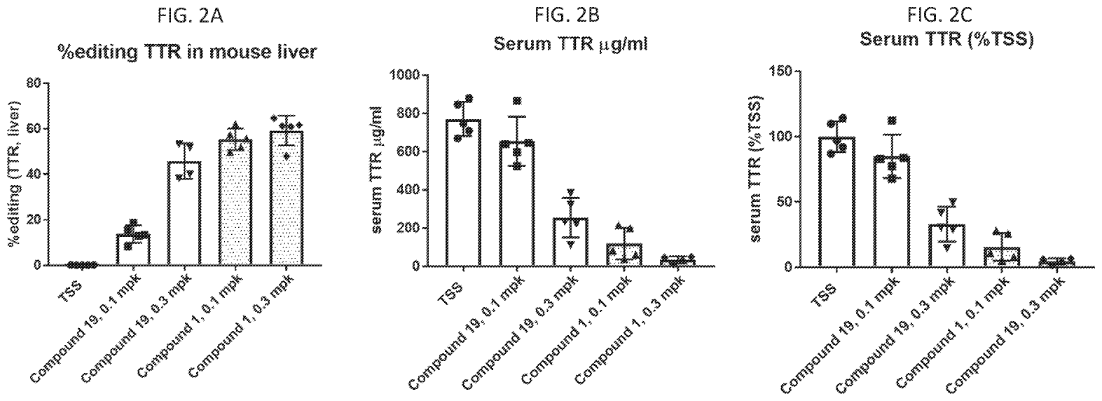

The LNPs were formed by impinging jet mixing of the lipid in ethanol with two volumes of RNA solutions and one volume of water. The lipid in ethanol is mixed through a mixing cross with the two volumes of RNA solution. A fourth stream of water is mixed with the outlet stream of the cross through an inline tee. (See, e.g., WO2016010840, FIG. 2.) The LNPs were held for 1 hour at room temperature, and further diluted with water (approximately 1:1 v/v). Diluted LNPs were concentrated using tangential flow filtration on a flat sheet cartridge (Sartorius, 100 kD MWCO) and then buffer exchanged by diafiltration into 50 mM Tris, 45 mM NaCl, 5% (w/v) sucrose, pH 7.5 (TSS). Alternatively, the final buffer exchange into TSS was completed with PD-10 desalting columns (GE). If required, compositions were concentrated by centrifugation with Amicon 100 kDa centrifugal filters (Millipore). The resulting mixture was then filtered using a 0.2 m sterile filter. The final LNP was stored at 4° C. or −80° C. until further use.

LNP Composition Analytics

Dynamic Light Scattering (“DLS”) is used to characterize the polydispersity index (“pdi”) and size of the LNPs of the present disclosure. DLS measures the scattering of light that results from subjecting a sample to a light source. PDI, as determined from DLS measurements, represents the distribution of particle size (around the mean particle size) in a population, with a perfectly uniform population having a PDI of zero.

Electropheretic light scattering is used to characterize the surface charge of the LNP at a specified pH. The surface charge, or the zeta potential, is a measure of the magnitude of electrostatic repulsion/attraction between particles in the LNP suspension.

Asymmetric-Flow Field Flow Fractionation-Multi-Angle Light Scattering (AF4-MALS) is used to separate particles in the composition by hydrodynamic radius and then measure the molecular weights, hydrodynamic radii and root mean square radii of the fractionated particles. This allows the ability to assess molecular weight and size distributions as well as secondary characteristics such as the Burchard-Stockmeyer Plot (ratio of root mean square (“rms”) radius to hydrodynamic radius over time suggesting the internal core density of a particle) and the rms conformation plot (log of rms radius vs log of molecular weight where the slope of the resulting linear fit gives a degree of compactness vs elongation).

Nanoparticle tracking analysis (NTA, Malvern Nanosight) can be used to determine particle size distribution as well as particle concentration. LNP samples are diluted appropriately and injected onto a microscope slide. A camera records the scattered light as the particles are slowly infused through field of view. After the movie is captured, the Nanoparticle Tracking Analysis processes the movie by tracking pixels and calculating a diffusion coefficient. This diffusion coefficient can be translated into the hydrodynamic radius of the particle. The instrument also counts the number of individual particles counted in the analysis to give particle concentration.

Cryo-electron microscopy (“cryo-EM”) can be used to determine the particle size, morphology, and structural characteristics of an LNP.

Lipid compositional analysis of the LNPs can be determined from liquid chromatography followed by charged aerosol detection (LC-CAD). This analysis can provide a comparison of the actual lipid content versus the theoretical lipid content.

LNP compositions are analyzed for average particle size, polydispersity index (pdi), total RNA content, encapsulation efficiency of RNA, and zeta potential. LNP compositions may be further characterized by lipid analysis, AF4-MALS, NTA, and/or cryo-EM. Average particle size and polydispersity are measured by dynamic light scattering (DLS) using a Malvern Zetasizer DLS instrument. LNP samples were diluted with PBS buffer prior to being measured by DLS. Z-average diameter which is an intensity-based measurement of average particle size was reported along with number average diameter and pdi. A Malvern Zetasizer instrument is also used to measure the zeta potential of the LNP. Samples are diluted 1:17 (50 μL into 800 μL) in 0.1×PBS, pH 7.4 prior to measurement.

A fluorescence-based assay (Ribogreen®, ThermoFisher Scientific) is used to determine total RNA concentration and free RNA. Encapsulation efficiency is calculated as (Total RNA−Free RNA)/Total RNA. LNP samples are diluted appropriately with 1×TE buffer containing 0.2% Triton-X 100 to determine total RNA or 1×TE buffer to determine free RNA. Standard curves are prepared by utilizing the starting RNA solution used to make the compositions and diluted in 1×TE buffer+/−0.2% Triton-X 100. Diluted RiboGreen® dye (according to the manufacturer's instructions) is then added to each of the standards and samples and allowed to incubate for approximately 10 minutes at room temperature, in the absence of light. A SpectraMax M5 Microplate Reader (Molecular Devices) is used to read the samples with excitation, auto cutoff and emission wavelengths set to 488 nm, 515 nm, and 525 nm respectively. Total RNA and free RNA are determined from the appropriate standard curves.

Encapsulation efficiency is calculated as (Total RNA−Free RNA)/Total RNA. The same procedure may be used for determining the encapsulation efficiency of a DNA-based cargo component. For single-strand DNA Oligreen Dye may be used, and for double-strand DNA, Picogreen Dye.

AF4-MALS is used to look at molecular weight and size distributions as well as secondary statistics from those calculations. LNPs are diluted as appropriate and injected into a AF4 separation channel using an HPLC autosampler where they are focused and then eluted with an exponential gradient in cross flow across the channel. All fluid is driven by an HPLC pump and Wyatt Eclipse Instrument. Particles eluting from the AF4 channel flow through a UV detector, multi-angle light scattering detector, quasi-elastic light scattering detector and differential refractive index detector. Raw data is processed by using a Debeye model to determine molecular weight and rms radius from the detector signals.

Lipid components in LNPs are analyzed quantitatively by HPLC coupled to a charged aerosol detector (CAD). Chromatographic separation of 4 lipid components is achieved by reverse phase HPLC. CAD is a destructive mass based detector which detects all non-volatile compounds and the signal is consistent regardless of analyte structure.

Cas9 mRNA and gRNA Cargos

The Cas9 mRNA cargo was prepared by in vitro transcription. Capped and polyadenylated Cas9 mRNA comprising 1×NLS (SEQ ID NO: 3) or a sequence of Table 24 of PCT/US2019/053423 (which is hereby incorporated by reference) was generated by in vitro transcription using a linearized plasmid DNA template and T7 RNA polymerase. For example, plasmid DNA containing a T7 promoter and a 100 nt poly(A/T) region can be linearized by incubating at 37° C. for 2 hours with XbaI with the following conditions: 200 ng/μL plasmid, 2 U/μL XbaI (NEB), and 1× reaction buffer. The XbaI can be inactivated by heating the reaction at 65° C. for 20 min. The linearized plasmid can be purified from enzyme and buffer salts using a silica maxi spin column (Epoch Life Sciences) and analyzed by agarose gel to confirm linearization. The IVT reaction to generate Cas9 modified mRNA can be performed by incubating at 37° C. for 4 hours in the following conditions: 50 ng/μL linearized plasmid; 2 mM each of GTP, ATP, CTP, and N1-methyl pseudo-UTP (Trilink); 10 mM ARCA (Trilink); 5 U/μL T7 RNA polymerase (NEB); 1 U/μL Murine RNase inhibitor (NEB); 0.004 U/μL Inorganic E. coli pyrophosphatase (NEB); and 1× reaction buffer. After the 4 h incubation, TURBO DNase (ThermoFisher) was added to a final concentration of 0.01 U/μL, and the reaction was incubated for an additional 30 minutes to remove the DNA template. The Cas9 mRNA was purified with an LiCl precipitation-containing method.

The sgRNA (e.g., G650; SEQ ID NO: 2) was chemically synthesized and optionally sourced from a commercial supplier.

LNPs

These LNPs were formulated at a 1:1 w/w ratio of single guide RNA and Cas9 mRNA. Molar concentrations of lipids in the lipid component of the LNPs are expressed as mol % amine lipid/DSPC/cholesterol/PEG-2k-DMG, e.g. 50/10/38.5/1.5. The final LNPs were characterized to determine the encapsulation efficiency, polydispersity index, and average particle size according to the analytical methods provided above. Analysis of average particle size, polydispersity (PDI), total RNA content and encapsulation efficiency of RNA are shown in Table 3.

TABLE 3
Composition Analytics

| | | | | Z-Ave | | Num Ave |
|---|---|---|---|---|---|---|
| | | Conc. | Encapsulation | Size | | Size |
| Ionizable Lipid | Composition | (mg/ml) | (%) | (nm) | PDI | (nm) |
| Compound 19 | 50/9/38/3 | 1 | 98 | 82.71 | 0.056 | 64.97 |
| Compound 1 | 50/10/38.5/1.5 | 0.5 | 98 | 79.49 | 0.105 | 57.04 |
| Compound 2 | 50/10/38.5/1.5 | 0.5 | 98 | 79.89 | 0.068 | 59.46 |
| Compound 3 | 50/10/38.5/1.5 | 0.5 | 98 | 75.79 | 0.056 | 57.41 |
| Compound 5 | 50/10/38.5/1.5 | 0.5 | 98 | 83.93 | 0.099 | 59.69 |
| Compound 6 | 50/10/38.5/1.5 | 0.5 | 99 | 78.59 | 0.075 | 58.17 |

Structure and method of synthesis of Compound 19 are disclosed in US 2017/0196809A1, which is incorporated herein in its entirety.

LNPs were administered to mice by a single dose at 0.1 mg/kg, unless otherwise noted and genomic DNA was isolated for NGS analysis as described below.

LNP Delivery In Vivo

CD-1 female mice, ranging from 6-10 weeks of age were used in each study. Animals were weighed and grouped according to body weight for preparing dosing solutions based on group average weight. LNPs were dosed via the lateral tail vein in a volume of 0.2 mL per animal (approximately 10 mL per kilogram body weight). The animals were periodically observed post dose for adverse effects for at least 24 hours post dose. Animals were euthanized at 6 or 7 days by exsanguination via cardiac puncture under isoflurane anesthesia. Blood was collected into serum separator tubes or into tubes containing buffered sodium citrate for plasma as described herein. For studies involving in vivo editing, liver tissue was collected from each animal for DNA extraction and analysis.

Cohorts of mice were measured for liver editing by Next-Generation Sequencing (NGS).

NGS Sequencing

In brief, to quantitatively determine the efficiency of editing at the target location in the genome, genomic DNA was isolated and deep sequencing was utilized to identify the presence of insertions and deletions introduced by gene editing.

PCR primers were designed around the target site (e.g., B2M), and the genomic area of interest was amplified. Additional PCR was performed according to the manufacturer's protocols (Illumina) to add the necessary chemistry for sequencing. The amplicons were sequenced on an Illumina MiSeq instrument. The reads were aligned to the human reference genome (e.g., hg38) after eliminating those having low quality scores. The resulting files containing the reads were mapped to the reference genome (BAM files), where reads that overlapped the target region of interest were selected and the number of wild type reads versus the number of reads which contain an insertion, substitution, or deletion was calculated.

The editing percentage (e.g., the “editing efficiency” or “percent editing”) is defined as the total number of sequence reads with insertions or deletions over the total number of sequence reads, including wild type.

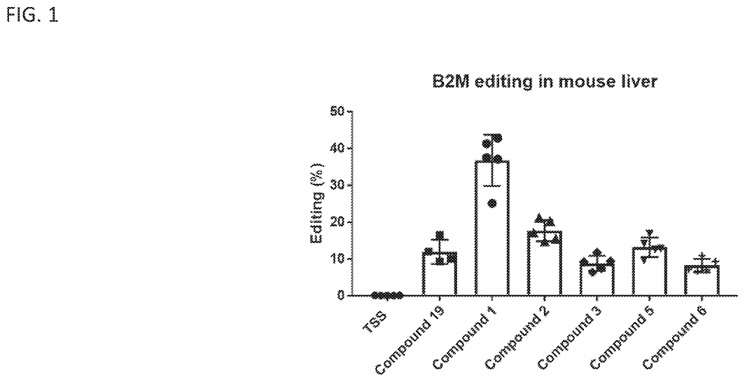

FIG. 1 shows editing percentages in mouse liver as measured by NGS. As shown in FIG. 1 and Table 4, in vivo editing percentages range from about 8% to over 35% liver editing.

TABLE 4
Editing efficiency of B2M in mouse liver

| Condition | Editing (%) | Standard Deviation | Sample number (n) |
|---|---|---|---|
| TSS | 0.0 | 0.1 | 5 |
| Compound 19 | 12.0 | 3.2 | 4 |
| Compound 1 | 36.8 | 7.0 | 5 |
| Compound 2 | 17.7 | 2.9 | 5 |
| Compound 3 | 8.8 | 2.0 | 5 |
| Compound 5 | 13.2 | 2.7 | 5 |
| Compound 6 | 8.2 | 1.8 | 5 |

### Example 53—Dose Response of Editing in Liver

To assess the scalability of dosing, a dose response experiment was performed in vivo with compound 1. Cas9 mRNA of Example 52 was formulated as LNPs with a guide RNA targeting either TTR (G282; SEQ ID NO: 1) or B2M (G650; SEQ ID NO: 2). These LNPs were formulated at a 1:1 w/w ratio of a single guide RNA and Cas9 mRNA. The LNPs were assembled using the cross flow procedure with compositions as described in Table 5. All LNPs had an N:P ratio of 6.0 and were used at the concentration described in Table 5 after concentration using Amicon PD-10 filters (GE Healthcare), if necessary.

LNP compositions were analyzed for average particle size, polydispersity (pdi), total RNA content and encapsulation efficiency of RNA as described in Example 52.

Analysis of average particle size, polydispersity (PDI), total RNA content and encapsulation efficiency of RNA are shown in Table 5.

TABLE 5
Composition Analytics

| | | | | | Z-Ave | | Num Ave |
|---|---|---|---|---|---|---|---|
| | | Encapsulation | | Concentration | Size | | Size |
| Ionizable Lipid | Composition | (%) | gRNA | (mg/ml) | (nm) | PDI | (nm) |
| Compound 19 | 50/9/38/3 | 99 | G282 | 0.05 | 79.83 | 0.015 | 62.86 |
| Compound 19 | 50/9/38/3 | 98 | G650 | 1 | 82.71 | 0.056 | 64.97 |
| Compound 1 | 50/10/38.5/1.5 | 97 | G282 | 0.043 | 75.77 | 0.008 | 61.19 |
| Compound 1 | 50/10/38.5/1.5 | 98 | G650 | 0.593 | 80.96 | 0.028 | 65.11 |

CD-1 female mice were dosed i.v. at 0.1 mpk or 0.3 mpk. At 6 days post-dose, animals were sacrificed. For animals dosed with G282 targeting TTR, blood and the liver were collected and serum TTR and editing were measured. For animals dosed with G650 targeting B2M, liver was collected and editing was measured.

Transthyretin (TTR) ELISA Analysis

Blood was collected and the serum was isolated as indicated. The total mouse TTR serum levels were determined using a Mouse Prealbumin (Transthyretin) ELISA Kit (Aviva Systems Biology, Cat. OKIA00111). Briefly, sera were serial diluted with kit sample diluent to a final dilution of 10,000-fold for 0.1 mpk dose and 2,500-fold for 0.3 mpk. This diluted sample was then added to the ELISA plates and the assay was then carried out according to directions.

Table 6 and FIG. 2A-FIG. 2C show TTR editing in liver and serum TTR levels results. Compound 1 formulations showed higher TTR editing in the liver than Compound 19 formulations at each dose. The Compound 1 formulation showed editing of TTR in the 55-60% range with both the 0.1 mpk and 0.3 mpk doses, indicating efficacy at low doses.

TABLE 6
TTR liver editing and serum TTR levels for dose response

| | | | TTR Editing | | Serum TTR | | % TSS | Sample |
|---|---|---|---|---|---|---|---|---|
| | Dose | TTR Editing | Standard | Serum TTR | Standard | | Standard | Number |
| Condition | (mpk) | (%) | Deviation | (kg/ml) | Dev | TSS | Deviation | (n) |
| TSS | | 0.1 | 0.1 | 71 | 0 | 00 | 2 | 5 |
| Compound 19 | 0.1 | 13.8 | 3.9 | 55 | 28 | 85 | 7 | 5 |
| Compound 19 | 0.3 | 45.9 | 7.8 | 55 | 103 | 3 | 3 | 4 |
| Compound 1 | 0.1 | 55.4 | 4.8 | 19 | 82 | 5 | 1 | 5 |
| Compound 1 | 0.3 | 59.3 | 6.5 | 5 | 8 | 5 | 2 | 5 |

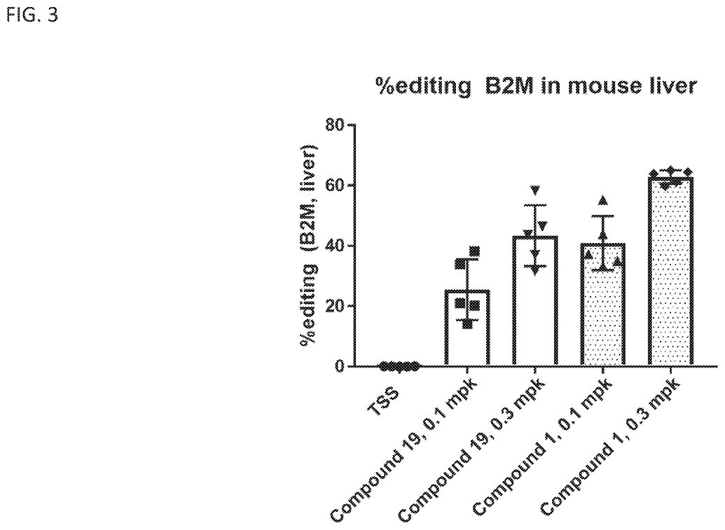

Table 7 and FIG. 3 show B2M editing results in liver. Compound 1 showed higher B2M editing in the liver than Compound 19 at each dose. Compound 1 and Compound 19 increased editing of B2M in liver significantly between the 0.1 mpk and 0.3 mpk doses.

TABLE 7
B2M liver editing for dose response

| | | | | Sample |
|---|---|---|---|---|
| | Dose | Editing | Standard | Number |
| Condition | (mpk) | (%) | Deviation | (n) |
| TSS | | 0.1 | 0.1 | 5 |
| Compound 19 | 0.1 | 25.5 | 10.1 | 5 |
| Compound 19 | 0.3 | 43.4 | 10.1 | 5 |
| Compound 1 | 0.1 | 41.0 | 9.0 | 5 |
| Compound 1 | 0.3 | 62.9 | 2.3 | 5 |

### Example 54—B2M Editing in Mouse Liver with Compositions Comprising Compound 4

Editing was assessed with different doses and PEG lipid concentrations in compositions comprising compound 4. The Cas9 mRNA described in Example 52 was formulated as LNPs with a guide RNA targeting B2M (G650; SEQ ID NO: 2). These LNPs were formulated at a 1:1 w/w ratio of single guide RNA and Cas9 mRNA. The LNPs were assembled using the cross flow procedure with compositions as described in Table 8. All LNPs had an N:P ratio of 6.0. All LNPs were concentrated using Amicon PD-10 filters (GE Healthcare) and/or tangential flow filtration, and were used at the concentration described in Table 8.

LNP compositions were analyzed for average particle size, polydispersity (pdi), total RNA content and encapsulation efficiency of RNA as described in Example 52.

Analysis of average particle size, polydispersity (PDI), total RNA content and encapsulation efficiency of RNA are shown in Table 8.

TABLE 8
Composition Analytics

| | | Conc. | Encapsulation | Z-Ave | | Num Ave |
|---|---|---|---|---|---|---|
| Ionizable Lipid | Composition | (mg/ml) | (%) | Size (nm) | PDI | Size (nm) |
| Compound 19 | 50/9/38/3 | 1 | 98 | 82.71 | 0.056 | 64.97 |
| Compound 4 | 50/10/38.5/1.5 | 0.5 | 97 | 77.33 | 0.056 | 58.43 |

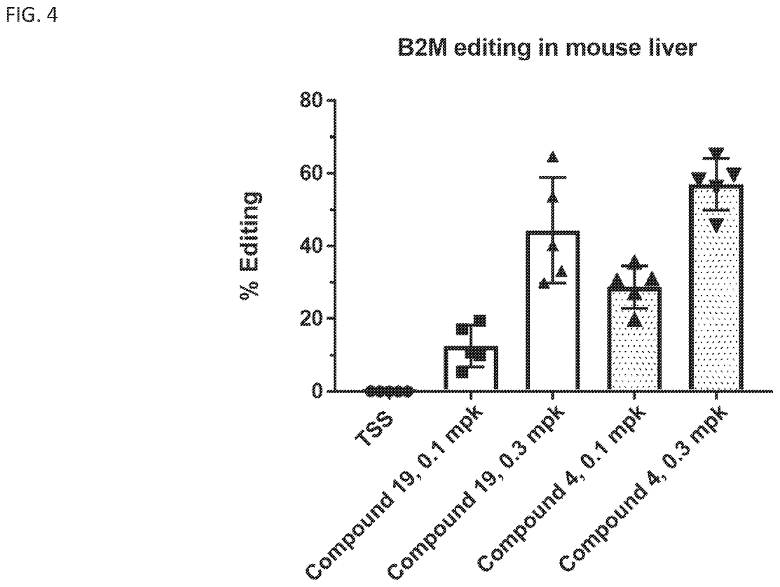

CD-1 female mice were dosed i.v. at 0.1 mpk or 0.3 mpk. At 7 days post-dose, animals were sacrificed, liver was collected and editing was measured by NGS. Table 9 and FIG. 4 show B2M editing results in liver. The compositions comprising Compound 4 showed increased editing at the 0.3 mpk dose compared to the 0.1 mpk dose, as did the Compound 19 comparison composition.

TABLE 9
B2M editing in mouse liver using Compound 4

| | | | | Sample |
|---|---|---|---|---|
| | Dose | % | Standard | Number |
| Condition | (mpk) | Editing | Deviation | (n) |
| TSS | — | 0 | 0 | 5 |
| Compound 19 | 0.1 | 13 | 6 | 5 |
| Compound 19 | 0.3 | 44 | 15 | 5 |
| Compound 4 | 0.1 | 29 | 6 | 5 |
| Compound 4 | 0.3 | 57 | 7 | 5 |

### Example 55—TTR Editing in Mouse Liver

Editing was assessed for additional compositions. The Cas9 mRNA described in Example 52 was formulated as LNPs with a guide RNA targeting TTR (G282; SEQ ID NO: 1). These LNPs were formulated at a 1:1 w/w ratio of single guide RNA and Cas9 mRNA. The LNPs were assembled using the cross flow procedure as described in Example 52 with compositions as described in Table 10. All LNPs had an N:P ratio of 6.0. LNPs were used at the concentration described in Table 10. LNP compositions were analyzed for average particle size, polydispersity (pdi), total RNA content and encapsulation efficiency of RNA as described in Example 52.

Analysis of average particle size, polydispersity (PDI), total RNA content and encapsulation efficiency of RNA are shown in Table 10.

TABLE 10
Composition Analytics

| | | Conc. | Encapsulation | Z-Ave | | Num Ave |
|---|---|---|---|---|---|---|
| Ionizable lipid | Composition | (mg/ml) | (%) | Size (nm) | PDI | Size (nm) |
| Compound 19 | 50/9/38/3 | 0.062 | 98 | 81.76 | 0.025 | 64.95 |
| Compound 1 | 50/10/38.5/1.5 | 0.057 | 97 | 69.94 | 0.05 | 55.38 |
| Compound 7 | 50/10/38.5/1.5 | 0.065 | 92 | 74.45 | 0.065 | 55.7 |
| Compound 8 | 50/10/38.5/1.5 | 0.058 | 84 | 87.86 | 0.085 | 60.2 |
| Compound 10 | 50/10/38.5/1.5 | 0.069 | 95 | 72.36 | 0.049 | 55.72 |

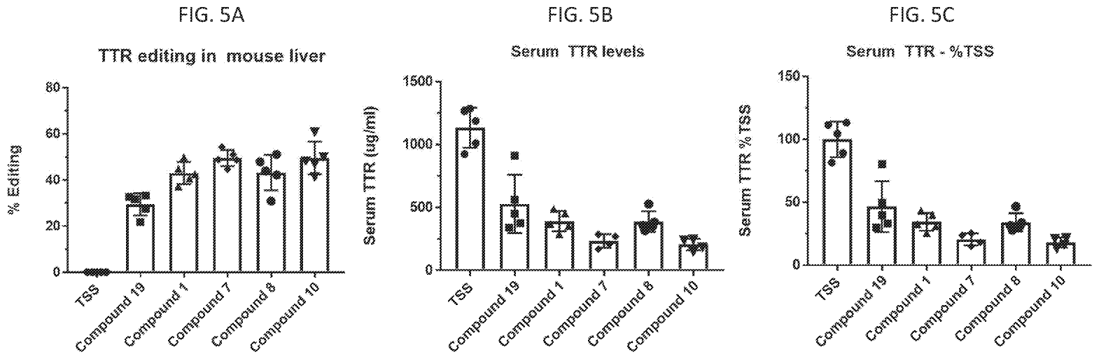

CD-1 female mice were dosed i.v. at 0.1 mpk. At 7 days post-dose, animals were sacrificed. Blood and the liver were collected and serum TTR and editing were measured as described above. Table 11 and FIG. 5 show TTR editing in liver and serum TTR levels results.

TABLE 11

| | | Editing | Sample | | | | | Sample |
|---|---|---|---|---|---|---|---|---|
| | % | Standard | Number | Serum TTR | Serum TTR | Serum TTR | % TSS | Number |
| Condition | Editing | Deviation | (n) | (μg/ml) | SD | (% TSS) | SD | (n) |
| TSS | 0 | 0 | 5 | 1136 | 159 | 100 | 14 | 5 |
| Compound 19 | 29 | 5 | 5 | 528 | 231 | 46 | 20 | 5 |
| Compound 1 | 43 | 5 | 5 | 391 | 80 | 34 | 7 | 5 |
| Compound 7 | 50 | 4 | 5 | 234 | 54 | 21 | 5 | 4 |
| Compound 8 | 43 | 8 | 5 | 387 | 83 | 34 | 7 | 5 |
| Compound 10 | 50 | 7 | 5 | 206 | 46 | 18 | 4 | 4 |

Each amine lipid of Formula(J) or Formula (II) tested in this example showed ˜40-50% editing of TTR with corresponding decreases of ˜80%0 of serum TTR levels. These LNPs compared favorably to the reference.

### Example 56—TTR Editing in Mouse Liver

Editing was assessed for additional amine lipid formulations. The Cas9 mRNA of Example 52 was formulated as LNPs with a guide RNA targeting TTR (G282; SEQ ID NO: 1). The LNPs were assembled using the cross flow procedure as described in Example 52 with compositions as described in Table 12. All LNPs had an N ratio of 6.0. LNPs were used at the concentration of about 0.06 mg/ml. LNP formulations were analyzed for average particle size, polydispersity (pdi), total RNA content and encapsulation efficiency of RNA as described in Example 52. Analysis of average particle size, polydispersity (PDI), total RNA content and encapsulation efficiency of RNA are shown in Table 12.

TABLE 12
Formulation Analytics

| | | Encap- | Z-Ave | | Num Ave |
|---|---|---|---|---|---|
| Ionizable | Composition | sulation | Size | | Size |
| Lipid | ratio | (%) | (nm) | PDI | (nm) |
| Compound 19 | 50/9/38/3 | 97 | 79.18 | 0.047 | 63.19 |
| Compound 18 | 50/10/38.5/1.5 | 81 | 106.6 | 0.112 | 69.77 |
| Compound 5 | 50/10/38.5/1.5 | 98 | 108.1 | 0.273 | 48.59 |

CD-1 female mice were dosed i.v. at 0.1 mpk. At 7 days post-dose, animals were sacrificed. Blood and the liver were collected and serum TTR and editing were measured as described above. Table 13 describes the TTR editing in liver and serum TTR levels results.

TABLE 13
Editing in mouse liver and serum TTR levels

| | | | | | | | Sample |
|---|---|---|---|---|---|---|---|
| | Editing | Editing | Serum TTR | TTR | Serum TTR | % TSS | number |
| Condition | % | SD | μg/ml | SD | (% TSS) | SD | (n) |
| TSS | 0 | 0 | 801 | 115 | 100 | 14 | 5 |
| Compound 19 | 15 | 9 | 865 | 197 | 108 | 25 | 5 |
| Compound 18 | 33 | 8 | 415 | 95 | 52 | 12 | 5 |
| Compound 5 | 9 | 3 | 738 | 122 | 92 | 15 | 5 |

### Example 57—Measurement of Expressed Protein

With mRNA cargo, protein expression is one measure of delivery by a lipid nanoparticle. For example, ELISA can be used to measure protein levels in biological samples for a wide variety of proteins. The following protocol can be used to measure an expressed protein, e.g. Cas9 protein expression, from biological samples. Briefly, total protein concentration of cleared cell lysate is determined by bicinchoninic acid assay. An MSD GOLD 96-well Streptavidin SECTOR Plate (Meso Scale Diagnostics, Cat. L15SA-1) is prepared according to manufacturer's protocol using Cas9 mouse antibody (Origene, Cat. CF811179) as the capture antibody and Cas9 (7A9-3A3) Mouse mAb (Cell Signaling Technology, Cat. 14697) as the detection antibody. Recombinant Cas9 protein is used as a calibration standard in Diluent 39 (Meso Scale Diagnostics) with 1× Halt™ Protease Inhibitor Cocktail, EDTA-Free (ThermoFisher, Cat. 78437). ELISA plates are read using the Meso Quickplex SQ120 instrument (Meso Scale Discovery) and data is analyzed with Discovery Workbench 4.0 software package (Meso Scale Discovery).

### Example 58—TTR Editing in Mouse Liver

Editing was assessed for additional compositions. The Cas9 mRNA described in Example 52 was formulated as LNPs with a guide RNA targeting TTR (G282; SEQ ID NO: 1). These LNPs were formulated at a 1:1 w/w ratio of single guide RNA and Cas9 mRNA. The LNPs were assembled using the cross flow procedure as described in Example 52 with compositions as described in Table 14. All LNPs had an N:P ratio of 6.0. LNPs were used at the concentration described in Table 14. LNP compositions were analyzed for average particle size, polydispersity (pdi), total RNA content and encapsulation efficiency of RNA as described in Example 52. Analysis of average particle size, polydispersity (PDI), total RNA content and encapsulation efficiency of RNA are shown in Table 14.

TABLE 14
Composition Analytics

| | | Conc. | Encapsulation | Z-Ave | | Num Ave |
|---|---|---|---|---|---|---|
| Compound | Composition | (mg/ml) | (%) | Size (nm) | PDI | Size (nm) |
| Compound 19 | 50/9/38/3 | 0.05 | 99 | 84.99 | 0.007 | 71.1 |
| Compound 1 | 50/10/38.5/1.5 | 0.057 | 97 | 69.94 | 0.05 | 55.38 |
| Compound 11 | 50/10/38.5/1.5 | 0.074 | 86 | 89.3 | 0.137 | 54.58 |
| Compound 12 | 50/10/38.5/1.5 | 0.073 | 98 | 75.44 | 0.014 | 62.44 |
| Compound 13 | 50/10/38.5/1.5 | 0.07 | 98 | 77.34 | 0.04 | 63.34 |
| Compound 14 | 50/10/38.5/1.5 | 0.078 | 98 | 82.2 | 0.039 | 65.34 |
| Compound 15 | 50/10/38.5/1.5 | 0.081 | 88 | 82.93 | 0.092 | 57.97 |
| Compound 16 | 50/10/38.5/1.5 | 0.054 | 78 | 109.5 | 0.132 | 66.66 |
| Compound 17 | 50/10/38.5/1.5 | 0.071 | 91 | 68.72 | 0.056 | 54.25 |

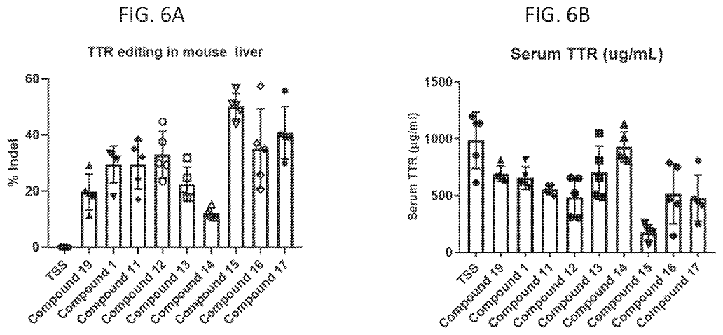

Five CD-1 female mice were dosed i.v. at 0.1 mpk for each condition. At 6 days post-dose, animals were sacrificed. Blood and the liver were collected and serum TTR and editing were measured as described above. Table 15 and FIG. 6 show TTR editing in liver and serum TTR levels results.

TABLE 15
Editing in mouse liver and serum TTR levels

| | Editing | | Serum TTR | | | |
|---|---|---|---|---|---|---|
| | Mean % | | Serum TTR | | % | |
| Compound | Indel | SD | (ug/ml) | SD | TSS | N |
| TSS | 0.1 | 0.0 | 989 | 248 | 100% | 5 |
| Compound 19 | 29.7 | 7.1 | 581 | 180 | 59% | 5 |
| Compound 19 | 19.7 | 6.4 | 695 | 69 | 70% | 5 |
| Compound 1 | 29.5 | 6.5 | 656 | 98 | 66% | 5 |
| Compound 11 | 29.4 | 8.6 | 553 | 41 | 56% | 5 |
| Compound 12 | 33.0 | 8.3 | 490 | 176 | 50% | 5 |
| Compound 13 | 22.6 | 6.0 | 703 | 233 | 71% | 5 |
| Compound 14 | 12.1 | 1.9 | 928 | 134 | 94% | 5 |
| Compound 15 | 50.3 | 4.6 | 179 | 68 | 18% | 5 |
| Compound 16 | 35.2 | 14.1 | 516 | 264 | 52% | 5 |
| Compound 17 | 40.8 | 9.4 | 479 | 204 | 48% | 5 |

### Example 59—TTR Editing in Mouse Liver

Editing was assessed for additional compositions. The Cas9 mRNA described in Example 52 was formulated as LNPs with a guide RNA targeting TTR (G282; SEQ ID NO: 1). These LNPs were formulated at a 1:1 w/w ratio of single guide RNA and Cas9 mRNA. The LNPs were assembled using the cross flow procedure as described in Example 52 with compositions as described in Table 16. All LNPs had an N:P ratio of 6.0. LNPs were used at the concentration of about 0.05 mg/ml. LNP compositions were analyzed for average particle size, polydispersity (pdi), total RNA content and encapsulation efficiency of RNA as described in Example 52. Analysis of average particle size, polydispersity (PDI), total RNA content and encapsulation efficiency of RNA are shown in Table 16.

TABLE 16
Composition Analytics

| | | Encap- | Z-Ave | | Num Ave |
|---|---|---|---|---|---|
| | | sulation | Size | | Size |
| Compound | Composition | (%) | (nm) | PDI | (nm) |
| Compound 19 | 50/9/38/3 | 98 | 86.84 | 0.02 | 71.49 |
| Compound 18 | 50/10/38.5/1.5 | 98 | 75.79 | 0.093 | 53.06 |
| Compound 1 | 50/10/38.5/1.5 | 96 | 72.21 | 0.04 | 57.78 |
| Compound 10 | 50/10/38.5/1.5 | 98 | 73.31 | 0.044 | 57.14 |
| Compound 20 | 50/10/38.5/1.5 | 83 | 84.49 | 0.102 | 58.65 |

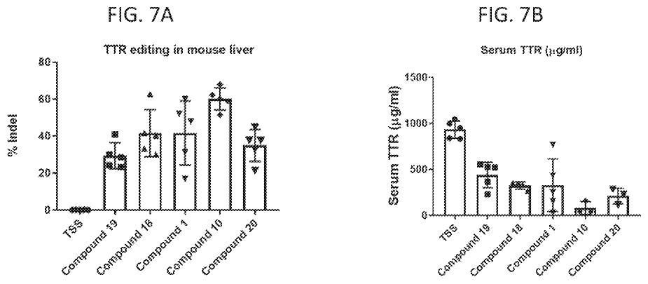

CD-1 female mice were dosed i.v. at 0.1 mpk. At 7 days post-dose, animals were sacrificed. Blood and the liver were collected and serum TTR and editing were measured as described above. Table 17 and FIG. 7 show TTR editing in liver and serum TTR levels results.

TABLE 17
Editing in mouse liver and serum TTR levels

| | Editing | | | Serum TTR | | | |
|---|---|---|---|---|---|---|---|
| | Mean % | | | Serum TTR | | % | |
| Compound | Indel | SD | N | (ug/ml) | SD | TSS | N |
| TSS | 0.1 | 0.0 | 5 | 933 | 95 | 100% | 5 |
| Compound 19 | 29.3 | 7.1 | 5 | 438 | 139 | 47% | 5 |
| Compound 18 | 41.6 | 12.8 | 5 | 324 | 39 | 35% | 4 |
| Compound 1 | 41.6 | 17.4 | 5 | 327 | 287 | 35% | 5 |
| Compound 10 | 60.1 | 5.9 | 5 | 80 | 70 | 9% | 3 |
| Compound 20 | 35.0 | 8.7 | 5 | 210 | 85 | 23% | 3 |

### Example 60—TTR Editing in Mouse Liver

Editing was assessed for additional compositions. The Cas9 mRNA described in Example 52 was formulated as LNPs with a guide RNA targeting TTR (G502; SEQ ID NO: 4). These LNPs were formulated at a 1:2 w/w ratio of single guide RNA and Cas9 mRNA. The LNPs were assembled using the cross flow procedure as described in Example 52 with compositions as described in Table 18. All LNPs had an N:P ratio of 6.0. LNPs were used at the concentration of about 0.05. LNP compositions were analyzed for average particle size, polydispersity (pdi), total RNA content and encapsulation efficiency of RNA as described in Example 52. Analysis of average particle size, polydispersity (PDI), total RNA content and encapsulation efficiency of RNA are shown in Table 18.

TABLE 18
Composition Analytics

| | | Encap- | Z-Ave | | Num Ave |
|---|---|---|---|---|---|
| | | sulation | Size | | Size |
| Compound | Composition | (%) | (nm) | PDI | (nm) |
| Compound 19 | 50/9/38/3 | 99 | 88.6 | 0.033 | 73.15 |
| Compound 1 | 50/10/38.5/1.5 | 97 | 74.5 | 0.037 | 58.01 |
| Compound 22 | 50/10/38.5/1.5 | 98 | 82.91 | 0.029 | 66.68 |
| Compound 23 | 50/10/38.5/1.5 | 93 | 79.04 | 0.054 | 61.86 |
| Compound 25 | 50/10/38.5/1.5 | 92 | 66.83 | 0.065 | 50.74 |

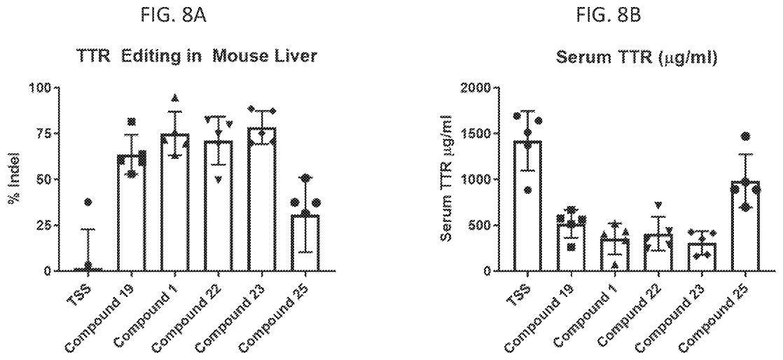

CD-1 female mice were dosed i.v. at 0.1 mpk. At 6 days post-dose, animals were sacrificed. Blood and the liver were collected and serum TTR and editing were measured as described above. Table 19 and FIG. 8 show TTR editing in liver and serum TTR levels results.

TABLE 19
Editing in mouse liver and serum TTR levels

| | Editing | | Serum TTR | | | |
|---|---|---|---|---|---|---|
| | Mean % | | Serum TTR | | % | |
| Compound | Indel | SD | (ug/ml) | SD | TSS | N |
| TSS | 0.2 | 0.3 | 1422 | 325 | 100% | 5 |
| Compound 19 | 41.4 | 5.5 | 517 | 153 | 36% | 5 |
| Compound 1 | 46.3 | 13.0 | 353 | 170 | 25% | 5 |
| Compound 22 | 45.6 | 11.8 | 410 | 186 | 29% | 5 |
| Compound 23 | 53.6 | 8.4 | 307 | 128 | 22% | 5 |
| Compound 25 | 15.3 | 11.2 | 985 | 290 | 69% | 5 |

### Example 61—TTR Editing in Mouse Liver

Editing was assessed for additional compositions. The Cas9 mRNA described in Example 52 was formulated as LNPs with a guide RNA targeting TTR (G282; SEQ ID NO: 1). These LNPs were formulated at a 1:1 w/w ratio of single guide RNA and Cas9 mRNA. The LNPs were assembled using the cross flow procedure as described in Example 52 with compositions as described in Table 20. All LNPs had an N:P ratio of 6.0. LNPs were used at the concentration as described in Table 20. LNP compositions were analyzed for average particle size, polydispersity (pdi), total RNA content and encapsulation efficiency of RNA as described in Example 52. Analysis of average particle size, polydispersity (PDI), total RNA content and encapsulation efficiency of RNA are shown in Table 20.

TABLE 20
Composition Analytics

| | | Conc. | Encapsulation | Z-Ave | | Num Ave |
|---|---|---|---|---|---|---|
| Compound | Composition | (mg/ml) | (%) | Size (nm) | PDI | Size (nm) |
| Compound 19 | 50/9/38/3 | 0.05 | 98 | 86.84 | 0.02 | 71.49 |
| Compound 18 | 50/10/38.5/1.5 | 0.04 | 90 | 74.67 | 0.083 | 52.25 |
| Compound 4 | 50/10/38.5/1.5 | 0.05 | 97 | 87.27 | 0.051 | 68.75 |
| Compound 9 | 50/10/38.5/1.5 | 0.05 | 97 | 75.32 | 0.021 | 60.79 |
| Compound 10 | 50/10/38.5/1.5 | 0.05 | 94 | 76.35 | 0.059 | 57.86 |
| Compound 26 | 50/10/38.5/1.5 | 0.05 | 99 | 63.25 | 0.07 | 48.37 |

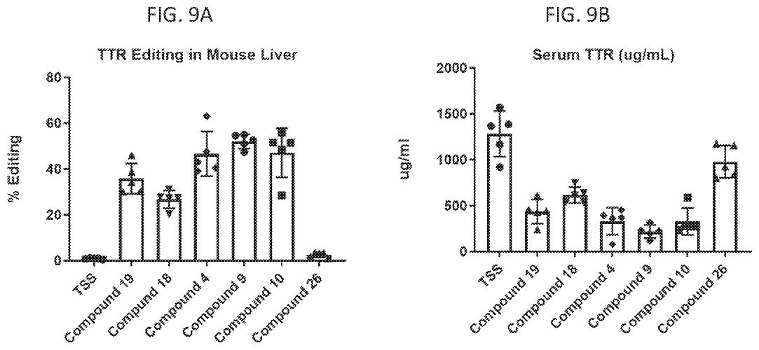

CD-1 female mice were dosed i.v. at 0.1 mpk. At 7 days post-dose, animals were sacrificed. Blood and the liver were collected and serum TTR and editing were measured as described above. Table 21 and FIG. 9 show TTR editing in liver and serum TTR levels results.

TABLE 21
Editing in mouse liver and serum TTR levels

| | Editing | | Serum TTR | | | |
|---|---|---|---|---|---|---|
| | Mean % | | Serum TTR | | % | |
| Compound | Indel | SD | (ug/ml) | SD | TSS | N |
| TSS | 0.9 | 0.3 | 1282 | 248 | 100% | 5 |
| Compound 19 | 35.9 | 6.6 | 438 | 132 | 34% | 5 |
| Compound 18 | 26.8 | 3.9 | 615 | 87 | 48% | 5 |
| Compound 4 | 46.7 | 9.7 | 333 | 146 | 26% | 5 |
| Compound 9 | 52.1 | 3.1 | 218 | 72 | 17% | 5 |
| Compound 10 | 47.2 | 10.8 | 330 | 146 | 26% | 5 |
| Compound 26 | 2.6 | 1.1 | 979 | 177 | 76% | 5 |

### Example 62—Dose Response of Editing in Liver

To assess the scalability of dosing, a dose response experiment was performed in vivo. Cas9 mRNA of Example 52 was formulated as LNPs with a guide RNA targeting either TTR (G282; SEQ ID NO: 1). These LNPs were formulated at a 1:2 w/w ratio of single guide RNA and Cas9 mRNA. The LNPs were assembled using the cross flow procedure with compositions as described in Table 22. All LNPs had an N:P ratio of 6.0 and were used at the concentration described in Table 22 after concentration using Amicon PD-10 filters (GE Healthcare), if necessary.

LNP compositions were analyzed for average particle size, polydispersity (pdi), total RNA content and encapsulation efficiency of RNA as described in Example 52. Analysis of average particle size, polydispersity (PDI), total RNA content and encapsulation efficiency of RNA are shown in Table 22.

TABLE 22
Composition Analytics

| | | Encap- | Z-Ave | | Num Ave |
|---|---|---|---|---|---|
| | | sulation | Size | | Size |
| Compound | Composition | (%) | (nm) | PDI | (nm) |
| Compound 19 | 50/9/38/3 | 99 | 88.6 | 0.033 | 73.15 |
| Compound 1 | 50/10/38.5/1.5 | 97 | 74.5 | 0.037 | 58.01 |
| Compound 10 | 50/10/38.5/1.5 | 92 | 79.49 | 0.072 | 59.35 |
| Compound 15 | 50/10/38.5/1.5 | 89 | 90.22 | 0.051 | 69.95 |
| Compound 17 | 50/10/38.5/1.5 | 94 | 62.89 | 0.072 | 44.93 |

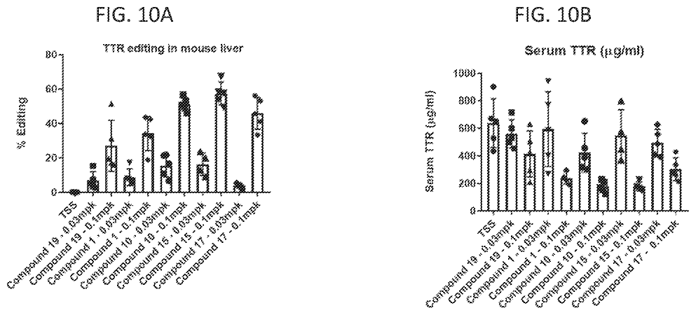

CD-1 female mice were dosed i.v. at 0.1 mpk or 0.03 mpk. At 7 days post-dose, animals were sacrificed. Blood and the liver were collected and serum TTR and editing were measured. Table 23 and FIG. 10 show TTR editing in liver and serum TTR levels results.

TABLE 23
TTR liver editing and serum TTR levels for dose response

| | | Editing | | | Serum TTR | | | |
|---|---|---|---|---|---|---|---|---|
| | Dose | Mean % | | | Serum TTR | | % | |
| Compound | (mpk) | Indel | SD | N | (ug/ml) | SD | TSS | N |
| TSS | TSS | 0.1 | 0.0 | 5 | 638 | 176 | 100% | 5 |
| Compound 19 | 0.03 | 7.0 | 5.0 | 5 | 560 | 102 | 88% | 5 |
| Compound 19 | 0.1 | 27.1 | 14.9 | 5 | 414 | 167 | 65% | 5 |
| Compound 1 | 0.03 | 8.9 | 4.8 | 5 | 594 | 271 | 93% | 5 |
| Compound 1 | 0.1 | 34.2 | 9.9 | 5 | 241 | 45 | 38% | 4 |
| Compound 10 | 0.03 | 15.6 | 6.6 | 5 | 424 | 142 | 67% | 5 |
| Compound 10 | 0.1 | 51.3 | 3.9 | 5 | 179 | 42 | 28% | 5 |
| Compound 15 | 0.03 | 16.4 | 6.6 | 4 | 548 | 188 | 86% | 4 |
| Compound 15 | 0.1 | 57.4 | 6.6 | 5 | 180 | 33 | 28% | 4 |
| Compound 17 | 0.03 | 4.0 | 1.4 | 5 | 495 | 98 | 78% | 5 |
| Compound 17 | 0.1 | 45.9 | 9.2 | 5 | 304 | 82 | 48% | 5 |

### Example 63—TTR Editing in Mouse Liver

Editing was assessed for additional compositions. The Cas9 mRNA described in Example 52 was formulated as LNPs with a guide RNA targeting TTR (G282; SEQ ID NO: 1). These LNPs were formulated at a 1:1 w/w ratio of single guide RNA and Cas9 mRNA. The LNPs were assembled using the cross flow procedure as described in Example 52 with compositions as described in Table 24. All LNPs had an N2 ratio of 6.0. LNPs were used at the concentration as described in Table 24. LNP compositions were analyzed for average particle size, polydispersity (PDI), total RNA content and encapsulation efficiency of RNA as described in Example 52. Analysis of average particle size, polydispersity (PDI), total RNA content, and encapsulation efficiency of RNA are shown in Table 24.

TABLE 24
Composition Analytics

| | | Conc. | Encapsulation | Z-Ave | | Num Ave |
|---|---|---|---|---|---|---|
| Compound | Composition | (mg/ml) | (%) | Size (nm) | PDI | Size (nm) |
| Compound 19 | 50/9/38/3 | 0.05 | 98% | 82 | 0.03 | 66 |
| Compound 1 | 50/10/38.5/1.5 | 0.06 | 98% | 70 | 0.06 | 52 |
| Compound 27 | 50/10/38.5/1.5 | 0.06 | 94% | 80 | 0.13 | 54 |
| Compound 28 | 50/10/38.5/1.5 | 0.06 | 97% | 78 | 0.30 | 42 |
| Compound 29 | 50/10/38.5/1.5 | 0.06 | 91% | 118 | 0.17 | 68 |
| Compound 30 | 50/10/38.5/1.5 | 0.06 | 93% | 108 | 0.16 | 61 |

CD-1 female mice were dosed i.v. at 0.1 mpk. At 7 days post-dose, animals were taken down. Blood and liver were collected and serum TTR and editing were measured as described above. Table 25 shows TTR editing in liver and serum TTR levels results.

TABLE 25
Editing in Mouse Liver and Serum TTR Levels

| | Editing | | | Serum TTR | | | |
|---|---|---|---|---|---|---|---|
| | Dose | Mean % | | Serum TTR | | % | |
| Compound | (mpk) | Indel | SD | (ug/ml) | SD | TSS | N |
| TSS | n/a | 0.06 | 0.05 | 807.03 | 161.51 | 100.00 | 5 |
| Compound 19 | 0.03 | 30.18 | 7.90 | 593.01 | 268.33 | 73.48 | 5 |
| | 0.1 | 56.02 | 6.27 | 134.54 | 61.46 | 16.67 | 5 |
| Compound 1 | 0.03 | 10.86 | 1.36 | 741.05 | 125.46 | 91.82 | 5 |
| | 0.1 | 41.10 | 14.39 | 351.76 | 126.24 | 43.59 | 5 |
| Compound 27 | 0.03 | 27.86 | 3.69 | 497.99 | 115.29 | 61.71 | 5 |
| | 0.1 | 57.10 | 1.99 | 197.22 | 49.71 | 24.44 | 5 |
| Compound 28 | 0.03 | 20.74 | 3.72 | 493.60 | 57.20 | 61.16 | 5 |
| | 0.1 | 42.36 | 4.36 | 321.26 | 58.34 | 39.81 | 5 |
| Compound 29 | 0.03 | 5.76 | 2.94 | 718.48 | 57.40 | 89.03 | 5 |
| | 0.1 | 15.96 | 3.94 | 660.46 | 142.13 | 81.84 | 5 |
| Compound 30 | 0.03 | 27.64 | 3.50 | 514.23 | 34.52 | 63.72 | 5 |
| | 0.1 | 62.48 | 5.87 | 125.89 | 61.45 | 15.60 | 5 |

### Example 64—TTR Editing in Mouse Liver

Editing was assessed for additional compositions. The Cas9 mRNA described in Example 52 was formulated as LNPs with a guide RNA targeting TTR (G282; SEQ ID NO: 1). These LNPs were formulated at a 1:1 w/w ratio of single guide RNA and Cas9 mRNA. The LNPs were assembled using the cross flow procedure as described in Example 52 with compositions as described in Table 26. All LNPs had an N:P ratio of 6.0. LNPs were used at the concentration as described in Table 26. LNP compositions were analyzed for average particle size, polydispersity (PDI), total RNA content and encapsulation efficiency of RNA as described in Example 52. Analysis of average particle size, polydispersity (PDI), total RNA content, and encapsulation efficiency of RNA are shown in Table 26.

TABLE 26
Composition Analytics

| | | Conc. | Encapsulation | Z-Ave | | Num Ave |
|---|---|---|---|---|---|---|
| Compound | Composition | (mg/ml) | (%) | Size (nm) | PDI | Size (nm) |
| Compound 19 | 50/9/38/3 | 1.48 | 98% | 83 | 0.03 | 65 |
| Compound 10 | 50/10/38.5/1.5 | 0.06 | 92% | 77 | 0.05 | 58 |
| Compound 42 | 50/10/38.5/1.5 | 0.06 | 97% | 122 | 0.05 | 99 |
| Compound 41 | 50/10/38.5/1.5 | 0.06 | 95% | 92 | 0.05 | 70 |
| Compound 44 | 50/10/38.5/1.5 | 0.06 | 59% | 185 | 0.25 | 92 |
| Compound 43 | 50/10/38.5/1.5 | 0.06 | 94% | 74 | 0.05 | 56 |
| Compound 46 | 50/10/38.5/1.5 | 0.06 | 98% | 104 | 0.03 | 86 |
| Compound 40 | 50/10/38.5/1.5 | 0.06 | 96% | 83 | 0.06 | 64 |
| | 50/10/38.5/1.5 | 0.06 | 100% | 57 | 0.06 | 43 |
| | 50/10/38.5/1.5 | 0.06 | 90% | 88 | 0.05 | 69 |

CD-1 female mice were dosed i.v. at 0.1 mpk. At 7 days post-dose, animals were taken down. Blood and liver were collected and serum TTR and editing were measured as described above. Table 27 shows TTR editing in liver and Serum TTR levels results.

TABLE 27
Editing in Mouse Liver and Serum TTR Levels

| | Editing | | | Serum TTR | | | |
|---|---|---|---|---|---|---|---|
| | | Mean | | Serum | | Serum | |
| | Dose | % | | TTR | | TTR | |
| Compound | (mpk) | Indel | SD | (ug/ml) | SD | (% KD) | N |
| TSS | n/a | 0.2 | 0.05 | 572.6 | 13.01 | | 5 |
| Compound 19 | 0.03 | 5.2 | 1.28 | 622.0 | 8.11 | −8.6 | 5 |
| Compound 10 | 0.03 | 27.2 | 5.15 | 409.2 | 22.35 | 28.5 | 5 |
| Compound 42 | 0.03 | 9.6 | 5.66 | 571.6 | 11.40 | 0.2 | 5 |
| Compound 41 | 0.03 | 2.4 | 0.85 | 567.1 | 9.10 | 1.0 | 5 |
| Compound 44 | 0.03 | 7.9 | 3.59 | 603.9 | 6.53 | −5.5 | 5 |
| Compound 43 | 0.03 | 8.6 | 2.04 | 619.2 | 14.61 | −8.1 | 5 |
| Compound 46 | 0.03 | 8.7 | 3.82 | 514.6 | 7.69 | 10.1 | 5 |
| Compound 40 | 0.03 | 27.5 | 12.31 | 417.0 | 21.05 | 27.2 | |

SEQUENCE TABLE

| | | SEQ ID |
|---|---|---|
| Description | Sequence | No. |
| G000282 | mU*mU*mA*CAGCCACGUCUACAGCAGUUUUAGAmG | 1 |
| sgRNA | mCmUmAmGmAmAmAmUmAmGmCAAGUUAAAAUAA | |
| targeting mouse | GGCUAGUCCGUUAUCAmAmCmUmUmGmAmAmAmA | |
| TTR | mAmGmUmGmGmCmAmCmCmGmAmGmUmCmGmGmU | |
| | mGmCmU*mU*mU*mU | |
| G000650 | mG*mA*mC*AAGCACCAGAAAGACCAGUUUUAGAmG | 2 |
| sgRNA | mCmUmAmGmAmAmAmUmAmGmCAAGUUAAAAUAA | |
| targeting | GGCUAGUCCGUUAUCAmAmCmUmUmGmAmAmAmA | |
| human B2M | mAmGmUmGmGmCmAmCmCmGmAmGmUmCmGmGmU | |
| | mGmCmU*mU*mU*mU | |
| mRNA | GGGUCCCGCAGUCGGCGUCCAGCGGCUCUGCUUGUU | 3 |
| encoding Cas9 | CGUGUGUGUGUCGUUGCAGGCCUUAUUCGGAUCCG | |
| | CCACCAUGGACAAGAAGUACAGCAUCGGACUGGAC | |
| | AUCGGAACAAACAGCGUCGGAUGGGCAGUCAUCAC | |
| | AGACGAAUACAAGGUCCCGAGCAAGAAGUUCAAGG | |
| | UCCUGGGAAACACAGACAGACACAGCAUCAAGAAG | |
| | AACCUGAUCGGAGCACUGCUGUUCGACAGCGGAGA | |
| | AACAGCAGAAGCAACAAGACUGAAGAGAACAGCAA | |
| | GAAGAAGAUACACAAGAAGAAAGAACAGAAUCUGC | |
| | UACCUGCAGGAAAUCUUCAGCAACGAAAUGGCAAA | |
| | GGUCGACGACAGCUUCUUCCACAGACUGGAAGAAA | |
| | GCUUCCUGGUCGAAGAAGACAAGAAGCACGAAAGA | |
| | CACCCGAUCUUCGGAAACAUCGUCGACGAAGUCGCA | |
| | UACCACGAAAAGUACCCGACAAUCUACCACCUGAGA | |
| | AAGAAGCUGGUCGACAGCACAGACAAGGCAGACCU | |
| | GAGACUGAUCUACCUGGCACUGGCACACAUGAUCA | |
| | AGUUCAGAGGACACUUCCUGAUCGAAGGAGACCUG | |
| | AACCCGGACAACAGCGACGUCGACAAGCUGUUCAUC | |
| | CAGCUGGUCCAGACAUACAACCAGCUGUUCGAAGA | |
| | AAACCCGAUCAACGCAAGCGGAGUCGACGCAAAGG | |
| | CAAUCCUGAGCGCAAGACUGAGCAAGAGCAGAAGA | |
| | CUGGAAAACCUGAUCGCACAGCUGCCGGGAGAAAA | |
| | GAAGAACGGACUGUUCGGAAACCUGAUCGCACUGA | |
| | GCCUGGGACUGACACCGAACUUCAAGAGCAACUUC | |
| | GACCUGGCAGAAGACGCAAAGCUGCAGCUGAGCAA | |
| | GGACACAUACGACGACGACCUGGACAACCUGCUGGC | |
| | ACAGAUCGGAGACCAGUACGCAGACCUGUUCCUGG | |
| | CAGCAAAGAACCUGAGCGACGCAAUCCUGCUGAGC | |
| | GACAUCCUGAGAGUCAACACAGAAAUCACAAAGGC | |
| | ACCGCUGAGCGCAAGCAUGAUCAAGAGAUACGACG | |
| | AACACCACCAGGACCUGACACUGCUGAAGGCACUGG | |
| | UCAGACAGCAGCUGCCGGAAAAGUACAAGGAAAUC | |
| | UUCUUCGACCAGAGCAAGAACGGAUACGCAGGAUA | |
| | CAUCGACGGAGGAGCAAGCCAGGAAGAAUUCUACA | |
| | AGUUCAUCAAGCCGAUCCUGGAAAAGAUGGACGGA | |
| | ACAGAAGAACUGCUGGUCAAGCUGAACAGAGAAGA | |
| | CCUGCUGAGAAAGCAGAGAACAUUCGACAACGGAA | |
| | GCAUCCCGCACCAGAUCCACCUGGGAGAACUGCACG | |
| | CAAUCCUGAGAAGACAGGAAGACUUCUACCCGUUC | |
| | CUGAAGGACAACAGAGAAAAGAUCGAAAAGAUCCU | |
| | GACAUUCAGAAUCCCGUACUACGUCGGACCGCUGGC | |
| | AAGAGGAAACAGCAGAUUCGCAUGGAUGACAAGAA | |
| | AGAGCGAAGAAACAAUCACACCGUGGAACUUCGAA | |
| | GAAGUCGUCGACAAGGGAGCAAGCGCACAGAGCUU | |
| | CAUCGAAAGAAUGACAAACUUCGACAAGAACCUGC | |
| | CGAACGAAAAGGUCCUGCCGAAGCACAGCCUGCUG | |
| | UACGAAUACUUCACAGUCUACAACGAACUGACAAA | |
| | GGUCAAGUACGUCACAGAAGGAAUGAGAAAGCCGG | |
| | CAUUCCUGAGCGGAGAACAGAAGAAGGCAAUCGUC | |
| | GACCUGCUGUUCAAGACAAACAGAAAGGUCACAGU | |
| | CAAGCAGCUGAAGGAAGACUACUUCAAGAAGAUCG | |
| | AAUGCUUCGACAGCGUCGAAAUCAGCGGAGUCGAA | |
| | GACAGAUUCAACGCAAGCCUGGGAACAUACCACGA | |
| | CCUGCUGAAGAUCAUCAAGGACAAGGACUUCCUGG | |
| | ACAACGAAGAAAACGAAGACAUCCUGGAAGACAUC | |
| | GUCCUGACACUGACACUGUUCGAAGACAGAGAAAU | |
| | GAUCGAAGAAAGACUGAAGACAUACGCACACCUGU | |
| | UCGACGACAAGGUCAUGAAGCAGCUGAAGAGAAGA | |
| | AGAUACACAGGAUGGGGAAGACUGAGCAGAAAGCU | |
| | GAUCAACGGAAUCAGAGACAAGCAGAGCGGAAAGA | |
| | CAAUCCUGGACUUCCUGAAGAGCGACGGAUUCGCA | |
| | AACAGAAACUUCAUGCAGCUGAUCCACGACGACAG | |
| | CCUGACAUUCAAGGAAGACAUCCAGAAGGCACAGG | |
| | UCAGCGGACAGGGAGACAGCCUGCACGAACACAUC | |
| | GCAAACCUGGCAGGAAGCCCGGCAAUCAAGAAGGG | |
| | AAUCCUGCAGACAGUCAAGGUCGUCGACGAACUGG | |
| | UCAAGGUCAUGGGAAGACACAAGCCGGAAAACAUC | |
| | GUCAUCGAAAUGGCAAGAGAAAACCAGACAACACA | |
| | GAAGGGACAGAAGAACAGCAGAGAAAGAAUGAAGA | |
| | GAAUCGAAGAAGGAAUCAAGGAACUGGGAAGCCAG | |
| | AUCCUGAAGGAACACCCGGUCGAAAACACACAGCU | |
| | GCAGAACGAAAAGCUGUACCUGUACUACCUGCAGA | |
| | ACGGAAGAGACAUGUACGUCGACCAGGAACUGGAC | |
| | AUCAACAGACUGAGCGACUACGACGUCGACCACAUC | |
| | GUCCCGCAGAGCUUCCUGAAGGACGACAGCAUCGAC | |
| | AACAAGGUCCUGACAAGAAGCGACAAGAACAGAGG | |
| | AAAGAGCGACAACGUCCCGAGCGAAGAAGUCGUCA | |
| | AGAAGAUGAAGAACUACUGGAGACAGCUGCUGAAC | |
| | GCAAAGCUGAUCACACAGAGAAAGUUCGACAACCU | |
| | GACAAAGGCAGAGAGAGGAGGACUGAGCGAACUGG | |
| | ACAAGGCAGGAUUCAUCAAGAGACAGCUGGUCGAA | |
| | ACAAGACAGAUCACAAAGCACGUCGCACAGAUCCU | |
| | GGACAGCAGAAUGAACACAAAGUACGACGAAAACG | |
| | ACAAGCUGAUCAGAGAAGUCAAGGUCAUCACACUG | |
| | AAGAGCAAGCUGGUCAGCGACUUCAGAAAGGACUU | |
| | CCAGUUCUACAAGGUCAGAGAAAUCAACAACUACC | |
| | ACCACGCACACGACGCAUACCUGAACGCAGUCGUCG | |
| | GAACAGCACUGAUCAAGAAGUACCCGAAGCUGGAA | |
| | AGCGAAUUCGUCUACGGAGACUACAAGGUCUACGA | |
| | CGUCAGAAAGAUGAUCGCAAAGAGCGAACAGGAAA | |
| | UCGGAAAGGCAACAGCAAAGUACUUCUUCUACAGC | |
| | AACAUCAUGAACUUCUUCAAGACAGAAAUCACACU | |
| | GGCAAACGGAGAAAUCAGAAAGAGACCGCUGAUCG | |
| | AAACAAACGGAGAAACAGGAGAAAUCGUCUGGGAC | |
| | AAGGGAAGAGACUUCGCAACAGUCAGAAAGGUCCU | |
| | GAGCAUGCCGCAGGUCAACAUCGUCAAGAAGACAG | |
| | AAGUCCAGACAGGAGGAUUCAGCAAGGAAAGCAUC | |
| | CUGCCGAAGAGAAACAGCGACAAGCUGAUCGCAAG | |
| | AAAGAAGGACUGGGACCCGAAGAAGUACGGAGGAU | |
| | UCGACAGCCCGACAGUCGCAUACAGCGUCCUGGUCG | |
| | UCGCAAAGGUCGAAAAGGGAAAGAGCAAGAAGCUG | |
| | AAGAGCGUCAAGGAACUGCUGGGAAUCACAAUCAU | |
| | GGAAAGAAGCAGCUUCGAAAAGAACCCGAUCGACU | |
| | UCCUGGAAGCAAAGGGAUACAAGGAAGUCAAGAAG | |
| | GACCUGAUCAUCAAGCUGCCGAAGUACAGCCUGUU | |
| | CGAACUGGAAAACGGAAGAAAGAGAAUGCUGGCAA | |
| | GCGCAGGAGAACUGCAGAAGGGAAACGAACUGGCA | |
| | CUGCCGAGCAAGUACGUCAACUUCCUGUACCUGGCA | |
| | AGCCACUACGAAAAGCUGAAGGGAAGCCCGGAAGA | |
| | CAACGAACAGAAGCAGCUGUUCGUCGAACAGCACA | |
| | AGCACUACCUGGACGAAAUCAUCGAACAGAUCAGC | |
| | GAAUUCAGCAAGAGAGUCAUCCUGGCAGACGCAAA | |
| | CCUGGACAAGGUCCUGAGCGCAUACAACAAGCACA | |
| | GAGACAAGCCGAUCAGAGAACAGGCAGAAAACAUC | |
| | AUCCACCUGUUCACACUGACAAACCUGGGAGCACCG | |
| | GCAGCAUUCAAGUACUUCGACACAACAAUCGACAG | |
| | AAAGAGAUACACAAGCACAAAGGAAGUCCUGGACG | |
| | CAACACUGAUCCACCAGAGCAUCACAGGACUGUACG | |
| | AAACAAGAAUCGACCUGAGCCAGCUGGGAGGAGAC | |
| | GGAGGAGGAAGCCCGAAGAAGAAGAGAAAGGUCUA | |
| | GCUAGCCAUCACAUUUAAAAGCAUCUCAGCCUACCA | |
| | UGAGAAUAAGAGAAAGAAAAUGAAGAUCAAUAGCU | |
| | UAUUCAUCUCUUUUUCUUUUUCGUUGGUGUAAAGC | |
| | CAACACCCUGUCUAAAAAACAUAAAUUUCUUUAAU | |
| | CAUUUUGCCUCUUUUCUCUGUGCUUCAAUUAAUAA | |
| | AAAAUGGAAAGAACCUCGAGAAAAAAAAAAAAAAA | |
| | AAAAAAAAAAAAAAAAAAAAAAAAAAAAAAAAAAA | |
| | AAAAAAAAAAAAAAAAAAAAAAAAAAAAAAAAAAA | |
| | AAAAAAAAAAAAAAAUCUAG | |
| G000502 | mA*mC*mA*CAAAUACCAGUCCAGCGGUUUUAGAmG | 4 |
| sgRNA | mCmUmAmGmAmAmAmUmAmGmCAAGUUAAAAUAA | |
| targeting mouse | GGCUAGUCCGUUAUCAmAmCmUmUmGmAmAmAmA | |
| TTR | mAmGmUmGmGmCmAmCmCmGmAmGmUmCmGmGmU | |
| | mGmCmU*mU*mU*mU | |

2′-O-methyl modifications and phosphorothioate linkages as represented below (m = 2′-OMe; * = phosphorothioate)

What is claimed is:

1. A compound of Formula (I) 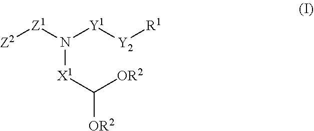
wherein, independently for each occurrence,
X¹ is C₅₋₁₁ alkylene,
Y¹ is C₃₋₁₁ alkylene,
Y² is 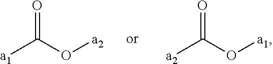
wherein a₁ is a bond to Y¹, and a₂ is a bond to R¹,
Z¹ is C₂₋₄ alkylene,
Z² is selected from —OH, —NH₂, —OC(═O)R³, —OC(═O)NHR³, —NHC(═O)NHR³, and —NHS(═O)₂R³,
R¹ is C₄₋₁₂ alkyl or C₃₋₁₂ alkenyl,
each R² is independently C₄₋₁₂ alkyl, and
R³ is C₁₋₃ alkyl,
or a salt thereof.

2. The compound of claim 1, wherein the salt is a pharmaceutically acceptable salt.

3. The compound of claim 1 or 2, wherein X¹ is linear C₅₋₁₁ alkylene.

4. The compound of any one of the preceding claims, wherein X¹ is linear C₆₋₁₀ alkylene.

5. The compound of any one of the preceding claims, wherein X¹ is linear C₆ alkylene, linear C₇ alkylene, linear C₈ alkylene, or linear C₉ alkylene.

6. The compound of any one of the preceding claims, wherein Y¹ is linear C₄₋₉ alkylene.

7. The compound of any one of the preceding claims, wherein Y¹ linear C₆₋₈ alkylene.

8. The compound of any one of the preceding claims, wherein Y¹ is linear C₇ alkylene.

9. The compound of any one of the preceding claims, wherein R¹ is C₄₋₁₂ alkenyl.

10. The compound of any one of the preceding claims, wherein R¹ is C₉ alkenyl.

11. The compound of any one of the preceding claims, wherein Y² is

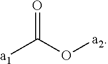

12. The compound of any one of the preceding claims, wherein Y¹, Y², and R¹ are selected to form a linear chain of 16-21 atoms.

13. The compound of any one of the preceding claims, wherein Y¹, Y², and R¹ are selected to form a linear chain of 16-18 atoms.

14. The compound of any one of the preceding claims, wherein Z¹ is linear C₂₋₄ alkylene.

15. The compound of any one of the preceding claims, wherein Z¹ is C₂ alkylene or C₃ alkylene.

16. The compound of any one of the preceding claims, wherein Z² is —OH.

17. The compound of any one of claims 1-15, wherein Z² is —NH₂.

18. The compound of any one of claims 1-15, wherein Z² is —OC(═O)R³, —OC(═O)NHR³, —NHC(═O)NHR³, or —NHS(═O)₂R³.

19. The compound of claim 18, wherein R³ is methyl.

20. The compound of any one of the preceding claims, wherein R¹ is linear C₄₋₁₂ alkyl.

21. The compound of any one of the preceding claims, wherein R¹ is linear C₈₋₁₀ alkyl.

22. The compound of any one of the preceding claims, wherein R¹ is linear C₉ alkyl.

23. The compound of any one of claims 1-19, wherein R¹ is branched C₆₋₁₂ alkyl.

24. The compound of claim 23, wherein R¹ is branched C₈ alkyl, branched C₉ alkyl, or branched C₁₀ alkyl.

25. The compound of any one of the preceding claims, wherein each R², independently, is linear C₅₋₁₂ alkyl.

26. The compound of any one of the preceding claims, wherein each R², independently, is linear C₆₋₈ alkyl.

27. The compound of any one of claims 1-24, wherein each R², independently, is branched C₅₋₁₂ alkyl.

28. The compound of claim 27, wherein each R², independently, is branched C₆₋₈ alkyl.

29. The compound of any one of the preceding claims, wherein X¹ and one of the R² moieties are selected to form a linear chain of 16-18 atoms, including the carbon and oxygen atoms of the acetal.

30. The compound of claim 1, wherein the compound is a compound of Formula (II) 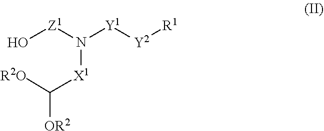
wherein, independently for each occurrence,
X¹ is C₅₋₁₁ alkylene,
Y¹ is C₃₋₁₀ alkylene,
Y² is 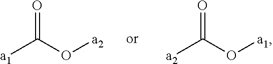
wherein a₁ is a bond to Y¹, and a₂ is a bond to R¹,
Z¹ is C₂₋₄ alkylene,
R¹ is C₄₋₁₂ alkyl or C₃₋₁₂ alkenyl,
each R² is independently C₄₋₁₂ alkyl,
or a salt thereof.

31. The compound of claim 30, wherein the salt is a pharmaceutically acceptable salt.

32. The compound of claim 30 or 31, wherein X¹ is linear C₅₋₁₁ alkylene.

33. The compound of claim 32, wherein X¹ is linear C₆₋₈ alkylene.

34. The compound of claim 33, wherein X¹ is linear C₇ alkylene.

35. The compound of any one of claims 30-34, wherein Y¹ is linear C₄₋₉ alkylene.

36. The compound of any one of claims 30-35, wherein Y¹ is linear C₅₋₉ alkylene.

37. The compound of any one of claims 30-36, wherein Y¹ is linear C₆₋₈ alkylene.

38. The compound of any one of claims 30-37, wherein Y¹ is linear C₇ alkylene.

39. The compound of any one of claims 30-38, wherein Y² is

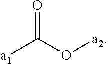

40. The compound of any one of claims 30-39, wherein R¹ is C₄₋₁₂ alkenyl.

41. The compound of any one of claims 30-40, wherein R¹ is C₉ alkenyl.

42. The compound of any one of claims 30-41, wherein Y¹, Y², and R¹ are selected to form a linear chain of 16-21 atoms.

43. The compound of any one of claims 30-42, wherein Y¹, Y², and R¹ are selected to form a linear chain of 16-18 atoms.

44. The compound of any one of claims 30-43, wherein Z¹ is linear C₂₋₄ alkylene.

45. The compound of any one of claims 30-44, wherein Z¹ is C₂ alkylene.

46. The compound of any one of claims 30-39 and 42-45, wherein R¹ is linear C₄₋₁₂ alkyl.

47. The compound of any one of claims 30-39 and 42-46, wherein R¹ is linear C₈₋₁₀ alkyl.

48. The compound of any one of claims 30-39 and 42-47, wherein R¹ is linear C₉ alkyl.

49. The compound of any one of claims 30-48, wherein each R² is C₅₋₁₂ alkyl.

50. The compound of any one of claims 30-49, wherein each R² is linear C₅₋₁₂ alkyl.

51. The compound of any one of claims 30-50, wherein each R² is linear C₆₋₁₀ alkyl.

52. The compound of any one of claims 30-51, wherein each R² is linear C₆₋₈ alkyl.

53. The compound of any one of claims 30-52, wherein X¹ and one of the R² moieties are selected to form a linear chain of 16-18 atoms, including the carbon and oxygen atoms of the acetal.

54. The compound of claim 1, wherein the compound is selected from: 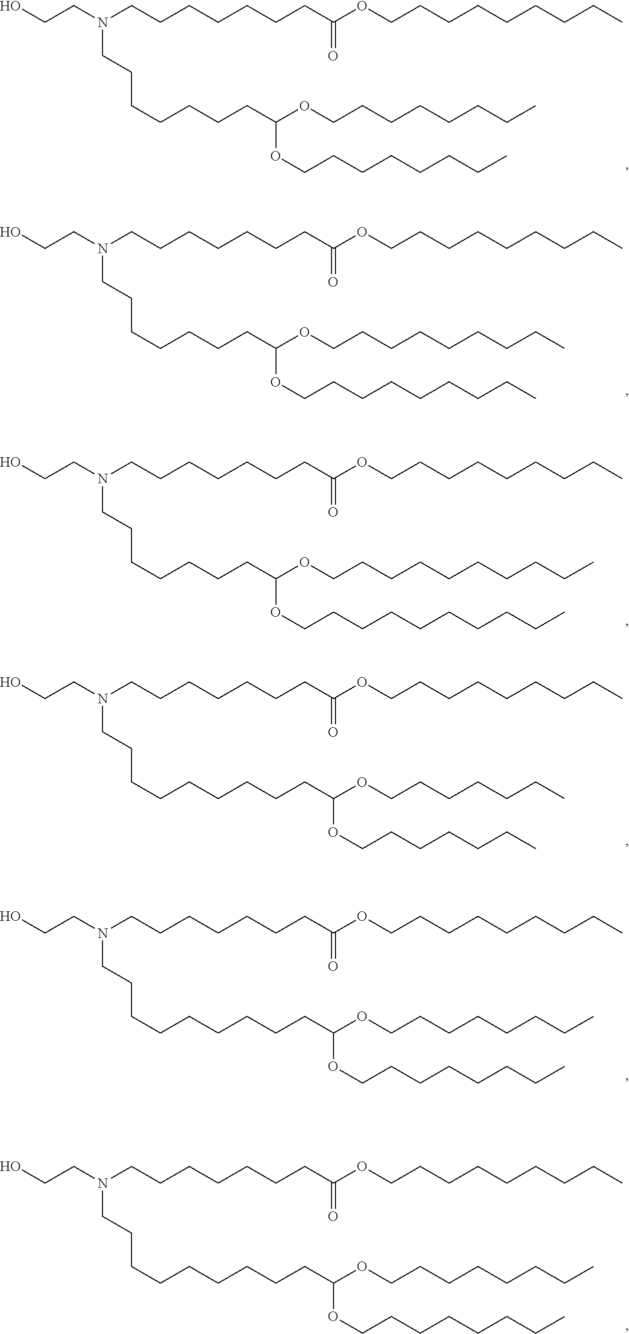 [SMILES: CCCCCCCCCOC(=O)CCCCCCCN(CCO)CCCCCCCC(OCCCCCCCC)OCCCCCCCC ; CCCCCCCCCOC(=O)CCCCCCCN(CCO)CCCCCCCC(OCCCCCCCCC)OCCCCCCCCC ; CCCCCCCCCCOC(CCCCCCCN(CCO)CCCCCCCC(=O)OCCCCCCCCC)OCCCCCCCCCC ; CCCCCCCCCOC(=O)CCCCCCCN(CCO)CCCCCCCCCC(OCCCCCCC)OCCCCCCC ; CCCCCCCCCOC(=O)CCCCCCCN(CCO)CCCCCCCCCC(OCCCCCCCC)OCCCCCCCC ; CCCCCCCCCOC(=O)CCCCCCCN(CCO)CCCCCCCCCC(OCCCCCCCC)OCCCCCCCC] 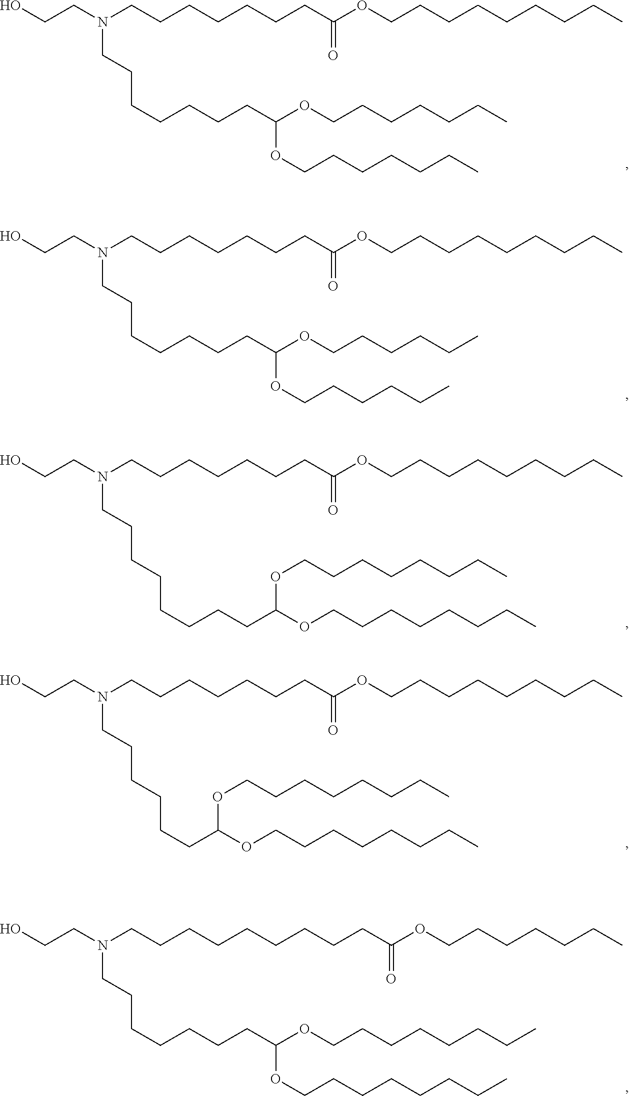 [SMILES: CCCCCCCCCOC(=O)CCCCCCCN(CCO)CCCCCCCC(OCCCCCCC)OCCCCCCC ; CCCCCCCCCOC(=O)CCCCCCCN(CCO)CCCCCCCC(OCCCCCC)OCCCCCC ; CCCCCCCCCOC(=O)CCCCCCCN(CCO)CCCCCCCCC(OCCCCCCCC)OCCCCCCCC ; CCCCCCCCCOC(=O)CCCCCCCN(CCO)CCCCCCC(OCCCCCCCC)OCCCCCCCC ; CCCCCCCCOC(CCCCCCCN(CCO)CCCCCCCCCC(=O)OCCCCCCC)OCCCCCCCC] 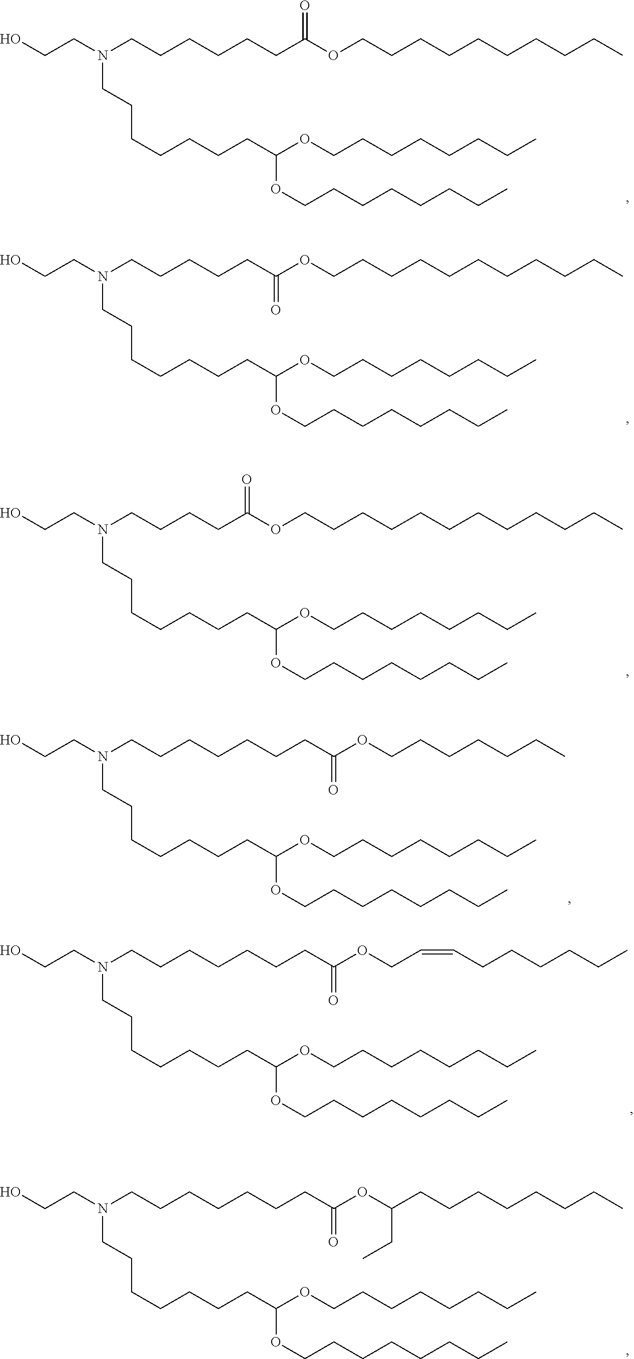 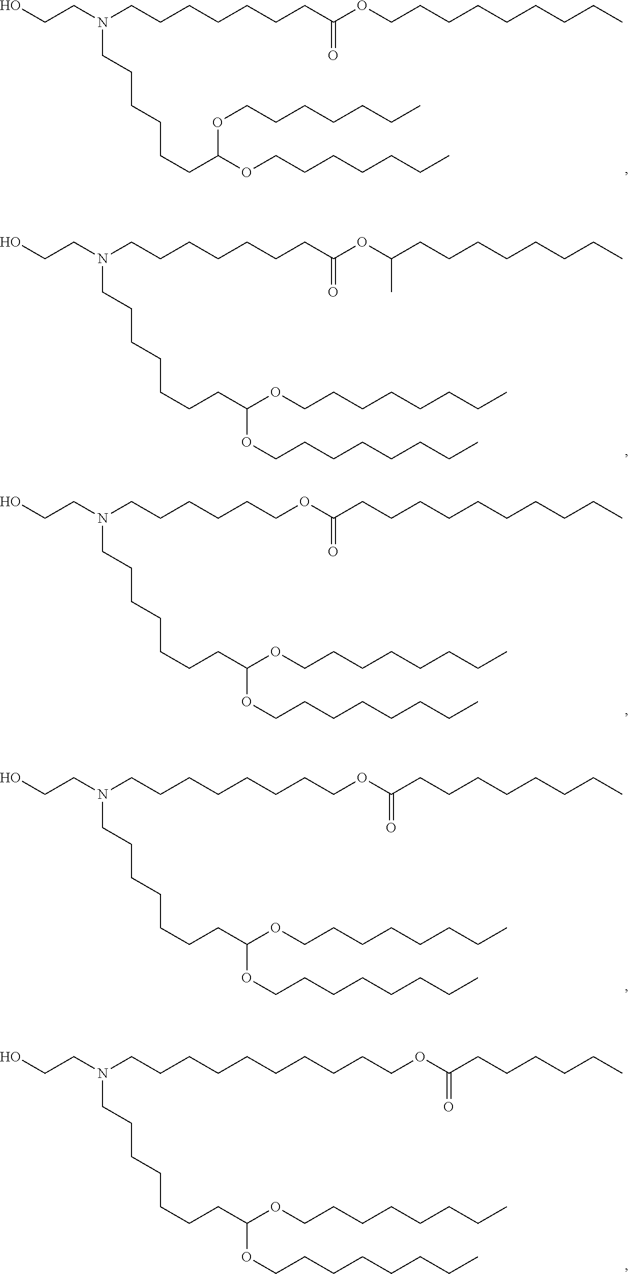 [SMILES: CCCCCCCCCOC(=O)CCCCCCCN(CCO)CCCCCCC(OCCCCCCC)OCCCCCCC ; CCCCCCCCOC(CCCCCCCN(CCO)CCCCCCCC(=O)OC(C)CCCCCCCC)OCCCCCCCC ; CCCCCCCCCCC(=O)OCCCCCCN(CCO)CCCCCCCC(OCCCCCCCC)OCCCCCCCC ; CCCCCCCCOC(CCCCCCCN(CCO)CCCCCCCCOC(=O)CCCCCCCC)OCCCCCCCC ; CCCCCCCCOC(CCCCCCCN(CCO)CCCCCCCCCCOC(=O)CCCCCC)OCCCCCCCC] 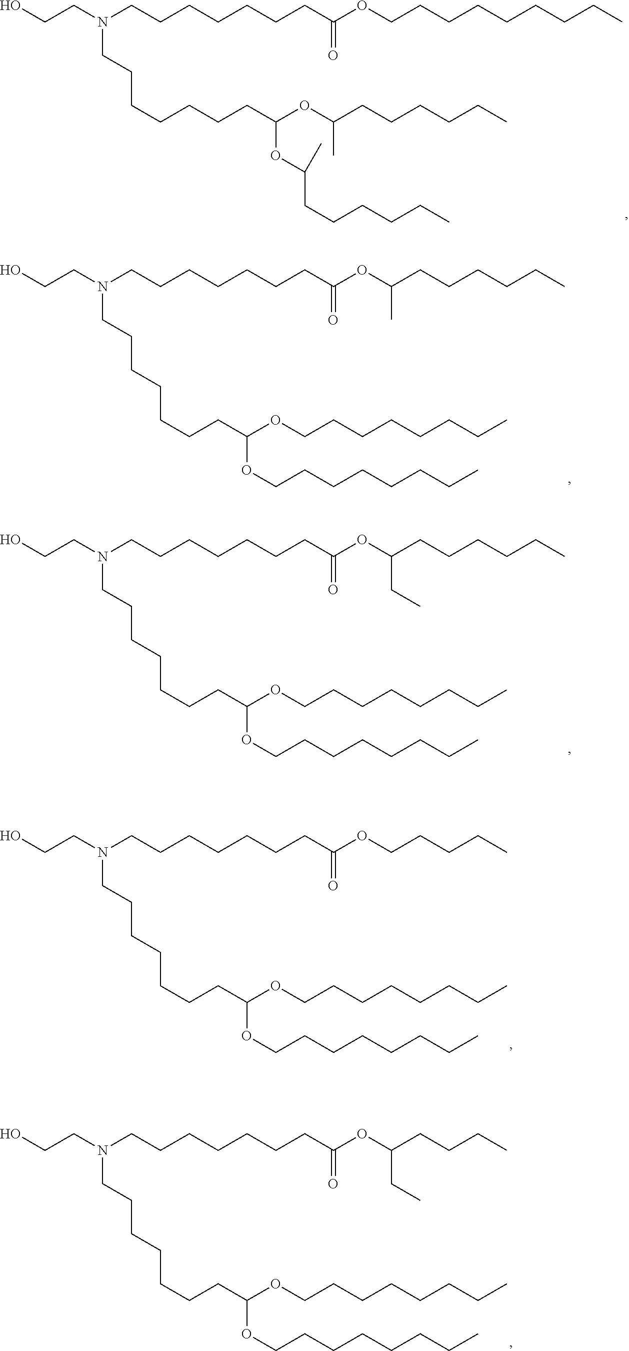 [SMILES: CCCCCCCCCOC(=O)CCCCCCCN(CCO)CCCCCCCC(OC(C)CCCCCC)OC(C)CCCCCC ; CCCCCCCCOC(CCCCCCCN(CCO)CCCCCCCC(=O)OC(C)CCCCCC)OCCCCCCCC ; CCCCCCCCOC(CCCCCCCN(CCO)CCCCCCCC(=O)OC(CC)CCCCCC)OCCCCCCCC ; CCCCCCCCOC(CCCCCCCN(CCO)CCCCCCCC(=O)OCCCCC)OCCCCCCCC ; CCCCCCCCOC(CCCCCCCN(CCO)CCCCCCCC(=O)OC(CC)CCCC)OCCCCCCCC] 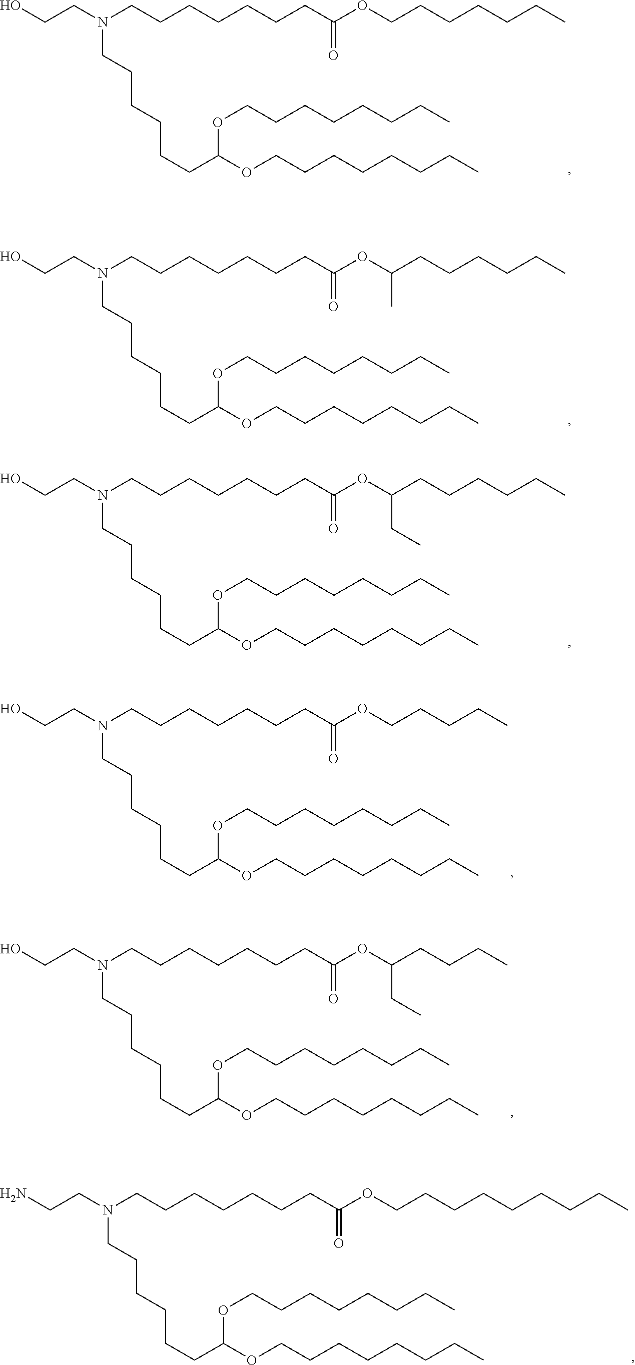 [SMILES: CCCCCCCCOC(CCCCCCN(CCO)CCCCCCCC(=O)OCCCCCCC)OCCCCCCCC ; CCCCCCCCOC(CCCCCCN(CCO)CCCCCCCC(=O)OC(C)CCCCCC)OCCCCCCCC ; CCCCCCCCOC(CCCCCCN(CCO)CCCCCCCC(=O)OC(CC)CCCCCC)OCCCCCCCC ; CCCCCCCCOC(CCCCCCN(CCO)CCCCCCCC(=O)OCCCCC)OCCCCCCCC ; CCCCCCCCOC(CCCCCCN(CCO)CCCCCCCC(=O)OC(CC)CCCC)OCCCCCCCC ; CCCCCCCCCOC(=O)CCCCCCCN(CCN)CCCCCCC(OCCCCCCCC)OCCCCCCCC] 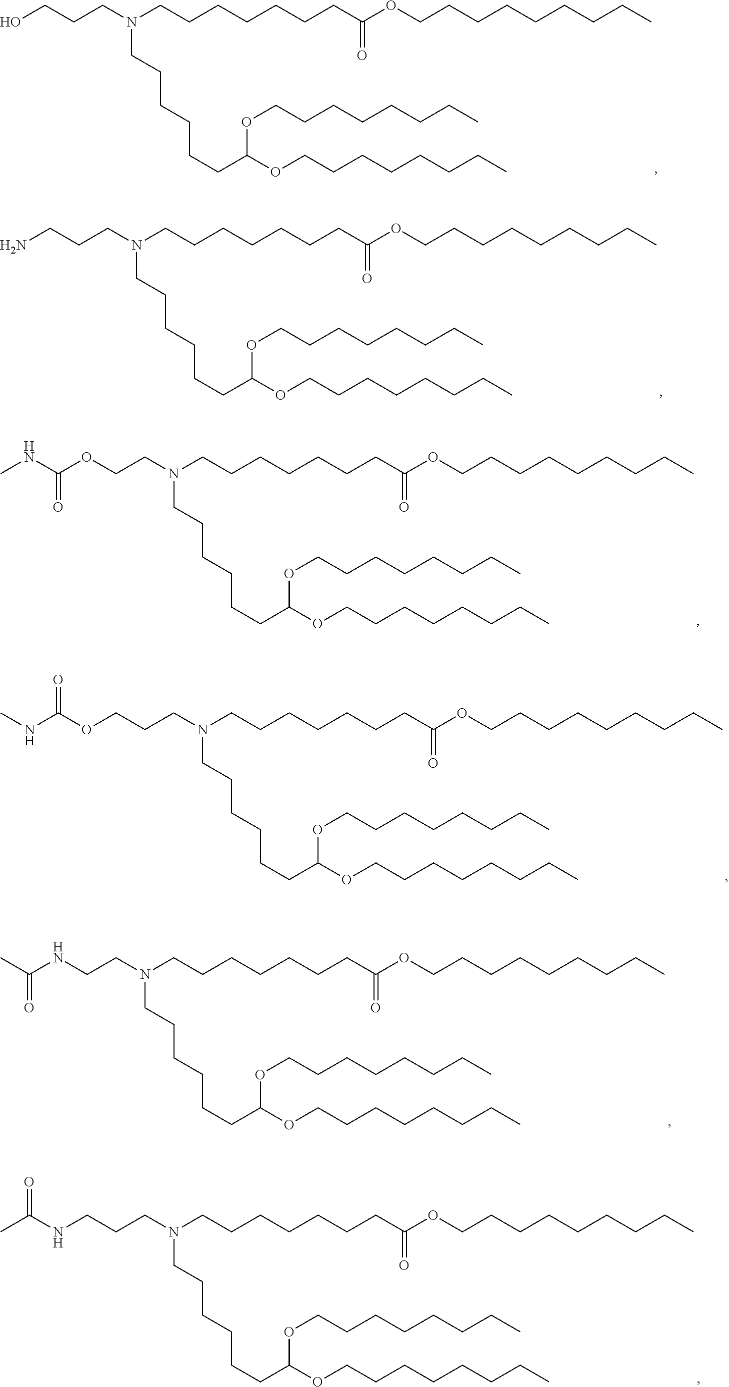 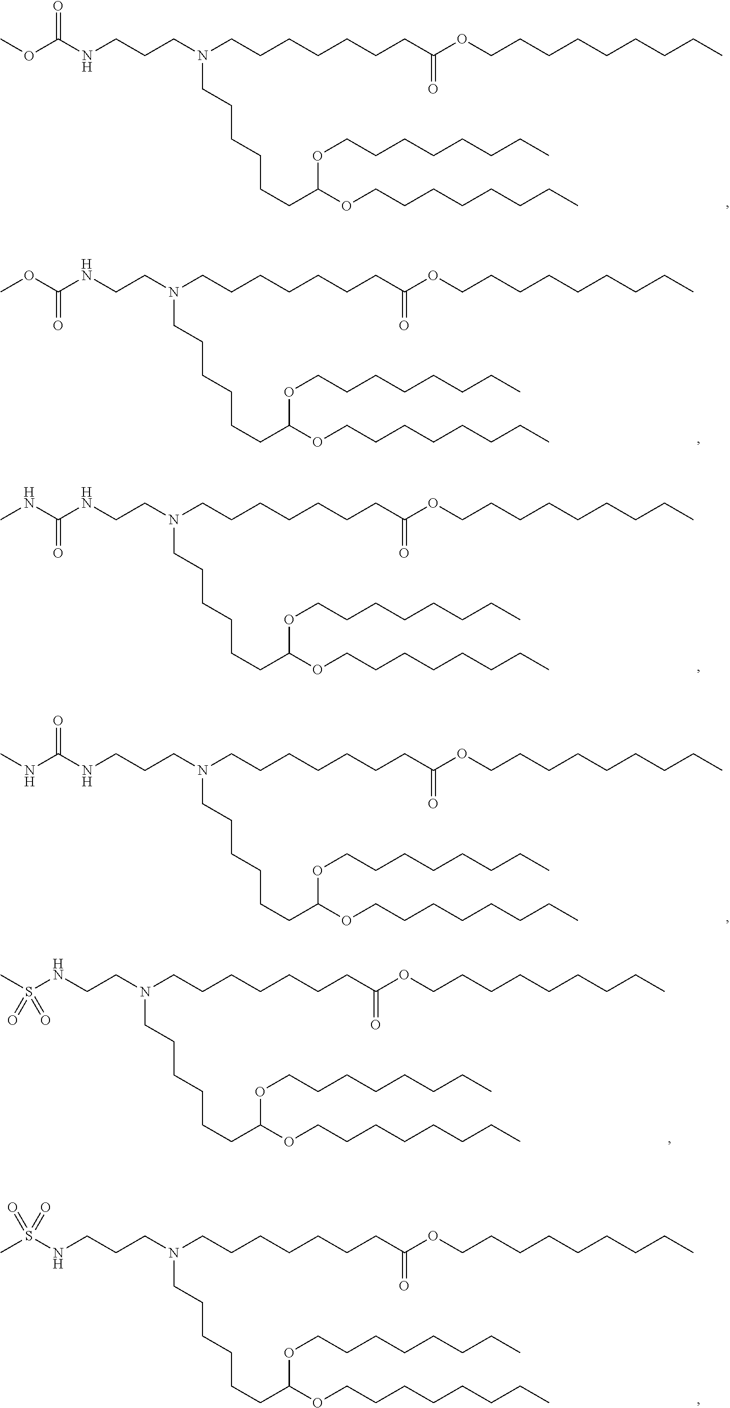 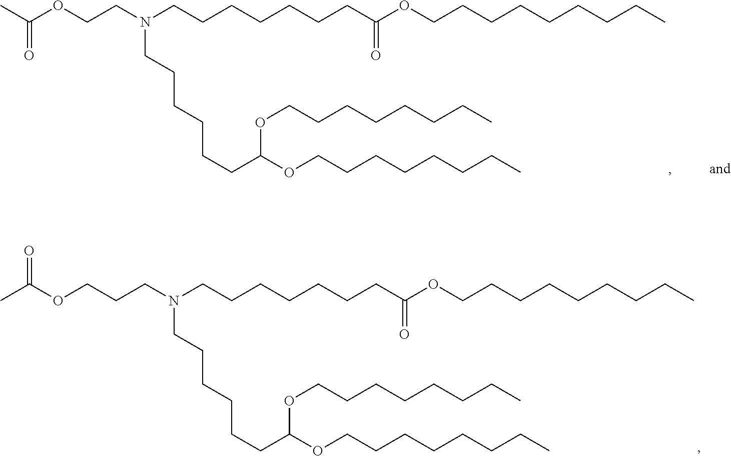
or a salt thereof.

55. The compound of claim 55, wherein the salt is a pharmaceutically acceptable salt.

56. The compound of any one of the preceding claims, wherein the pKa of the protonated form of the compound is from about 5.1 to about 8.0.

57. The compound of any one of the preceding claims, wherein the pKa of the protonated form of the compound is from about 5.7 to about 6.4.

58. The compound of any one of the preceding claims, wherein the pKa of the protonated form of the compound is from about 5.8 to about 6.2.

59. The compound of any one of claims 1-56, wherein the pKa of the protonated form of the compound is from about 5.5 to about 6.0.

60. The compound of claim 59, wherein the pKa of the protonated form of the compound is from about 6.1 to about 6.3.

61. A composition comprising a compound of any one of the preceding claims and a lipid component.

62. The composition of claim 61, wherein the composition comprises about 50% of the compound of any one of the preceding claims and a lipid component.

63. The composition of claim 61 or 62, wherein the composition is a LNP composition.

64. The composition of any one of claims 61-63, wherein the lipid component comprises a helper lipid and a PEG lipid.

65. The composition of any one of claims 61-64, wherein the lipid component comprises a helper lipid, a PEG lipid, and a neutral lipid.

66. The composition of any one of claims 61-65, further comprising a cryoprotectant.

67. The composition of any one of claims 61-66, further comprising a buffer.

68. The composition of any one of claims 61-67, further comprising a nucleic acid component.

69. The composition of claim 68, wherein the nucleic acid component is an RNA or DNA component.

70. The composition of claim 68 or 69, wherein the composition has an N/P ratio of about 3-10.

71. The composition of claim 70, wherein the N/P ratio is about 6±1.

72. The composition of claim 70, wherein the N/P ratio is about 6±0.5.

73. The composition of claim 70, wherein the N/P ratio is about 6.

74. The composition of any one of claims 61-73, comprising a RNA component, wherein the RNA component comprises a mRNA.

75. The composition of claim 74, wherein the RNA component comprises a RNA-guided DNA-binding agent, such as a Cas nuclease mRNA.

76. The composition of claim 74 or 75, wherein the RNA component comprises a Class 2 Cas nuclease mRNA.

77. The composition of any one of claims 74-76, wherein the RNA component comprises a Cas9 nuclease mRNA.

78. The composition of any one of claims 74-77, wherein the mRNA is a modified mRNA.

79. The composition of any one of claims 74-78, wherein the RNA component comprises a gRNA nucleic acid.

80. The composition of claim 79, wherein the gRNA nucleic acid is a gRNA.

81. The composition of any one of claims 74-78, wherein the RNA component comprises a Class 2 Cas nuclease mRNA and a gRNA.

82. The composition of any one of claims 79-81, wherein the gRNA nucleic acid is or encodes a dual-guide RNA (dgRNA).

83. The composition of any one of claims 79-81, wherein the gRNA nucleic acid is or encodes a single-guide RNA (sgRNA).

84. The composition of any one of claims 79-83, wherein the gRNA is a modified gRNA.

85. The composition of claim 84, wherein the modified gRNA comprises a modification at one or more of the first five nucleotides at a 5′ end.

86. The composition of claims 84 or 85, wherein the modified gRNA comprises a modification at one or more of the last five nucleotides at a 3′ end.

87. The composition of any one of claims 61-86, further comprising at least one template nucleic acid.

88. A method of gene editing, comprising contacting a cell with a composition of any one of claims 61-87.

89. A method of cleaving a DNA, comprising contacting a cell with a composition of any one of claims 61-87.

90. The method of claim 89, wherein the contacting step results in a single stranded DNA nick.

91. The method of claim 89, wherein the contacting step results in a double-stranded DNA break.

92. The method of claim 88, wherein the composition comprises a Class 2 Cas mRNA and a guide RNA nucleic acid.

93. The method of claims 88 or 92, further comprising introducing at least one template nucleic acid into the cell.

94. The method of claim 93, comprising contacting the cell with a composition comprising a template nucleic acid.

95. The method of any one of claims 88-94, wherein the method comprises administering the composition to an animal.

96. The method of any one of claims 88-95, wherein the method comprises administering the composition to a human.

97. The method of any one of claims 88-94, wherein the method comprises administering the composition to a cell.

98. The method of claim 97, wherein the cell is a eukaryotic cell.

99. The method of claim 88, wherein the method comprises administering the mRNA formulated in a first LNP composition and a second LNP composition comprising one or more of an mRNA, a gRNA, a gRNA nucleic acid, and a template nucleic acid.

100. The method of claim 99, wherein the first and second LNP compositions are administered simultaneously.

101. The method of claim 99, wherein the first and second LNP compositions are administered sequentially.

102. The method of claim 99, wherein the method comprises administering the mRNA and the guide RNA nucleic acid formulated in a single LNP composition.

103. The method of any one of claims 88-102, wherein the gene editing results in a gene knockout.

104. The method of any one of claims 88-102, wherein the gene editing results in a gene correction.


SEQUENCE LISTING (4 sequences)

SEQ ID NO: 1 - RNA, 100 nt, Artificial Sequence
  source: /note=“Description of Artificial Sequence Synthetic polynucleotide”
uuacagccacgucuacagcaguuuuagagcuagaaauagcaaguuaaaauaaggcuaguccguuaucaacuugaaaaaguggcaccgagucggugcuuuu

SEQ ID NO: 2 - RNA, 100 nt, Artificial Sequence
  source: /note=“Description of Artificial Sequence Synthetic polynucleotide”
gacaagcaccagaaagaccaguuuuagagcuagaaauagcaaguuaaaauaaggcuaguccguuaucaacuugaaaaaguggcaccgagucggugcuuuu

SEQ ID NO: 3 - RNA, 4516 nt, Artificial Sequence
  source: /note=“Description of Artificial Sequence Synthetic polynucleotide”
gggucccgcagucggcguccagcggcucugcuuguucgugugugugucguugcaggccuuauucggauccgccaccauggacaagaaguacagcaucggacuggacaucggaacaaacagcgucggaugggcagucaucacagacgaauacaaggucccgagcaagaaguucaagguccugggaaacacagacagacacagcaucaagaagaaccugaucggagcacugcuguucgacagcggagaaacagcagaagcaacaagacugaagagaacagcaagaagaagauacacaagaagaaagaacagaaucugcuaccugcaggaaaucuucagcaacgaaauggcaaaggucgacgacagcuucuuccacagacuggaagaaagcuuccuggucgaagaagacaagaagcacgaaagacacccgaucuucggaaacaucgucgacgaagucgcauaccacgaaaaguacccgacaaucuaccaccugagaaagaagcuggucgacagcacagacaaggcagaccugagacugaucuaccuggcacuggcacacaugaucaaguucagaggacacuuccugaucgaaggagaccugaacccggacaacagcgacgucgacaagcuguucauccagcugguccagacauacaaccagcuguucgaagaaaacccgaucaacgcaagcggagucgacgcaaaggcaauccugagcgcaagacugagcaagagcagaagacuggaaaaccugaucgcacagcugccgggagaaaagaagaacggacuguucggaaaccugaucgcacugagccugggacugacaccgaacuucaagagcaacuucgaccuggcagaagacgcaaagcugcagcugagcaaggacacauacgacgacgaccuggacaaccugcuggcacagaucggagaccaguacgcagaccuguuccuggcagcaaagaaccugagcgacgcaauccugcugagcgacauccugagagucaacacagaaaucacaaaggcaccgcugagcgcaagcaugaucaagagauacgacgaacaccaccaggaccugacacugcugaaggcacuggucagacagcagcugccggaaaaguacaaggaaaucuucuucgaccagagcaagaacggauacgcaggauacaucgacggaggagcaagccaggaagaauucuacaaguucaucaagccgauccuggaaaagauggacggaacagaagaacugcuggucaagcugaacagagaagaccugcugagaaagcagagaacauucgacaacggaagcaucccgcaccagauccaccugggagaacugcacgcaauccugagaagacaggaagacuucuacccguuccugaaggacaacagagaaaagaucgaaaagauccugacauucagaaucccguacuacgucggaccgcuggcaagaggaaacagcagauucgcauggaugacaagaaagagcgaagaaacaaucacaccguggaacuucgaagaagucgucgacaagggagcaagcgcacagagcuucaucgaaagaaugacaaacuucgacaagaaccugccgaacgaaaagguccugccgaagcacagccugcuguacgaauacuucacagucuacaacgaacugacaaaggucaaguacgucacagaaggaaugagaaagccggcauuccugagcggagaacagaagaaggcaaucgucgaccugcuguucaagacaaacagaaaggucacagucaagcagcugaaggaagacuacuucaagaagaucgaaugcuucgacagcgucgaaaucagcggagucgaagacagauucaacgcaagccugggaacauaccacgaccugcugaagaucaucaaggacaaggacuuccuggacaacgaagaaaacgaagacauccuggaagacaucguccugacacugacacuguucgaagacagagaaaugaucgaagaaagacugaagacauacgcacaccuguucgacgacaaggucaugaagcagcugaagagaagaagauacacaggauggggaagacugagcagaaagcugaucaacggaaucagagacaagcagagcggaaagacaauccuggacuuccugaagagcgacggauucgcaaacagaaacuucaugcagcugauccacgacgacagccugacauucaaggaagacauccagaaggcacaggucagcggacagggagacagccugcacgaacacaucgcaaaccuggcaggaagcccggcaaucaagaagggaauccugcagacagucaaggucgucgacgaacuggucaaggucaugggaagacacaagccggaaaacaucgucaucgaaauggcaagagaaaaccagacaacacagaagggacagaagaacagcagagaaagaaugaagagaaucgaagaaggaaucaaggaacugggaagccagauccugaaggaacacccggucgaaaacacacagcugcagaacgaaaagcuguaccuguacuaccugcagaacggaagagacauguacgucgaccaggaacuggacaucaacagacugagcgacuacgacgucgaccacaucgucccgcagagcuuccugaaggacgacagcaucgacaacaagguccugacaagaagcgacaagaacagaggaaagagcgacaacgucccgagcgaagaagucgucaagaagaugaagaacuacuggagacagcugcugaacgcaaagcugaucacacagagaaaguucgacaaccugacaaaggcagagagaggaggacugagcgaacuggacaaggcaggauucaucaagagacagcuggucgaaacaagacagaucacaaagcacgucgcacagauccuggacagcagaaugaacacaaaguacgacgaaaacgacaagcugaucagagaagucaaggucaucacacugaagagcaagcuggucagcgacuucagaaaggacuuccaguucuacaaggucagagaaaucaacaacuaccaccacgcacacgacgcauaccugaacgcagucgucggaacagcacugaucaagaaguacccgaagcuggaaagcgaauucgucuacggagacuacaaggucuacgacgucagaaagaugaucgcaaagagcgaacaggaaaucggaaaggcaacagcaaaguacuucuucuacagcaacaucaugaacuucuucaagacagaaaucacacuggcaaacggagaaaucagaaagagaccgcugaucgaaacaaacggagaaacaggagaaaucgucugggacaagggaagagacuucgcaacagucagaaagguccugagcaugccgcaggucaacaucgucaagaagacagaaguccagacaggaggauucagcaaggaaagcauccugccgaagagaaacagcgacaagcugaucgcaagaaagaaggacugggacccgaagaaguacggaggauucgacagcccgacagucgcauacagcguccuggucgucgcaaaggucgaaaagggaaagagcaagaagcugaagagcgucaaggaacugcugggaaucacaaucauggaaagaagcagcuucgaaaagaacccgaucgacuuccuggaagcaaagggauacaaggaagucaagaaggaccugaucaucaagcugccgaaguacagccuguucgaacuggaaaacggaagaaagagaaugcuggcaagcgcaggagaacugcagaagggaaacgaacuggcacugccgagcaaguacgucaacuuccuguaccuggcaagccacuacgaaaagcugaagggaagcccggaagacaacgaacagaagcagcuguucgucgaacagcacaagcacuaccuggacgaaaucaucgaacagaucagcgaauucagcaagagagucauccuggcagacgcaaaccuggacaagguccugagcgcauacaacaagcacagagacaagccgaucagagaacaggcagaaaacaucauccaccuguucacacugacaaaccugggagcaccggcagcauucaaguacuucgacacaacaaucgacagaaagagauacacaagcacaaaggaaguccuggacgcaacacugauccaccagagcaucacaggacuguacgaaacaagaaucgaccugagccagcugggaggagacggaggaggaagcccgaagaagaagagaaaggucuagcuagccaucacauuuaaaagcaucucagccuaccaugagaauaagagaaagaaaaugaagaucaauagcuuauucaucucuuuuucuuuuucguugguguaaagccaacacccugucuaaaaaacauaaauuucuuuaaucauuuugccucuuuucucugugcuucaauuaauaaaaaauggaaagaaccucgagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaucuag

SEQ ID NO: 4 - RNA, 100 nt, Artificial Sequence
  source: /note=“Description of Artificial Sequence Synthetic polynucleotide”
acacaaauaccaguccagcgguuuuagagcuagaaauagcaaguuaaaauaaggcuaguccguuaucaacuugaaaaaguggcaccgagucggugcuuuu